SEQUENCE LISTING (190 sequences)

SEQ ID NO: 1 - protein, 70 aa, Homo sapiens
ASLAADTPTACCFSYTSRQIPQNFIADYFETSSQCSKPGVIFLTKRSRQVCADPSEEWVQKYVSDLELSA

SEQ ID NO: 2 - protein, 70 aa, Homo sapiens
APLAADTPTACCFSYTSRQIPQNFIADYFETSSQCSKPSVIFLTKRGRQVCADPSEEWVQKYVSDLELSA

SEQ ID NO: 3 - protein, 70 aa, synthetic construct
ASLAADTPTACCFSYTSRQIPQNFIAAYFETSSQCSKPGVIFLTKRSRQVCADPSEEWVQKYVSDLELSA

SEQ ID NO: 4 - protein, 70 aa, synthetic construct
ASLAADTPTACCFSYTSRQIPQNFIADYFETSSQCSKPGVIFLTKRSRQVCADPSEEWVQKYVSDLALSA

SEQ ID NO: 5 - protein, 70 aa, synthetic construct
APLAADTPTACCFSYTSRQIPQNFIAAYFETSSQCSKPSVIFLTKRGRQVCADPSEEWVQKYVSDLELSA

SEQ ID NO: 6 - protein, 70 aa, synthetic construct
APLAADTPTACCFSYTSRQIPQNFIADYFETSSQCSKPSVIFLTKRGRQVCADPSEEWVQKYVSDLALSA

SEQ ID NO: 7 - protein, 70 aa, synthetic construct
ASLAADTPTACCFSYTSRQIPQNFIAAYFETSSQCSKPGVIFLTKRSRQVCADPSEEWVQKYVSDLALSA

SEQ ID NO: 8 - protein, 70 aa, synthetic construct
APLAADTPTACCFSYTSRQIPQNFIAAYFETSSQCSKPSVIFLTKRGRQVCADPSEEWVQKYVSDLALSA

SEQ ID NO: 9 - DNA, 180 bp, unidentified
  source 1..180: mol_type = genomic DNA
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 10 - protein, 27 aa, unidentified
GYIPEAPRDGQAYVRKDGEWVLLSTFL

SEQ ID NO: 11 - DNA, 117 bp, unidentified
  source 1..117: mol_type = genomic DNA
aagcctctggacggagagtatttcactctccagatccggggccccgaaaggttcgaaatgttccgggagcttaacgaggccttggagctgaaagacgcacaggccggaaaggaaccg

SEQ ID NO: 12 - protein, 10 aa, synthetic construct
GGGSSGGGSG

SEQ ID NO: 13 - DNA, 333 bp, Homo sapiens
  source 1..333: mol_type = genomic DNA
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 14 - protein, 27 aa, unidentified
ELKTPLGDTTHTEPKSCDTPPPCPRCP

SEQ ID NO: 15 - DNA, 81 bp, unidentified
  source 1..81: mol_type = genomic DNA
gagctcaaaaccccacttggtgacacaactcacacagagcccaaatcttgtgacacacctcccccgtgcccaaggtgccca

SEQ ID NO: 16 - protein, 107 aa, unidentified
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - DNA, 321 bp, unidentified
  source 1..321: mol_type = genomic DNA
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 18 - protein, 144 aa, synthetic construct
ELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 19 - DNA, 432 bp, synthetic construct
  source 1..432: mol_type = other DNA
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 20 - protein, 9 aa, unidentified
CTELKLSDY

SEQ ID NO: 21 - protein, 9 aa, unidentified
NLVPMVATV

SEQ ID NO: 22 - protein, 9 aa, unidentified
KLVANNTRL

SEQ ID NO: 23 - protein, 5 aa, synthetic construct
GGGGS

SEQ ID NO: 24 - protein, 5 aa, synthetic construct
GGGSS

SEQ ID NO: 25 - protein, 5 aa, synthetic construct
GGGSG

SEQ ID NO: 26 - protein, 5 aa, synthetic construct
GGSGG

SEQ ID NO: 27 - protein, 6 aa, synthetic construct
SGSSGS

SEQ ID NO: 28 - protein, 10 aa, synthetic construct
GGGGSGGGGS

SEQ ID NO: 29 - protein, 5 aa, synthetic construct
  REPEAT 1..5: note = m
GGGGS

SEQ ID NO: 30 - protein, 5 aa, synthetic construct
  REPEAT 1..5: note = m
GGGSS

SEQ ID NO: 31 - protein, 5 aa, synthetic construct
  REPEAT 1..5: note = m
GGSGG

SEQ ID NO: 32 - protein, 5 aa, synthetic construct
  REPEAT 1..5: note = m
GGGSG

SEQ ID NO: 33 - protein, 6 aa, synthetic construct
  REPEAT 1..6: note = m
SGSSGS

SEQ ID NO: 34 - protein, 5 aa, synthetic construct
LGGGS

SEQ ID NO: 35 - protein, 5 aa, synthetic construct
GLGGS

SEQ ID NO: 36 - protein, 5 aa, synthetic construct
GGLGS

SEQ ID NO: 37 - protein, 5 aa, synthetic construct
GGGLS

SEQ ID NO: 38 - protein, 5 aa, synthetic construct
GGGGL

SEQ ID NO: 39 - protein, 5 aa, synthetic construct
LGGSG

SEQ ID NO: 40 - protein, 5 aa, synthetic construct
GLGSG

SEQ ID NO: 41 - protein, 5 aa, synthetic construct
GGLSG

SEQ ID NO: 42 - protein, 5 aa, synthetic construct
GGGLG

SEQ ID NO: 43 - protein, 5 aa, synthetic construct
GGGSL

SEQ ID NO: 44 - protein, 5 aa, synthetic construct
LGGSS

SEQ ID NO: 45 - protein, 5 aa, synthetic construct
GLGSS

SEQ ID NO: 46 - protein, 5 aa, synthetic construct
GGLSS

SEQ ID NO: 47 - protein, 5 aa, synthetic construct
LGLGS

SEQ ID NO: 48 - protein, 5 aa, synthetic construct
GLGLS

SEQ ID NO: 49 - protein, 5 aa, synthetic construct
GLLGS

SEQ ID NO: 50 - protein, 5 aa, synthetic construct
LGGLS

SEQ ID NO: 51 - protein, 5 aa, synthetic construct
GLGGL

SEQ ID NO: 52 - protein, 5 aa, synthetic construct
LGLSG

SEQ ID NO: 53 - protein, 5 aa, synthetic construct
GLLSG

SEQ ID NO: 54 - protein, 5 aa, synthetic construct
GGLSL

SEQ ID NO: 55 - protein, 5 aa, synthetic construct
GGLLG

SEQ ID NO: 56 - protein, 5 aa, synthetic construct
GLGSL

SEQ ID NO: 57 - protein, 5 aa, synthetic construct
LGLSS

SEQ ID NO: 58 - protein, 5 aa, synthetic construct
GGLLS

SEQ ID NO: 59 - protein, 10 aa, synthetic construct
LGGGSGGGGS

SEQ ID NO: 60 - protein, 10 aa, synthetic construct
GLGGSGGGGS

SEQ ID NO: 61 - protein, 10 aa, synthetic construct
GGLGSGGGGS

SEQ ID NO: 62 - protein, 10 aa, synthetic construct
GGGLSGGGGS

SEQ ID NO: 63 - protein, 10 aa, synthetic construct
GGGGLGGGGS

SEQ ID NO: 64 - protein, 10 aa, synthetic construct
LGGSGGGGSG

SEQ ID NO: 65 - protein, 10 aa, synthetic construct
GLGSGGGGSG

SEQ ID NO: 66 - protein, 10 aa, synthetic construct
GGLSGGGGSG

SEQ ID NO: 67 - protein, 10 aa, synthetic construct
GGGLGGGGSG

SEQ ID NO: 68 - protein, 10 aa, synthetic construct
GGGSLGGGSG

SEQ ID NO: 69 - protein, 10 aa, synthetic construct
LGGSSGGGSS

SEQ ID NO: 70 - protein, 10 aa, synthetic construct
GLGSSGGGSS

SEQ ID NO: 71 - protein, 10 aa, synthetic construct
GGLSSGGGSS

SEQ ID NO: 72 - protein, 10 aa, synthetic construct
GGGLSGGGSS

SEQ ID NO: 73 - protein, 10 aa, synthetic construct
GGGSLGGGSS

SEQ ID NO: 74 - protein, 10 aa, synthetic construct
LGGGSLGGGS

SEQ ID NO: 75 - protein, 10 aa, synthetic construct
GLGGSGLGGS

SEQ ID NO: 76 - protein, 10 aa, synthetic construct
GGLGSGGLGS

SEQ ID NO: 77 - protein, 10 aa, synthetic construct
GGGLSGGGLS

SEQ ID NO: 78 - protein, 10 aa, synthetic construct
GGGGLGGGGL

SEQ ID NO: 79 - protein, 10 aa, synthetic construct
LGGSGLGGSG

SEQ ID NO: 80 - protein, 10 aa, synthetic construct
GLGSGGLGSG

SEQ ID NO: 81 - protein, 10 aa, synthetic construct
GGLSGGGLSG

SEQ ID NO: 82 - protein, 10 aa, synthetic construct
GGGLGGGGLG

SEQ ID NO: 83 - protein, 10 aa, synthetic construct
GGGSLGGGSL

SEQ ID NO: 84 - protein, 10 aa, synthetic construct
LGGSSLGGSS

SEQ ID NO: 85 - protein, 10 aa, synthetic construct
GLGSSGLGSS

SEQ ID NO: 86 - protein, 10 aa, synthetic construct
GGLSSGGLSS

SEQ ID NO: 87 - protein, 6 aa, synthetic construct
GSGGGA

SEQ ID NO: 88 - protein, 12 aa, synthetic construct
GSGGGAGSGGGA

SEQ ID NO: 89 - protein, 18 aa, synthetic construct
GSGGGAGSGGGAGSGGGA

SEQ ID NO: 90 - protein, 24 aa, synthetic construct
GSGGGAGSGGGAGSGGGAGSGGGA

SEQ ID NO: 91 - protein, 10 aa, synthetic construct
GENLYFQSGG

SEQ ID NO: 92 - protein, 10 aa, synthetic construct
SGGGSSGGGS

SEQ ID NO: 93 - protein, 10 aa, synthetic construct
SSGGGSSGGG

SEQ ID NO: 94 - protein, 10 aa, synthetic construct
GGSGGGGSGG

SEQ ID NO: 95 - protein, 10 aa, synthetic construct
GSGSGSGSGS

SEQ ID NO: 96 - protein, 6 aa, synthetic construct
GGGSSS

SEQ ID NO: 97 - protein, 15 aa, synthetic construct
GGGSSGGGSSGGGSS

SEQ ID NO: 98 - protein, 8 aa, synthetic construct
GLGGLAAA

SEQ ID NO: 99 - protein, 12 aa, synthetic construct
KPEPKPAPAPKP

SEQ ID NO: 100 - protein, 12 aa, synthetic construct
AEAAAKEAAAKA

SEQ ID NO: 101 - protein, 5 aa, synthetic construct
  REPEAT 1..5: note = m
EAAAK

SEQ ID NO: 102 - protein, 15 aa, synthetic construct
PSRLEEELRRRLTEP

SEQ ID NO: 103 - protein, 8 aa, synthetic construct
SACYCELS

SEQ ID NO: 104 - protein, 16 aa, synthetic construct
TQKSLSLSPGKGLGGL

SEQ ID NO: 105 - protein, 13 aa, synthetic construct
SLSLSPGKGLGGL

SEQ ID NO: 106 - protein, 36 aa, synthetic construct
GGSAGGSGSGSSGGSSGASGTGTAGGTGSGSGTGSG

SEQ ID NO: 107 - protein, 36 aa, synthetic construct
GGSGGGSEGGGSEGGGSEGGGSEGGGSEGGGSGGGS

SEQ ID NO: 108 - protein, 12 aa, synthetic construct
ELKTPLGDTTHT

SEQ ID NO: 109 - protein, 15 aa, synthetic construct
EPKSCDTPPPCPRCP

SEQ ID NO: 110 - protein, 5 aa, synthetic construct
GLSGL

SEQ ID NO: 111 - protein, 5 aa, synthetic construct
EAAAK

SEQ ID NO: 112 - protein, 7 aa, synthetic construct
  REPEAT 1..5: note = m
EAAAKGS

SEQ ID NO: 113 - protein, 6 aa, synthetic construct
  REPEAT 1..4: note = m
EAAKGS

SEQ ID NO: 114 - protein, 17 aa, synthetic construct
GPSRLEEELRRRLTEPG

SEQ ID NO: 115 - protein, 12 aa, synthetic construct
HEYGAEALERAG

SEQ ID NO: 116 - protein, 22 aa, Homo sapiens
MQVSTAALAVLLCTMALCNQFS

SEQ ID NO: 117 - protein, 23 aa, Homo sapiens
MQVSTAALAVLLCTMALCNQVLS

SEQ ID NO: 118 - protein, 501 aa, synthetic construct [sequence omitted]

SEQ ID NO: 119 - protein, 501 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 120 - protein, 259 aa, synthetic construct
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 121 - protein, 465 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 122 - protein, 469 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPEA

SEQ ID NO: 123 - protein, 465 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 124 - protein, 469 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPEA

SEQ ID NO: 125 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 126 - protein, 4 aa, synthetic construct
EPEA

SEQ ID NO: 127 - protein, 602 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKIYEFDYHLYGQNITMIMTSVSGHLLAGGGGSGGGGSAEYGDYQPEVHGVPYFRLEHYLPARVMGGGGSGGGGSGSLFGSSRVQYVVNPAVKIVFLNIDPSGGGGSGGGGSLWVYLRPVPRPATIYLQILRLKPLTGEGGGGSGGGGSTLAFLVLSTPAMFNRALKPFLKSCHFQGGGGSGGGGSFVSPMAHYVPGIMAIESVVARFQFIVPGGGGSGGGGSVILPQAPSGPSYATYLQPAQAQMLTPPGGGGSGGGGSEVIQTSKYYMRDVIAIESAWLLELAPHGGGGSGGGGSDTLSAMSNPRAMQVLLQIQQGLQTLATGGGGSGGGGSKSWIHCWKYLSVQSQLFRGSSLLFRRV

SEQ ID NO: 128 - protein, 602 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKIYEFDYHLYGQNITMIMTSVSGHLLAGGGGSGGGGSAEYGDYQPEVHGVPYFRLEHYLPARVMGGGGSGGGGSGSLFGSSRVQYVVNPAVKIVFLNIDPSGGGGSGGGGSLWVYLRPVPRPATIYLQILRLKPLTGEGGGGSGGGGSTLAFLVLSTPAMFNRALKPFLKSCHFQGGGGSGGGGSFVSPMAHYVPGIMAIESVVARFQFIVPGGGGSGGGGSVILPQAPSGPSYATYLQPAQAQMLTPPGGGGSGGGGSEVIQTSKYYMRDVIAIESAWLLELAPHGGGGSGGGGSDTLSAMSNPRAMQVLLQIQQGLQTLATGGGGSGGGGSKSWIHCWKYLSVQSQLFRGSSLLFRRV

SEQ ID NO: 129 - protein, 27 aa, unidentified
VILPQAPSGPSYATYLQPAQAQMLTPP

SEQ ID NO: 130 - protein, 27 aa, unidentified
DTLSAMSNPRAMQVLLQIQQGLQTLAT

SEQ ID NO: 131 - protein, 27 aa, unidentified
EVIQTSKYYMRDVIAIESAWLLELAPH

SEQ ID NO: 132 - protein, 27 aa, unidentified
GSLFGSSRVQYVVNPAVKIVFLNIDPS

SEQ ID NO: 133 - protein, 27 aa, unidentified
AEYGDYQPEVHGVPYFRLEHYLPARVM

SEQ ID NO: 134 - protein, 27 aa, unidentified
KSWIHCWKYLSVQSQLFRGSSLLFRRV

SEQ ID NO: 135 - protein, 27 aa, unidentified
TLAFLVLSTPAMFNRALKPFLKSCHFQ

SEQ ID NO: 136 - protein, 27 aa, unidentified
FVSPMAHYVPGIMAIESVVARFQFIVP

SEQ ID NO: 137 - protein, 27 aa, unidentified
LWVYLRPVPRPATIYLQILRLKPLTGE

SEQ ID NO: 138 - protein, 27 aa, unidentified
KIYEFDYHLYGQNITMIMTSVSGHLLA

SEQ ID NO: 139 - DNA, 210 bp, synthetic construct
  source 1..210: mol_type = other DNA
gcaccacttgctgctgacacgccgaccgcctgctgcttcagctacacctcccgacagattccacagaatttcatagctgcttactttgagacgagcagccagtgctccaagcccagtgtcatcttcctaaccaagagaggccggcaggtctgtgctgaccccagtgaggagtgggtccagaaatacgtcagtgacctggagctgagtgcc

SEQ ID NO: 140 - protein, 70 aa, synthetic construct
ASLAADTATACCFSYTSRQIPQNFIAAYFETSSQCSKPGVIFLTKRSRQVCADPSEEWVQKYVSDLALSA

SEQ ID NO: 141 - protein, 70 aa, synthetic construct
APLAADTATACCFSYTSRQIPQNFIAAYFETSSQCSKPSVIFLTKRGRQVCADPSEEWVQKYVSDLALSA

SEQ ID NO: 142 - protein, 501 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKPGGGSSGGGSGMFQDPQERPRKLPQLCTELQTTIHDIILECVYCKQQLLRREVYDFARRDLCIVYRDGNPYAVRDKCLKFYSKISEYRHYCYSLYGTTLEQQYNKPLCDLLIRCINRQKPLCPEEKQRHLDKKQRFHNIRGRWTGRCMSCCRSSRTRRETQL

SEQ ID NO: 143 - protein, 9 aa, unidentified
RAHYNIVTF

SEQ ID NO: 144 - protein, 15 aa, unidentified
MFQDPQERPRKLPQL

SEQ ID NO: 145 - protein, 15 aa, unidentified
RPRKLPQLCTELQTT

SEQ ID NO: 146 - protein, 15 aa, unidentified
LCTELQTTIHDIILE

SEQ ID NO: 147 - protein, 15 aa, unidentified
TIHDIILECVYCKQQ

SEQ ID NO: 148 - protein, 15 aa, unidentified
ECVYCKQQLLRREVY

SEQ ID NO: 149 - protein, 15 aa, unidentified
QLLRREVYDFARRDL

SEQ ID NO: 150 - protein, 15 aa, unidentified
YDFARRDLCIVYRDG

SEQ ID NO: 151 - protein, 15 aa, unidentified
LCIVYRDGNPYAVRD

SEQ ID NO: 152 - protein, 15 aa, unidentified
GNPYAVRDKCLKFYS

SEQ ID NO: 153 - protein, 15 aa, unidentified
DKCLKFYSKISEYRH

SEQ ID NO: 154 - protein, 465 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 155 - protein, 972 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVDFTGSNGDPSSPYSLHYLSPTGVNEYGGGGSGGGGSGEKRSISAIRSYQYVMKICAAHLPTESGGGGSGGGGSYMVRFKDTKAQFSFFNPKCLLPTSRHYGGGGSGGGGSAEPESPEKEQAYLGYLAMLEELKKQEAGGGGSGGGGSESEGLHEVLAFAILKSDMDLRRTLFSNGGGGSGGGGSSKLLSFMAPIDHTTMSDDARTELFRSLGGGGSGGGGSSMDIDPSSSVLFEYMEKPDFSLFSPTMGGGGSGGGGSSLVISASIIVFNLLELEGDYRDDHIFSGGGGSGGGGSNGRVLELFRAAQLANDVVLQIMELCGAGGGGSGGGGSKARDETAALLNSAVLGAAPLFVPPADCGGGGSGGGGSLSNRILWIGIANFQLCPLILIWQILYAGGGGSGGGGSGIPVHLELASMTNMELMSSIVHQQVFPGGGGSGGGGSTLPLQPFQLAFGHLVNRQVFRQGPQPSGGGGSGGGGSITTDVLYTICNPCVPVQRIVIFRKNGVGGGGSGGGGSHVLPKCKHEFCTSSISKAMLIKPVCPVGGGGSGGGGSRGGGLLKYCHLLVLGFRPRPSTDVRALGGGGSGGGGSRILKAGGKILTFDRLALESPKGRGTVLGGGGSGGGGSNNRVAVATINFRRLVCPQEDKTSTDVLGGGGSGGGGSVCKACDKSFHFYCPLKVHMKRCRVAKSGGGGSGGGGSSSSAYTGYVERSPLAASTYSLDILYSG

SEQ ID NO: 156 - protein, 972 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVDFTGSNGDPSSPYSLHYLSPTGVNEYGGGGSGGGGSGEKRSISAIRSYQYVMKICAAHLPTESGGGGSGGGGSYMVRFKDTKAQFSFFNPKCLLPTSRHYGGGGSGGGGSAEPESPEKEQAYLGYLAMLEELKKQEAGGGGSGGGGSESEGLHEVLAFAILKSDMDLRRTLFSNGGGGSGGGGSSKLLSFMAPIDHTTMSDDARTELFRSLGGGGSGGGGSSMDIDPSSSVLFEYMEKPDFSLFSPTMGGGGSGGGGSSLVISASIIVFNLLELEGDYRDDHIFSGGGGSGGGGSNGRVLELFRAAQLANDVVLQIMELCGAGGGGSGGGGSKARDETAALLNSAVLGAAPLFVPPADCGGGGSGGGGSLSNRILWIGIANFQLCPLILIWQILYAGGGGSGGGGSGIPVHLELASMTNMELMSSIVHQQVFPGGGGSGGGGSTLPLQPFQLAFGHLVNRQVFRQGPQPSGGGGSGGGGSITTDVLYTICNPCVPVQRIVIFRKNGVGGGGSGGGGSHVLPKCKHEFCTSSISKAMLIKPVCPVGGGGSGGGGSRGGGLLKYCHLLVLGFRPRPSTDVRALGGGGSGGGGSRILKAGGKILTFDRLALESPKGRGTVLGGGGSGGGGSNNRVAVATINFRRLVCPQEDKTSTDVLGGGGSGGGGSVCKACDKSFHFYCPLKVHMKRCRVAKSGGGGSGGGGSSSSAYTGYVERSPLAASTYSLDILYSG

SEQ ID NO: 157 - protein, 972 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVDFTGSNGDPSSPYSLHYLSPTGVNEYGGGGSGGGGSGEKRSISAIRSYQYVMKICAAHLPTESGGGGSGGGGSYMVRFKDTKAQFSFFNPKCLLPTSRHYGGGGSGGGGSAEPESPEKEQAYLGYLAMLEELKKQEAGGGGSGGGGSESEGLHEVLAFAILKSDMDLRRTLFSNGGGGSGGGGSSKLLSFMAPIDHTTMSDDARTELFRSLGGGGSGGGGSSMDIDPSSSVLFEYMEKPDFSLFSPTMGGGGSGGGGSSLVISASIIVFNLLELEGDYRDDHIFSGGGGSGGGGSNGRVLELFRAAQLANDVVLQIMELCGAGGGGSGGGGSKARDETAALLNSAVLGAAPLFVPPADCGGGGSGGGGSLSNRILWIGIANFQLCPLILIWQILYAGGGGSGGGGSGIPVHLELASMTNMELMSSIVHQQVFPGGGGSGGGGSTLPLQPFQLAFGHLVNRQVFRQGPQPSGGGGSGGGGSITTDVLYTICNPCVPVQRIVIFRKNGVGGGGSGGGGSHVLPKCKHEFCTSSISKAMLIKPVCPVGGGGSGGGGSRGGGLLKYCHLLVLGFRPRPSTDVRALGGGGSGGGGSRILKAGGKILTFDRLALESPKGRGTVLGGGGSGGGGSNNRVAVATINFRRLVCPQEDKTSTDVLGGGGSGGGGSVCKACDKSFHFYCPLKVHMKRCRVAKSGGGGSGGGGSSSSAYTGYVERSPLAASTYSLDILYSG

SEQ ID NO: 158 - protein, 972 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVDFTGSNGDPSSPYSLHYLSPTGVNEYGGGGSGGGGSGEKRSISAIRSYQYVMKICAAHLPTESGGGGSGGGGSYMVRFKDTKAQFSFFNPKCLLPTSRHYGGGGSGGGGSAEPESPEKEQAYLGYLAMLEELKKQEAGGGGSGGGGSESEGLHEVLAFAILKSDMDLRRTLFSNGGGGSGGGGSSKLLSFMAPIDHTTMSDDARTELFRSLGGGGSGGGGSSMDIDPSSSVLFEYMEKPDFSLFSPTMGGGGSGGGGSSLVISASIIVFNLLELEGDYRDDHIFSGGGGSGGGGSNGRVLELFRAAQLANDVVLQIMELCGAGGGGSGGGGSKARDETAALLNSAVLGAAPLFVPPADCGGGGSGGGGSLSNRILWIGIANFQLCPLILIWQILYAGGGGSGGGGSGIPVHLELASMTNMELMSSIVHQQVFPGGGGSGGGGSTLPLQPFQLAFGHLVNRQVFRQGPQPSGGGGSGGGGSITTDVLYTICNPCVPVQRIVIFRKNGVGGGGSGGGGSHVLPKCKHEFCTSSISKAMLIKPVCPVGGGGSGGGGSRGGGLLKYCHLLVLGFRPRPSTDVRALGGGGSGGGGSRILKAGGKILTFDRLALESPKGRGTVLGGGGSGGGGSNNRVAVATINFRRLVCPQEDKTSTDVLGGGGSGGGGSVCKACDKSFHFYCPLKVHMKRCRVAKSGGGGSGGGGSSSSAYTGYVERSPLAASTYSLDILYSG

SEQ ID NO: 159 - protein, 27 aa, unidentified
KARDETAALLNSAVLGAAPLFVPPADC

SEQ ID NO: 160 - protein, 27 aa, unidentified
LSNRILWIGIANFQLCPLILIWQILYA

SEQ ID NO: 161 - protein, 27 aa, unidentified
NGRVLELFRAAQLANDVVLQIMELCGA

SEQ ID NO: 162 - protein, 27 aa, unidentified
GIPVHLELASMTNMELMSSIVHQQVFP

SEQ ID NO: 163 - protein, 27 aa, unidentified
NNRVAVATINFRRLVCPQEDKTSTDVL

SEQ ID NO: 164 - protein, 27 aa, unidentified
AEPESPEKEQAYLGYLAMLEELKKQEA

SEQ ID NO: 165 - protein, 27 aa, unidentified
ESEGLHEVLAFAILKSDMDLRRTLFSN

SEQ ID NO: 166 - protein, 27 aa, unidentified
TLPLQPFQLAFGHLVNRQVFRQGPQPS

SEQ ID NO: 167 - protein, 27 aa, unidentified
YMVRFKDTKAQFSFFNPKCLLPTSRHY

SEQ ID NO: 168 - protein, 27 aa, unidentified
RILKAGGKILTFDRLALESPKGRGTVL

SEQ ID NO: 169 - protein, 27 aa, unidentified
VCKACDKSFHFYCPLKVHMKRCRVAKS

SEQ ID NO: 170 - protein, 27 aa, unidentified
RGGGLLKYCHLLVLGFRPRPSTDVRAL

SEQ ID NO: 171 - protein, 27 aa, unidentified
SKLLSFMAPIDHTTMSDDARTELFRSL

SEQ ID NO: 172 - protein, 27 aa, unidentified
GEKRSISAIRSYQYVMKICAAHLPTES

SEQ ID NO: 173 - protein, 27 aa, unidentified
SLVISASIIVFNLLELEGDYRDDHIFS

SEQ ID NO: 174 - protein, 27 aa, unidentified
HVLPKCKHEFCTSSISKAMLIKPVCPV

SEQ ID NO: 175 - protein, 27 aa, unidentified
VDFTGSNGDPSSPYSLHYLSPTGVNEY

SEQ ID NO: 176 - protein, 27 aa, unidentified
SMDIDPSSSVLFEYMEKPDFSLFSPTM

SEQ ID NO: 177 - protein, 27 aa, unidentified
SSSAYTGYVERSPLAASTYSLDILYSG

SEQ ID NO: 178 - protein, 27 aa, unidentified
ITTDVLYTICNPCVPVQRIVIFRKNGV

SEQ ID NO: 179 - protein, 237 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 180 - protein, 237 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 181 - protein, 237 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 182 - protein, 237 aa, synthetic construct
MQVSTAALAVLLCTMALCNQVLSAPLAADTPTACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 183 - protein, 237 aa, synthetic construct
MQVSTAALAVLLCTMALCNQVLSAPLAADTATACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 184 - protein, 491 aa, unidentified
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLENLYFQSMLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 185 - protein, 491 aa, synthetic construct
MQVSTAALAVLLCTMALCNQVLSAPLAADTATACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLENLYFQSMLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 186 - protein, 249 aa, synthetic construct
ENLYFQSMLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 187 - protein, 972 aa, synthetic construct
MQVSTAALAVLLCTMALCNQVLSAPLAADTATACCFSYTSRQIPQNFIAAYFETSSQCSKPSVIFLTKRGRQVCADPSEEWVQKYVSDLALSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLVDFTGSNGDPSSPYSLHYLSPTGVNEYGGGGSGGGGSGEKRSISAIRSYQYVMKICAAHLPTESGGGGSGGGGSYMVRFKDTKAQFSFFNPKCLLPTSRHYGGGGSGGGGSAEPESPEKEQAYLGYLAMLEELKKQEAGGGGSGGGGSESEGLHEVLAFAILKSDMDLRRTLFSNGGGGSGGGGSSKLLSFMAPIDHTTMSDDARTELFRSLGGGGSGGGGSSMDIDPSSSVLFEYMEKPDFSLFSPTMGGGGSGGGGSSLVISASIIVFNLLELEGDYRDDHIFSGGGGSGGGGSNGRVLELFRAAQLANDVVLQIMELCGAGGGGSGGGGSKARDETAALLNSAVLGAAPLFVPPADCGGGGSGGGGSLSNRILWIGIANFQLCPLILIWQILYAGGGGSGGGGSGIPVHLELASMTNMELMSSIVHQQVFPGGGGSGGGGSTLPLQPFQLAFGHLVNRQVFRQGPQPSGGGGSGGGGSITTDVLYTICNPCVPVQRIVIFRKNGVGGGGSGGGGSHVLPKCKHEFCTSSISKAMLIKPVCPVGGGGSGGGGSRGGGLLKYCHLLVLGFRPRPSTDVRALGGGGSGGGGSRILKAGGKILTFDRLALESPKGRGTVLGGGGSGGGGSNNRVAVATINFRRLVCPQEDKTSTDVLGGGGSGGGGSVCKACDKSFHFYCPLKVHMKRCRVAKSGGGGSGGGGSSSSAYTGYVERSPLAASTYSLDILYSG

SEQ ID NO: 188 - DNA, 210 bp, synthetic construct
  source 1..210: mol_type = other DNA
gcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggccctgagtgcc

SEQ ID NO: 189 - DNA, 210 bp, synthetic construct
  source 1..210: mol_type = other DNA
gcaccacttgctgctgacacgccgaccgcctgctgcttcagctacacctcccgacagattccacagaatttcatagctgcttactttgagacgagcagccagtgctccaagcccagtgtcatcttcctaaccaagagaggccggcaggtctgtgctgaccccagtgaggagtgggtccagaaatacgtcagtgacctggccctgagtgcc

SEQ ID NO: 190 - DNA, 210 bp, synthetic construct
  source 1..210: mol_type = other DNA
gcaccacttgctgctgacacggccaccgcctgctgcttcagctacacctcccgacagattccacagaatttcatagctgcttactttgagacgagcagccagtgctccaagcccagtgtcatcttcctaaccaagagaggccggcaggtctgtgctgaccccagtgaggagtgggtccagaaatacgtcagtgacctggccctgagtgcc

(19) United States
(12) Patent Application Publication
(10) Pub. No.: US 2026/0345753 A1
(43) Pub. Date: Nov. 5, 2026

(54) Constructs And Their Use

(71) Applicant: Nykode Therapeutics ASA, Oslo (NO)

(72) Inventors: Joel Benjamin Heim, Oslo (NO); Stephanie Meryl Monson, South San Francisco, CA (US); Emily Crane Freund, South San Francisco, CA (US)

(73) Assignee: Nykode Therapeutics ASA, Oslo (NO)

(21) Appl. No.: 19/124,161

(22) PCT Filed: Oct. 25, 2023
(86) PCT No.: PCT/US2023/077759

§ 371 Date: Apr. 24, 2025

Related U.S. Application Data
(60) Provisional application No. 63/380,864, filed on Oct. 25, 2022.

(51) Int. Cl. A61K 39/00; A61K 39/215; A61P 31/14; A61P 31/20; C07K 14/52; C07K 14/715

(52) U.S. Cl. CPC A61K 39/0011; A61K 39/215; A61P 31/14; A61P 31/20; C07K 14/523; C07K 14/7158; A61K 2039/53; A61K 2039/585; A61K 2039/6031; A61K 2039/70; C07K 2319/02; C12N 2710/20034; C12N 2770/20034

(57) ABSTRACT

The present invention relates to constructs, i.e. multimeric proteins, polypeptides and polynucleotides encoding same, to methods for preparing such constructs, to pharmaceutical compositions comprising the constructs, and to the use of such pharmaceutical compositions in the treatment or prevention of diseases

### TECHNICAL FIELD

The present invention relates to constructs, i.e. multimeric proteins, polypeptides and polynucleotides encoding same, to methods for preparing such constructs, to pharmaceutical compositions comprising the constructs, and to the use of such pharmaceutical compositions in the treatment or prevention of diseases.

### RELATED APPLICATIONS

This application claims priority to U.S. Provisional No. 63/380,864, filed on Oct. 25, 2022, the content of which is incorporated herein by reference in its entirety.

### REFERENCE TO A SEQUENCE LISTING SUBMITTED ELECTRONICALLY AS AN XML FILE

The instant application contains a Sequence Listing which has been submitted in xml format and is hereby incorporated by reference in its entirety. Said xml copy, created on Oct. 24, 2023, is named P6501PC00—Sequence Listing.xml, and is 179 KB in size.

### BACKGROUND

Both B cell (humoral/antibody mediated) and T cell responses are important components of protective responses against infections caused by pathogens. Specific antibodies against pathogen antigens can mediate a broad range of effector functions, such as e.g. a) direct neutralization of toxins or pathogens, b) neutralization of pathogen virulence factors, c) binding to and trapping of pathogens in mucins, d) activating complement to mediate anti-pathogen phagocytic clearance, degradation or lysis, e) activating neutrophil opsonophagocytosis, f) inducing macrophage opsonophagocytosis g) activating natural killer (NK) cell degranulation to kill infected cells, h) enhancing antigen update, processing and presentation by dendritic cells to T and B cells, i) inducing degranulation of mast cells, basophils and eosinophils in the setting of parasitic infections (L. Lu et al., Nat Rev Immunol 18(1), 2018, 46).

Complementing these activities, T cell responses are critical for limiting viral replication and infection by killing the infected cells, inducing apoptosis, releasing antiviral substances, and/or inducing increased intracellular lysis in already infected cells and thus help to prevent, reduce severity of or cure the disease. In addition, effective and long-lasting response in both arms of immunity usually requires additional support from T-helper (Th1 and Th2) lymphocytes.

Cytotoxic T lymphocytes (CTL) also play a significant role (F. Sheperd et al., Int J Mol Sci 21, 2020, 6144) with e.g. intracellular pathogens where MHC class I-restricted CD8+ T cells are critical for clearing bacterial infections and are known to provide protective immunity against a range of bacterial species. MHC class II restricted CD4+ T cells support memory CD8+ T cell responses and are important for protective immunity against bacterial infections. Naïve CD4+ T cells differentiate subsets of cells with effector capacity, such as T helper 1 (Th1) and Th2 cells. After binding specific T cell epitopes on the surface of antigen-presenting cells (APCs), Th1 and Th2 cells supply specific soluble cytokine signals that regulate the balance between antibody and CTL immunity. Thus, effective immunity involves multiple antigen recognition events of specific pathogen immunogenic determinants (epitopes) by T-helper cells followed by molecular recognition by B cells, CTL, or both.

Different types of lymphocytes (B cells, CTLs and Th cells) specifically recognize different types of epitopes of the pathogen. B cell epitopes can be categorized as linear or conformational epitopes, with linear epitopes often being parts of conformational B-cell epitopes in native proteins. Conformational epitopes are exposed structural features on the surface of pathogens such as a viral envelope, bacterial outer membrane or secreted bacterial toxins. T cell epitopes are short peptides from any protein of a pathogen, which only have to conform to the host antigen-processing and MHC binding mechanisms, most notably class I or class II MHC haplotype restriction mechanisms. Suitable T cell epitopes occur with an estimated frequency of about one per 200-500 amino acid sequence, depending on host population and pathogen.

Vaccines against pathogens comprise the pathogen or parts thereof, modified in such a manner that no harm or disease is caused, but ensuring that when the host is confronted with that infectious agent, the immune system can adequately neutralize it before it causes any ill effect. For more than a hundred years, vaccination has been performed by one of two approaches: either introducing specific antigens against which the immune system reacts directly; or introducing live attenuated infectious agents that replicate within the host without causing disease and synthesize the antigens that subsequently prime the immune system.

Recently, a radically new approach to vaccination has been developed. Polynucleotide sequences (DNA or RNA), encoding antigen(s) capable of stimulating an immune response, are directly introduced into appropriate tissues for in situ production of the target antigen. This approach offers a number of potential advantages over traditional approaches, including the stimulation of both B- and T-cell responses, improved vaccine stability, the absence of any infectious agent and the relative ease of large-scale manufacture.

Although treatment of cancer has been improved over the past few decades, particularly due to early detection and diagnosis, which has significantly increased the survival of cancer patients, only about 60% of such patients are alive 5 years after the diagnosis. Most of the cancer treatments in use are surgical procedures, radiation and cytotoxic chemotherapeutics, however they all have serious side effects. Recently, also treatments using antibodies directed towards known cancer associated antigens are used.

Within the last few years, cancer immunotherapies targeting cancer cells with the help of the patient's own immune system, i.e. cancer vaccines, have attracted interest. Such therapies may reduce or even eliminate some of the side-effects associated with traditional cancer treatments.

There is thus a need for efficient agents and medicaments which can be used both for treating or preventing an infectious disease, and for treating or preventing cancer.

The Vaccibody construct is a multimeric fusion protein consisting of multiple polypeptides, each comprising a targeting unit that targets antigen-presenting cells, a multimerization unit and an antigenic unit which comprises one or more antigens or parts thereof (e.g. epitopes), and which, after administration to a subject, has shown to be efficient in generating an immune response against the antigens or parts thereof comprised in the antigenic unit. In some embodiments, the Vaccibody construct is a dimeric fusion protein consisting of two polypeptides, each comprising a targeting unit, which targets antigen-presenting cells, a dimerization unit and an antigenic unit.

The Vaccibody construct may be administered to a subject in the form of a polynucleotide encoding the polypeptide, e.g. a polynucleotide comprised in a vector, such as a DNA plasmid. After administration to host cells, e.g. administration to muscle cells of a subject, a polypeptide is expressed which, due to the multimerization unit, such as dimerization unit, forms a multimeric fusion protein, such as a dimer.

Vaccibodies having a human CCL3L1 targeting unit (SEQ ID NO: 2) have shown to trigger an efficient immune response, once administered to a subject, even for weak antigens, see WO 2011/161244A1.

The CCL3 gene family in humans is composed of four genes, all located on Chr17q21: CCL3 which encodes hCCL3 (also denoted LD78α or MIP-1α), CCL3L1, which encodes hCCL3L1 (also denoted LD78β), CCL3L2 and CCL3L3. Human CCL3 binds to various receptors, including CCR1, CCR4 and CCR5, while human CCL3L1 binds to CCR1, CCR3 and CCR5. These receptors are, inter alia, located on the surface of antigen presenting cells (APCs).

It is known that human MIP-1α and human MIP-1β can form in solution under physiologically relevant concentrations high-molecular weight aggregates, e.g. polymers that contain more than 50 monomers. While aggregation inhibits the binding of MIP-1 to its cognate receptors—since the monomer form is presumed to be the form that binds to the receptor and activates it—it also protects the protein from proteolysis (degradation by proteases), thus increasing its half-life and thereby extending the effective range of the chemotactic gradient of MIP-1. The aggregation is dynamic and reversible, and plays an important role in the regulation of the MIP-1 monomer. Introduction of depolymerisation mutations in the sequences of human MIP-1α and human MIP-1β decrease their oligomerization state relative to that of the wild types (see for instance M. Ren et al., EMBO J 2010(29), 3952-3966).

### SUMMARY

In a first aspect, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more antigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

In the polypeptide disclosed herein, the amino acid sequence of the targeting unit comprises one or more mutations, compared to the amino acid sequence of the wild type, which reduce or prevent the formation of targeting unit oligomers. Consequently, the nucleotide sequence of the polynucleotide which encodes the targeting unit encodes such mutation.

Alternatively, in a first aspect, the disclosure relates to a construct, the construct being:
(i) a polypeptide comprising a targeting unit selected from the group consisting of mutated human CCL3 and mutated human CCL3L1 and an antigenic unit comprising one or more antigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i); In some other embodiments, the disclosure relates to a construct, the construct being:
(i) a polynucleotide comprising a nucleotide sequence encoding a targeting unit selected from the group consisting of mutated human CCL3 and mutated human CCL3L1, a dimerization unit and an antigenic unit comprising one or more antigens or parts thereof; or
(ii) a polypeptide encoded by the nucleotide sequence as defined in (i); or
(iii) a dimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some other embodiments, the disclosure relates to a construct, the construct being:
(i) a polypeptide comprising a targeting unit selected from the group consisting of mutated human CCL3 and mutated human CCL3L1, a dimerization unit and an antigenic unit comprising one or more antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a dimeric protein consisting of multiple polypeptides as defined in (i);

In another aspect, the disclosure relates to a vector comprising the polynucleotide as defined herein.

In some embodiments, the vector additionally comprises one or more nucleotide sequences encoding one or more further polypeptides, and the vector allows for the co-expression of the polypeptide and the one or more further polypeptides as separate molecules, i.e., the vector is a polycistronic vector.

In yet another aspect, the disclosure relates to a host cell comprising the vector or polynucleotide as defined herein.

In yet another aspect, the disclosure provides a multimeric protein consisting of multiple polypeptides as defined herein. In some embodiments, the disclosure provides a dimeric protein consisting of two polypeptides as defined herein.

In yet another aspect, the disclosure provides methods for preparing the construct, polypeptide or multimeric protein as defined herein.

In yet another aspect, the disclosure provides the construct, the polynucleotide, the vector, the polypeptide or the multimeric protein as defined herein, for use as a medicament.

In yet another aspect, the disclosure provides a pharmaceutical composition comprising the construct, the polynucleotide, the vector, the polypeptide or the multimeric protein as defined herein, and a pharmaceutically acceptable carrier.

In yet another aspect, the disclosure provides methods for preparing the pharmaceutical composition.

In yet another aspect, the disclosure provides methods for the prophylactic or therapeutic treatment of a disease, e.g. for the therapeutic treatment of cancer or for the prophylactic or therapeutic treatment of an infectious disease, wherein the pharmaceutical composition is administered to a subject in need of such prophylactic or therapeutic treatment and/or the use of the pharmaceutical composition for the prophylactic or therapeutic treatment of a disease, e.g. for the therapeutic treatment of cancer or for the prophylactic or therapeutic treatment of an infectious disease, by administering the composition to a subject in need of such prophylactic or therapeutic treatment.

### DESCRIPTION OF DRAWINGS

The following abbreviations are used in the description of the drawings:
Ab: antibody
a: anti such as a-human, a-goat, a-mouse, a-rabbit, a-SARS CoV2 and the like: anti-human, anti-goat, anti-mouse, anti-rabbit, anti-SARS-CoV2.

FIG. 1 Schematic drawing of a polypeptide

Shows an embodiment of a polypeptide as disclosed herein which is described in more detail in the section “Constructs” herein.

FIG. 2: Expression and secretion levels of proteins encoded by DNA plasmids

Shows the expression and secretion of proteins encoded by DNA plasmids VB1020 and TECH011-CV006 detected in the supernatants of Expi293F cells transfected with said DNA plasmids by ELISA. Supernatants of cells transfected with VB1020 and TECH011-CV006 were diluted 1:50 and 1:1500, respectively, and ELISA was performed using mouse a-human IgG CH3 domain capture Ab (MCA878G) and a-human MIP-1α biotinylated detection Ab (BAF270). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 3: CCR5 receptor activation by proteins encoded by DNA plasmids

Shows the activation of hCCR5 (CCR5-bla U2OS reporter cell line) by proteins encoded by DNA plasmids VB1020, and TECH011-CV006 using supernatants of Expi293F cells transfected with said DNA plasmids. The fluorescence ratio at 460 and 530 nm detected upon beta-lactamase substrate addition and excitation at 409 nm, called the response ratio, provides a measure of the activation of hCCR5. 3 nM recombinant CCL3L1 (rCCL3L1) was used as a positive control. Transfection ctrl. (=cells only treated with transfection agent ExpiFectamine™ 293 reagent which serve as a negative control).

FIG. 4: Western Blot of proteins expressed from DNA plasmids

Western blot shows expression and secretion of intact proteins expressed from VB1020 and TECH011-CV006 by detecting the targeting unit in said proteins. Reduced supernatant samples from transfection control and cells transfected with VB1020 and TECH011-CV006, respectively, were run on an SDS-PAGE and proteins were transferred to a PVDF-membrane. The membrane was immunoblotted using primary antibody: goat a-human MIP-1α (AF270). Secondary antibody: donkey a-goat, Dylight 800 (SA5-10092). Chemidoc channels Dylight 800 and 650 (for protein standard).

FIG. 5: Western blot of proteins expressed from DNA plasmids

Western blot shows expression and secretion of intact proteins expressed from VB1020 and TECH011-CV006 by detecting the E6 antigen in the antigenic unit in said proteins. Reduced supernatant samples from transfection control and cells transfected with VB1020 and TECH011-CV006, respectively, were run on an SDS-PAGE and proteins were transferred to a PVDF-membrane. The membrane was immunoblotted using primary antibody: mouse a-E6 Papillomavirus Type 16 antibody (IG-E6-6F4). Secondary antibody: donkey a-mouse, Dylight 800 (SA5-10172). Chemidoc channels Dylight 800 and 650 (for protein standard).

FIG. 6: Expression and secretion levels of proteins encoded by DNA plasmids

Shows the expression and secretion of proteins encoded by DNA plasmids VB2060 and TECH011-IV002 detected in the supernatants of Expi293F cells transfected with said DNA plasmids by ELISA. Supernatants of cells transfected with VB2060 and TECH011-IV002 were diluted 1:2000 and 1:4000, respectively, and ELISA was performed using mouse a-human IgG CH3 domain capture Ab (MCA878G) and a-human MIP-1α biotinylated detection Ab (BAF270). Transfection control (=cells only treated with transfection agent ExpiFectamine™ 293 reagent which serve as a negative control). Protein concentration (in ng/mL) in the cell supernatant was interpolated from a standard curve of purified protein.

FIG. 7: CCR5 receptor activation by proteins encoded by DNA plasmids

Shows the activation of hCCR5 (CCR5-bla U2OS reporter cell line) by proteins encoded by DNA plasmids VB2060 and TECH011-IV002 using supernatants of Expi293F cells transfected with said DNA plasmids. The fluorescence ratio at 460 and 530 nm detected upon beta-lactamase substrate addition and excitation at 409 nm, called the response ratio, provides a measure of the activation of hCCR5. 3 nM recombinant CCL3L1 (rCCL3L1) was used as a positive control. Transfection control (=cells only treated with transfection agent ExpiFectamine™ 293 reagent which serve as a negative control).

FIG. 8: Western Blot of proteins expressed from DNA plasmids

Western blot shows expression and secretion of intact proteins expressed from VB2060 and TECH011-IV002 by detecting the targeting unit in said proteins. Reduced supernatant samples from transfection control and cells transfected with VB2060 and TECH011-IV002, respectively, were run on an SDS-PAGE and proteins were transferred to a PVDF-membrane. The membrane was immunoblotted using primary antibody: goat a-human MIP-1α (AF270). Secondary antibody: donkey a-goat, Dylight 800 (SA5-10092). Chemidoc channels Dylight 800 and 650 (for protein standard).

FIG. 9: Western Blot of proteins expressed from DNA plasmids

Western blot shows expression and secretion of intact proteins expressed from VB2060 and TECH011-IV002 by detecting the SARS-CoV-2 Spike receptor binding domain comprised in the antigenic unit in said protein. Reduced supernatant samples from transfection control and cells transfected with VB2060 and TECH011-IV002, respectively were run on an SDS-PAGE and proteins were transferred to a PVDF-membrane. The membrane was immunoblotted using primary antibody: rabbit a-SARS-CoV-2 (2019-nCoV) Spike RBD (40592-T62). Secondary antibody: donkey a-rabbit, Dylight 650 (SA5-10041). Chemidoc channel Dylight 650.

FIG. 10: Analysis of purified proteins encoded by DNA plasmids

SDS-PAGE of affinity purification of VB4231 (FIG. 10A) and TECH011-IV011 (FIG. 10B). Equilibrated CaptureSelect C-tagXL Affinity Matrix was incubated with supernatant of Expi293F cells transfected with VB4231 and TECH011-IV011, respectively and washed with Tris-buffered saline (TBS) and 25 mM Tris-HCl pH 7.2, 1 M NaCl, 0.05% Tween. Protein was eluted with 2 mM SEPEA peptide in TBS. SDS-PAGE was performed under reducing conditions with samples taken from each eluted fraction. Protein was detected with InstantBlue™ Coomassie Stain.

FIG. 11: Analysis of purified proteins encoded by DNA plasmids

SDS-PAGE of size exclusion chromatography-purified proteins expressed from VB4231 (FIG. 11A) and TECH011-IV011 (FIG. 11B) under reducing (red) and non-reducing (non-red) conditions. Proteins were loaded at 0.5 μg and 2 μg, respectively. Protein was detected with InstantBlue™ Coomassie Stain.

FIG. 12: Oligomerization state of proteins encoded by DNA plasmids

Analytical size exclusion chromatogram of purified proteins expressed from VB4231 (FIG. 12A) and TECH011-IV011 (FIG. 12B) shows reduced oligomerization for protein expressed from TECH011-IV011 compared to protein expressed from VB4231.

FIG. 13: Expression and secretion levels of proteins encoded by DNA plasmids

Shows the expression and secretion of protein encoded by DNA plasmids VB4097 and TECH011-CV007 detected in the supernatants of Expi293F cells transfected with said DNA plasmids by ELISA. ELISA was performed using mouse a-human IgG CH3 domain capture Ab (MCA878G) and a-human MIP-1α biotinylated detection Ab (BAF270). Transfection control (=cells only treated with transfection agent ExpiFectamine™ 293 reagent which serve as a negative control). Protein concentration (in ng/mL) in the cell supernatant was interpolated from a standard curve of purified protein.

FIG. 14: Receptor activation of proteins encoded by DNA plasmids

Shows the activation of hCCR5 (CCR5-bla U2OS reporter cell line) by proteins encoded by DNA plasmids VB4097 and TECH011-CV007 using supernatants of Expi293F cells transfected with said DNA plasmids. The fluorescence ratio at 460 and 530 nm detected upon beta-lactamase substrate addition and excitation at 409 nm, called the response ratio, provides a measure of the activation of hCCR5. 3 nM recombinant CCL3L1 (rCCL3L1) was used as a positive control. Transfection control (=cells only treated with transfection agent ExpiFectamine™ 293 reagent which serve as a negative control).

FIG. 15: Western Blot of proteins expressed from DNA plasmids

Western blot shows secretion of intact proteins expressed from VB4097 and TECH011-CV007 by detecting the targeting unit in said proteins. Reduced supernatant samples from transfection control and cells transfected with VB4097 and TECH011-CV007, respectively, were run on an SDS-PAGE and proteins transferred to a PVDF-membrane. The membrane was immunoblotted using primary antibody: goat a-human MIP-1α (AF270). Secondary antibody: donkey a-goat, Dylight 800 (SA5-10092). Chemidoc channels Dylight 800 and 650 (for protein standard).

FIG. 16: Expression and secretion levels of proteins encoded by DNA plasmids

Shows the expression and secretion of proteins encoded by DNA plasmids VB1020, TECH011-CV006 and TECH011-CV036 detected by ELISA in the supernatants of Expi293F cells transfected with said DNA plasmids. Supernatants of cells transfected with VB1020, TECH011-CV006 and TECH011-CV036 were harvested on day 3 post-transfection and diluted, and ELISA was performed using a mouse a-human IgG CH3 domain capture Ab (MCA878G, Bio-Rad) and an a-human MIP-1α biotinylated detection Ab (BAF270, R&D systems). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 17: Expression and secretion levels of proteins encoded by DNA plasmids

Shows the expression and secretion of proteins encoded by DNA plasmids VB1020, TECH011-CV006 and TECH011-CV036 detected by ELISA in the supernatants of Expi293F cells transfected with said DNA plasmids. Supernatants of cells transfected with VB1020, TECH011-CV006 and TECH011-CV036 were harvested on either day 1 or day 3 after transfection and diluted, and ELISA was performed using a mouse a-human IgG CH3 domain capture Ab (MCA878G, Bio-Rad) and an a-human MIP-1α biotinylated detection Ab (BAF270, R&D systems). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 18: Western Blot of proteins expressed from DNA plasmids

Western blot shows expression and secretion of intact proteins expressed from VB1020, TECH011-CV006 and TECH011-CV036 by detecting the targeting unit in said proteins. Reduced supernatant samples (harvested on day 3 after transfection) from transfection control and cells transfected with VB1020, TECH011-CV006 and TECH011-CV036, respectively, were run on an SDS-PAGE. Prior sample preparation, supernatant samples from cells transfected with TECH011-CV006 and TECH011-CV036 were diluted 1:10 in expression medium. The proteins were transferred to a PVDF-membrane. The membrane was immunoblotted using the primary antibody: goat a-human MIP-1α (AF270), and the secondary antibody: donkey a-goat, Dylight 550 (SA5-10087). Chemidoc channels Dylight 550 and 650 (for protein standard).

FIG. 19: Quantified Western Blot signal of proteins expressed from DNA plasmids

Shows quantification of the band intensities from the Western Blot shown in FIG. 18 Signal intensity was adjusted for the dilution of TECH011-CV006 and TECH011-CV036.

FIG. 20: Immunogenicity of DNA plasmids

Shows the immunogenicity of DNA plasmids VB1020 and TECH011-CV006 in mice administered with various doses of these plasmids by way of measuring IFN-γ secretion from T cells (total T cell response) compared to the negative control VB1026.

FIG. 21: Immunogenicity of DNA plasmids

Shows the immunogenicity of DNA plasmids VB1020 and TECH011-CV006 in mice administered with various doses of these plasmids byway of measuring TNF-α secretion from T cells (total T cell response) compared to the negative control VB1026.

FIG. 22: Immunogenicity of DNA plasmids

Shows the immunogenicity of DNA plasmids VB1020 and TECH011-CV006 in mice administered with various doses these plasmids by way of measuring IFN-γ and TNF-α secretion from T cells (total T cell response) compared to the negative control VB1026.

FIG. 23: Immunogenicity of DNA plasmids

Shows the immunogenicity of DNA plasmids VB1020 and TECH011-CV006 in mice administered with 1 μg dose of these plasmids by way of measuring IFN-γ and TNF-α secretion from T cells (total T cell response).

FIG. 24: Expression and secretion levels of proteins encoded by DNA plasmids

Shows the expression and secretion of proteins encoded by DNA plasmids VB2060, TECH011-IV002 and TECH011-IV015 detected by ELISA in the supernatants of Expi293F cells transfected with said DNA plasmids. Supernatants of cells transfected with VB2060, TECH011-IV002 and TECH011-IV015 were harvested on day 3 after transfection and diluted, and ELISA was performed using a mouse a-human IgG CH3 domain capture Ab (MCA878G) and a-human MIP-1α biotinylated detection Ab (BAF270). Transfection ctrl (=cells only treated with transfection agent ExpiFectamine™ 293 reagent which serve as a negative control). Protein concentration (in ng/mL) in the cell supernatant was interpolated from a standard curve of purified protein.

FIG. 25: Expression and secretion levels of proteins encoded by DNA plasmids

Shows the expression and secretion of proteins encoded by DNA plasmids VB2060, TECH011-IV002 and TECH011-IV015 detected by ELISA in the supernatants of Expi293F cells transfected with said DNA plasmids. Supernatants of cells transfected with VB2060, TECH011-IV002 and TECH011-IV015 were harvested on day 1 or on day 3 after transfection and diluted, and ELISA was performed using a mouse a-human IgG CH3 domain capture Ab (MCA878G) and an a-human MIP-1α biotinylated detection Ab (BAF270). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 26: Western Blot of proteins expressed from DNA plasmids

Western blot shows expression and secretion of intact proteins expressed from VB2060, TECH011-IV002 and TECH011-IV015 by detecting the targeting unit in said proteins. Reduced and non-reduced supernatant samples (harvested on day 3 after transfection) from transfection control and cells transfected with VB2060, TECH011-IV002 and TECH011-IV015, respectively, were run on an SDS-PAGE. Prior sample preparation, supernatant samples from cells transfected with TECH011-IV002 and TECH011-IV015 were diluted 1:10 in expression medium. The proteins were transferred to a PVDF-membrane. The membrane was immunoblotted using the primary antibody: goat a-human MIP-1α (AF270), and the secondary antibody: donkey a-goat, Dylight 550 (SA5-10087). Chemidoc channels Dylight 550 and 650 (for protein standard).

FIG. 27: Quantified Western Blot signal of proteins expressed from DNA plasmids

Shows quantification of the band intensities from the Western Blot shown in FIG. 26. Signal intensity was adjusted for the dilution of TECH011-IV002 and TECH011-IV015.

FIG. 28: Humoral immune response in mice

Shows the humoral immune response induced in mice administered with DNA plasmids VB2060 and TECH011-IV002 by way of measuring total IgG antibodies binding the RBD protein compared to the negative control VB1026. Mean±SEM are shown, 6 mice per group, two technical replicates shown.

FIG. 29: Expression and secretion levels of proteins encoded by DNA plasmids

Shows the expression and secretion of proteins encoded by DNA plasmids MC38-I2-WT, MC38-I2-D27A, MC38-I2-E67A and MC38-I2-D27A-E67A detected by ELISA in the supernatants of Expi293F cells transfected with said DNA plasmids. Supernatants of cells transfected with MC38-I2-WT, MC38-I2-D27A, MC38-I2-E67A and MC38-I2-D27A-E67A were harvested on day 3 after transfection and diluted, and ELISA was performed using a mouse a-human IgG CH3 domain capture Ab (MCA878G, Bio-Rad) and an a-human MIP-1α biotinylated detection Ab (BAF270, R&D systems). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 30: Expression and secretion levels of proteins encoded by DNA plasmids Shows the expression and secretion of proteins encoded by DNA plasmids MC38-I2-WT, MC38-I2-D27A, MC38-I2-E67A and MC38-I2-D27A-E67A detected by ELISA in the supernatants of Expi293F cells transfected with said DNA plasmids. Supernatants of cells transfected with MC38-I2-WT, MC38-I2-D27A, MC38-I2-E67A and MC38-I2-D27A-E67A were harvested on day 1 or day 3 after transfection and diluted. ELISA was performed using a mouse a-human IgG CH3 domain capture Ab (MCA878G, Bio-Rad) and an a-human MIP-1α biotinylated detection Ab (BAF270, R&D systems). Transfection ctrl. (=cells only treated with transfection agent ExpiFectamine™ 293 reagent which serve as a negative control). Protein concentration (in ng/mL) in the cell supernatant was interpolated from a standard curve of purified protein.

FIG. 31: CCR5 receptor activation by proteins encoded by DNA plasmids

Shows the activation of hCCR5 (CCR5-bla U2OS reporter cell line) by proteins encoded by DNA plasmids MC38-I2-WT, MC38-I2-D27A, MC38-I2-E67A and MC38-I2-D27A-E67A using supernatants of Expi293F cells transfected with said DNA plasmids. The fluorescence ratio at 460 and 530 nm detected upon beta-lactamase substrate addition and excitation at 409 nm, called the response ratio, provides a measure of the activation of hCCR5. 3 nM recombinant CCL3L1 (rCCL3L1) was used as a positive control. Transfection ctrl. (=cells only treated with transfection agent ExpiFectamine™ 293 reagent which serve as a negative control).

FIG. 32: Western Blot of proteins expressed from DNA plasmids

Western blot shows expression and secretion of intact proteins expressed from MC38-I 2-WT, MC38-I2-D27A, MC38-I2-E67A and MC38-I2-D27A-E67A by detecting the targeting unit in said proteins. Reduced supernatant samples (harvested on day 3 after transfection) from cells transfected with MC38-I2-WT, MC38-I2-D27A, MC38-I2-E67A and MC38-I2-D27A-E67A, respectively, were run on an SDS-PAGE, and the proteins were transferred to a PVDF-membrane. The membrane was immunoblotted using primary antibody: goat a-human MIP-1α (AF270). Secondary antibody: donkey a-goat, Alexa Fluor™ Plus 800 (A32930). Chemidoc channels Dylight 800 and 650 (for protein standard).

FIG. 33: Expression and secretion levels of proteins encoded by DNA plasmids

Shows the yields of purified proteins encoded by DNA plasmids VB1026, VB1026-D27A, VB1026-E67A and VB1026-D27A-E67A-P8A isolated from the supernatants of Expi293F cells transfected with said DNA plasmids on day 7 after transfection. Protein yield was assessed by UV absorption at 280 nm.

FIG. 34: Oligomerization state of proteins encoded by DNA plasmids

Shows the size exclusion chromatograms (SEC) of purified proteins encoded by DNA plasmids VB1026, VB1026-D27A, VB1026-E67A and VB1026-D27A-E67A-P8A isolated from the supernatants of Expi293F cells transfected with said DNA plasmids on day 7 after transfection.

FIG. 35: Oligomerization state of proteins encoded by DNA plasmids

Shows the size exclusion chromatograms of purified proteins encoded by DNA plasmids VB1026, VB1026-D27A, VB1026-E67A, VB1026-D27A-E67A and VB1026-D27A-E67A-P8A isolated from the supernatants of Expi293F cells transfected with said DNA plasmids on day 7 after transfection. Oligomer=all peaks with higher molecular weight than the monomer, as shown by dotted line.

FIG. 36: Quantification of oligomerization state of proteins encoded by DNA plasmids

Shows the quantification of percent monomer versus oligomer (as sum of all higher molecular weight peaks, see FIG. 35) determined by integrating the area under the curve of size exclusion chromatograms of purified proteins encoded by DNA plasmids VB1026, VB1026-D27A, VB1026-E67A, VB1026-D27A-E67A and VB1026-D27A-E67A-P8A isolated from the supernatants of Expi293F cells transfected with said DNA plasmids on day 7 after transfection. SEC was run in duplicates.

FIG. 37: Expression and secretion levels of proteins encoded by DNA plasmids

Shows the yields of purified proteins encoded by DNA plasmids mCherry-WT and mCherry-D27A-E67A-P8A isolated from the supernatants of Expi293F cells transfected with said DNA plasmids on day 7 after transfection. Protein yield was assessed by UV absorption at 280 nm. Graph shows individual biological replicates and mean±SD.

FIG. 38: Oligomerization state of proteins encoded by DNA plasmids

Shows the size exclusion chromatograms of purified proteins encoded by mCherry-WT (38A) and mCherry-D27A-E67A-P8A (38B) isolated from the supernatants of Expi293F cells transfected with said DNA plasmids on day 7 after transfection. Oligomer=all peaks with higher molecular weight than the monomer, as shown by dotted line

FIG. 39: Expression and secretion levels of proteins encoded by DNA plasmids

Shows the expression and secretion of proteins encoded by DNA plasmids MC38-I2-WT, MC38-I2-D27A, MC38-I2-E67A and MC38-I2-D27A-E67A detected by ELISA in the supernatants of mouse myoblast cells stably transfected with said DNA plasmids. Graph shows individual biological replicates and mean±SD.

FIG. 40: Expression and secretion levels of proteins encoded by DNA plasmids

Shows the expression and secretion of proteins encoded by DNA plasmids VB1026, VB1026-D27A, VB1026-D27A-E67A and VB1026-D27A-E67A-P8A detected by ELISA in the supernatants of mouse myoblast cells stably transfected with said DNA plasmids. Graph shows individual biological replicates and mean±SD.

FIG. 41: Oligomerization state of proteins encoded by DNA plasmids

Shows the size exclusion chromatograms of purified proteins encoded by DNA plasmids VB1026, VB1026-D27A, VB1026-D27A-E67A and VB1026-D27A-E67A-P8A isolated from the supernatants of mouse myoblast cells stably transfected with said DNA plasmids. Oligomer=all peaks with higher molecular weight than the monomer.

FIG. 42: Quantification of oligomerization state of proteins encoded by DNA plasmids

Shows the quantification of percent monomer versus oligomer (as sum of all higher molecular weight peaks, see FIG. 41) determined by integrating the area under the curve of size exclusion chromatograms of purified proteins encoded by DNA plasmids VB1026, VB1026-D27A, VB1026-D27A-E67A and VB1026-D27A-E67A-P8A isolated from the supernatants of mouse myoblast cells stably transfected with said DNA plasmids.

FIG. 43: Mass photometry

Shows the results of the mass photometry, i.e. quantification of monomer (peak at about 50 kDa) versus oligomer (all higher molecular weight peaks) carried out with samples of purified proteins encoded by DNA plasmids VB1026 (FIG. 43A), VB1026-D27A (FIG. 43B), VB1026-D27A-E67A (FIG. 43C) and VB1026-D27A-E67A-P8A (FIG. 43D) isolated from the supernatants of mouse myoblast cells stably transfected with said DNA plasmids.

### DETAILED DESCRIPTION

The polynucleotide, polypeptide and the multimeric protein may herein be denoted a “construct”.

The polynucleotides/polypeptides/multimeric proteins described herein are generally immunogenic constructs. An “immunogenic construct” is one that elicits an immune response, particularly when administered to a subject in a form suitable for administration and in an amount effective to elicit the immune response, i.e. an immunologically effective amount.

A “subject” is an animal, e.g. a mouse, or a human, preferably a human. The terms “mouse”, “murine” and “m” are used interchangeably herein to denote a mouse or refer to a mouse. The terms “human” and “h” are used interchangeably herein to denote a human or refer to a human. A subject may be a patient, i.e. a human suffering from a disease who is in need of a therapeutic treatment, or it may be a subject in need of prophylactic treatment, e.g., in need of prophylactic treatment from being infected with an infectious disease, or it may be a subject suspected of suffering from a disease. The terms “subject” and “individual” are used interchangeably herein.

A “disease” is an abnormal medical condition that is typically associated with specific signs and symptoms in a subject being affected by the disease.

An “infectious disease” is a disease caused by one or more pathogens, including viruses, bacteria, fungi and parasites.

A “cancer” refers to a broad group of various diseases characterized by the uncontrolled growth of abnormal cells in the body. A “cancer” or “cancer tissue” includes a tumor, and as used herein, encompasses both solid tumors as well as tumor cells found in bodily fluids such as blood, and includes metastatic cancer. Unregulated cell division and growth results in the formation of malignant tumors that can invade neighboring tissues and can also metastasize to distant parts of the body through the lymphatic system or bloodstream. Following metastasis, the distal tumors can be said to be “derived from” a pre-metastasis tumor.

A “treatment” is a prophylactic treatment or a therapeutic treatment.

A “prophylactic treatment” is a treatment administered to a subject who does not (or not yet) display signs or symptoms of, or displays only early signs or symptoms of, a disease, such that treatment is administered for the purpose of preventing or decreasing the risk of developing the disease and/or symptoms associated with the disease. A prophylactic treatment functions as a preventative treatment against a disease, or as a treatment that inhibits or reduces further development or enhancement of the disease and/or its associated symptoms. The terms prophylactic treatment, prophylaxis and prevention are used interchangeably herein.

A “therapeutic treatment” is a treatment administered to a subject who displays symptoms or signs of a disease, in which treatment is administered to the subject for the purpose of diminishing or eliminating those signs or symptoms or for the purpose of delaying or stopping disease progression.

A “T cell epitope” as used herein refers to a discrete, single T cell epitope or a part or region of an antigen containing multiple T cell epitopes, e.g. multiple minimal T cell epitopes, such as a hotspot. Consequently, a hotspot as used herein refers to part of an antigen containing multiple T cell epitopes, e.g., multiple minimal T cell epitopes.

A “nucleotide sequence” is a sequence consisting of nucleotides. The terms “nucleotide sequence” and “nucleic acid sequence” are used interchangeably herein.

A “mutation” or “mutated” as used herein is or refers to an alteration in a wild type nucleic acid sequence or amino acid sequence.

A “vector” as used herein is or refers to an expression vector, i.e., a vehicle used to carry foreign nucleotide sequences into another cell, where it can be expressed. Vectors typically contain an origin of replication and both a promoter and a cloning site into which a polynucleotide can be operatively linked.

The terms “multiple”, “several”, “a plurality” and “more” (in the sense of more than one) are used interchangeably herein.

The section headings used herein are for organizational purposes only and are not to be construed as limiting the subject matter described.

### Constructs

The polypeptides described herein comprise a mutated human CCL3 (hCCL3) or mutated human CCL3L1 (hCCL3L1) targeting unit, whose amino acid sequence comprises one or more mutations, compared to the amino acid sequence of the respective wild type, wherein said one or more mutations reduce or prevent the formation of targeting unit oligomers. Such targeting units target antigen-presenting cells (APCs). The polypeptides further comprise an antigenic unit comprising one or more antigens or parts thereof, e.g. one or more disease-relevant antigens or parts thereof, e.g. epitopes and, in some embodiments, comprise a multimerization unit, such as a dimerization unit.

Once administered to a subject, the construct elicits an immune response against the antigens or parts thereof comprised in the antigenic unit, resulting in the activation of the subject's immune system. If a polynucleotide (e.g. a vector) comprising a nucleotide sequence encoding the polypeptide is administered to a subject (in a form suitable for administration and in an amount effective to elicit the immune response), the polypeptide is expressed and elicits an immune response against the antigens or parts thereof comprised in the antigenic unit. If the polypeptide comprises a multimerization unit, it forms a multimeric protein, e.g. a dimeric protein, if the multimerization unit is a dimerization unit is a dimerization unit.

Structures like the polynucleotides, polypeptides and multimeric/dimeric proteins are known in the art (e.g. disclosed in WO 2004/076489A1, WO 2011/161244A1, WO 2017/118695A1 and WO 2022/013277A1, the disclosures of all are included herein by reference) and the skilled person can select a multimerization unit and an antigenic unit according to the envisaged use.

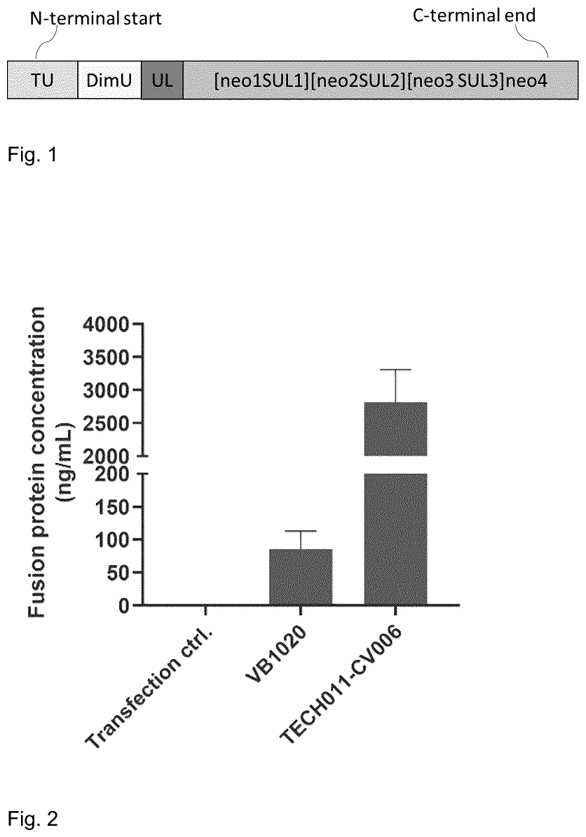

The polypeptide has an N-terminal start and a C-terminal end (illustrated in FIG. 1). The elements and units of the polypeptide—mutated hCCL3 or hCCL3L1 targeting unit as described herein (TU), multimerization unit (in FIG. 1, a multimerization unit is present in the form of a dimerization unit, (DimU)), and antigenic unit—are arranged in the polypeptide such that the antigenic unit is located at the C-terminal end of the polypeptide. A unit linker (UL) may be present and connects the multimerization unit and the antigenic unit. In the absence of a multimerization unit, the unit linker, if present, connects the targeting unit and the antigenic unit.

The antigenic unit in FIG. 1 comprises 4 neoepitopes (neo1, neo2, neo3, neo4), which are separated by linkers (SUL1, SUL2, SUL3). An alternative way to describe the arrangement of the neoepitopes neo1-neo4 is that these neoepitopes are arranged in 3 antigenic subunits, each comprising a neoepitope and a subunit linker (SUL1, SUL2, SUL3), and a terminal neoepitope (neo4), which is closest to the C-terminal end of the polypeptide. The subunits are indicated in the Figure by square brackets. Thus, an antigenic unit comprising n neoepitopes comprises n−1 subunits, each subunit comprising a neoepitope and a subunit linker. The order and orientation of the above-described units and elements of the polypeptide is the same in the multimeric protein and in the nucleic acid sequence encoding the polypeptide. A pharmaceutical composition comprising the polypeptide as shown in FIG. 1 (or the polynucleotide encoding such polypeptide) may be used as an anticancer vaccine, e.g. personalized anticancer vaccine, as described herein.

In the following, the various units and elements of polypeptide will be discussed in detail. They are present in the polynucleotide as nucleic acid sequences encoding the units/elements while they are present in the polypeptide or multimeric protein as amino acids sequences. For the ease of reading, in the following, the units/elements are mainly explained in relation to the polypeptide/multimeric protein, i.e. on the basis of their amino acid sequences.

### Targeting Unit

The polypeptide comprises a mutated human CCL3 or mutated human CCL3L1 targeting unit, wherein the amino acid sequence of the targeting unit comprises one or more mutations, compared to the amino acid sequence of the hCCL3 or hCCL3L1 wild type, which reduce or prevent the formation of targeting unit oligomers.

In some embodiments, the one or more mutations reduce or prevent the formation of targeting unit oligomers in solution. In some other embodiments, the one or more mutations reduce or prevent the formation of targeting unit oligomers in solution under relevant physiological conditions.

The term “physiological conditions” in this context refers to conditions that occur in nature in the internal milieu of the subject, e.g. in the human body. Relevant physiological conditions include temperature, pH, ion strength and concentration of hCCL3 or hCCL3L1.

Whether a mutation reduces and prevents the formation of targeting unit oligomers can be determined by comparing the state of oligomerization of the mutated hCCL3 or hCCL3L1 to that of the wild type hCCL3 or hCCL3L1, such as comparing the state of oligomerization in solution under physiological relevant conditions, by methods known in the art. Such methods include mass photometry to determine the mass of molecules in solution in their native state and determining the oligomerization state of such molecules and size exclusion chromatography (SEC) for determining the size. SEC eluents can be further characterized by techniques which are typically used to characterize biological materials/polymers, including dynamic light scattering (DLS) low/right or multiple angle light scattering (LALS, RALS, MALS) or small angle X-ray scattering (SAXS).

Alternatively, whether a mutation reduces and prevents the formation of targeting unit oligomers can be determined by comparing the state of oligomerization of a construct comprising a mutated hCCL3 or mutated hCCL3L1 targeting unit to a construct comprising a wild type hCCL3 or hCCL3L1 targeting unit, such as comparing the state of oligomerization in solution under physiological relevant conditions, by methods known in the art, e.g. those mentioned in the aforementioned paragraph. If the construct is a polynucleotide, e.g. a polynucleotide comprised in a vector, such as a plasmid, whether a mutation reduces and prevents the formation of targeting unit oligomers can be determined by comparing the concentration of the polypeptide/protein secreted from cells transfected with a polynucleotide comprising a mutated hCCL3 or mutated hCCL3L1 targeting unit encoding such polypeptide/protein to the concentration of the polypeptide/protein secreted from cells transfected with a polynucleotide comprising a wild type hCCL3 or hCCL3L1 targeting unit. Without intending to be bound by this theory, the increased section is likely due to higher solubility of the polypeptide/protein which is due to reduced or prevented oligomerization. Also truncated constructs, e.g. constructs only comprising the targeting unit and multimerization unit but no antigenic unit, can be used to determine whether a mutation reduces and prevents the formation of targeting unit oligomers, as shown in the Examples herein.

High-molecular weight oligomers of hCCL3 are formed via intermediate dimers, dimer-dimers (tetramers) and dodecamers (see for instance M. Ren et al., EMBO J 2010(29), 3952-3966). Thus, in some embodiments, said one or more mutations reduce or prevent the formation of dimer-dimers.

The targeting unit targets APCs, including dendritic cells (DCs) and subsets thereof: it attracts APCs and delivers the polypeptide/multimeric protein to APCs, where the targeting unit binds to its cognate receptors and the polypeptide/multimeric protein is internalized. Inside the APC, the polypeptide/multimeric protein is degraded, and small fractions thereof (small peptides) are loaded onto MHC molecules and presented to CD4+ and CD8+ T cells to induce specific immune responses.

Thus, the targeting unit does not only target the antigenic unit comprised in the polypeptide/multimeric protein to APCs, but also facilitates a response-amplifying effect (adjuvant effect) by recruiting APCs to the administration site of the polynucleotide/polypeptide/multimeric protein.

In some embodiments, the wild type of human CCL3 has the amino acid sequence of SEQ ID NO: 1. Thus, in some embodiments, the targeting unit is mutated hCCL3 having an amino acid sequence which comprises one or more mutations, compared to SEQ ID NO: 1, which reduce or prevent the formation of targeting unit oligomers.

In some embodiments, the wild type of human CCL3L1 has the amino acid sequence of SEQ ID NO: 2. Thus, in some embodiments, the targeting unit is mutated hCCL3L1 having an amino acid sequence which comprises one or more mutations, compared to SEQ ID NO: 2, which reduce or prevent the formation of targeting unit oligomers. In some other embodiments, the targeting unit is mutated hCCL3L1 having an amino acid sequence which, compared to SEQ ID NO: 2, is truncated at the N-terminus by 2 amino acids (i.e. amino acids 3-70 of SEQ ID NO: 2) and comprises one or more mutations, which reduce or prevent the formation of targeting unit oligomers.

In some embodiments, the mutation is a substitution of an amino acid comprised in the wild type amino acid sequence of hCCL3 or hCCL3L1 with a different amino acid. In some embodiments, the nature of the mutation and/or the number of mutations is such that it does not negatively affect the relevant protein properties of the mutated targeting unit, including its receptor binding on APCs and its ability to effect receptor signaling after receptor binding, compared to that of the wild type targeting unit.

In some embodiments, the one or more mutations do not lead to a decrease in solubility of the constructs comprising the mutated targeting unit, compared to the solubility of the constructs comprising the wild type targeting unit. The solubility can be determined by methods in the art.

In some other embodiments, the one or more mutations do not prohibit or impede the correct folding or lead to incomplete folding of a conformational antigen comprised in the antigenic unit of a construct comprising the mutated targeting unit. The correct folding of such antigen can be determined by methods known in the art, such as conformational antibodies and ELISA.

In some embodiments, the one or more mutations do not lead to a decreased ability/lack of ability of a construct comprising the mutated targeting unit to bind to its cognate receptors on APCs, such as to CCR1, CCR4 and CCR5 (hCCL3) or to CCR1, CCR3 and CCR5 (hCCL3L1), compared to that of a construct comprising the wild type targeting unit. The receptor binding ability of (mutated) hCCL3/hCCL3L1 can be determined by methods known in the art, such as flow-based binding assay using hCCR-stably transfected cells (e.g. hCCR5-stably transfected cells) to detect binding of mutated/wild type targeting unit (e.g. mutated or wild type hCCL3L1).

In some other embodiments, the one or more mutations do not lead to a decreased ability/lack of ability of a construct comprising the mutated targeting unit to elicit targeting unit-mediated responses after receptor binding (also called receptor activation or receptor signaling), compared to that of a construct comprising the wild type targeting unit. The receptor signaling ability can be determined by methods known in the art, such as the receptor signaling assay (hCCR5 reporter assay) described in the “Example” section herein.

Mutations of hCCL3 which reduce or prevent the formation of hCCL3 oligomers have been described in e.g. M. Ren et al., EMBO J 2010(29), 3952-3966, wherein hCCL3 is denoted hMIP-1α and in WO 93/13206A1, the disclosure of which is incorporated herein by reference, wherein hCCL3 is denoted LD78.

The inventors of WO 93/13206A1 stated in a later publication that while many of the mutations disclosed in WO 93/13206A1 lead to less aggregation, only a few of these mutated hCCL3 proteins retained full CCR1 receptor binding and activation activity (L. Czaplewski et al., J Biol Chem 274(23), 1999, 16077-16084, hCCL3 in this reference is denoted hMIP-1α).

In some embodiments, the mutation is designed to disrupt the salt bridge formed by amino acid residues D27 and R46 in crystalized polymers formed by the interaction of hCCL3 or hCCL3L1 wild type monomers. Thus, D27 of the wild type amino acid sequence of hCCL3 or hCCL3L1, such as of hCCL3 or hCCL3L1 having the amino acid sequence of SEQ ID NOs 1 or 2, is substituted with an amino acid that prevents the formation of the salt bridge with R46. In some embodiments, such an amino acid is a small, non-polar amino acid, such as an amino acid selected from the group consisting of G, A, V, L and I. In some embodiments, such an amino acid is selected from the group consisting of A, S and Q. In some embodiments, such an amino acid is alanine, i.e. the amino acid sequence of the targeting unit comprises a D27A mutation, compared to the amino acid sequence of the wild type.

In some embodiments, the mutation is designed to disrupt the salt bridge formed by amino acid residues E67 and R48 in crystalized polymers formed by the interaction of hCCL3 or hCCL3L1 wild type monomers. Thus, E67 of the wild type amino acid sequence of hCCL3 or hCCL3L1, such as of hCCL3 or hCCL3L1 having the amino acid sequence of SEQ ID NOs 1 or 2, is substituted with an amino acid that prevents the formation of the salt bridge with R48. In some embodiments, such an amino acid is a small, non-polar amino acid, such as an amino acid selected from the group consisting of G, A, V, L and I. In some embodiments, such an amino acid is selected from the group consisting of A, S and Q. In some embodiments, such an amino acid is alanine, i.e. the amino acid sequence of the targeting unit comprises an E67A mutation, compared to the amino acid sequence of the wild type.

In some embodiments, the mutation is designed to disrupt the hydrogen bond between amino acid residues D6 and S33 in crystalized polymers formed by the interaction of hCCL3 or hCCL3L1 wild type monomers. Thus, D6 of the wild type amino acid sequence of hCCL3 or hCCL3L1, such as of hCCL3 or hCCL3L1 having the amino acid sequence of SEQ ID NOs 1 or 2, is substituted with an amino acid that prevents the formation of the hydrogen bond with S33. In some embodiments, such an amino acid is a non-polar amino acid. In some other embodiments, such an amino acid is a small, non-polar amino acid. In some embodiments, such an amino acid is selected from the group of G, A, V, L or I. In some embodiments, such an amino acid is alanine, i.e. the amino acid sequence of the targeting unit comprises a D6A mutation, compared to the amino acid sequence of the wild type.

In some embodiments, the mutation is designed to disrupt the hydrophobic interactions of amino acid residue F24 in crystalized polymers formed by the interaction of hCCL3 or hCCL3L1 wild type monomers. Thus, F24 of the wild type amino acid sequence of hCCL3 or hCCL3L1, such as of hCCL3 or hCCL3L1 having the amino acid sequence of SEQ ID NOs 1 or 2, is substituted with an amino acid that prevents such hydrophobic interactions. In some embodiments, such an amino acid is a non-polar amino acid. In some other embodiments, such an amino acid is a small, non-polar amino acid. In some embodiments, such an amino acid is selected from the group of G, A, V, L or I. In some embodiments, such an amino acid is alanine, i.e. the amino acid sequence of the targeting unit comprises a F24A mutation, compared to the amino acid sequence of the wild type.

In some embodiments, the mutation is designed to disrupt the hydrophobic interactions of amino acid residue F29 in crystalized polymers formed by the interaction of hCCL3 or hCCL3L1 wild type monomers. Thus, F29 of the wild type amino acid sequence of hCCL3 or hCCL3L1, such as of hCCL3 or hCCL3L1 having the amino acid sequence of SEQ ID NOs 1 or 2, is substituted with an amino acid that prevents such hydrophobic interactions. In some embodiments, such an amino acid is a non-polar amino acid. In some other embodiments, such an amino acid is a small, non-polar amino acid. In some embodiments, such an amino acid is selected from the group of G, A, V, L or I. In some embodiments, such an amino acid is alanine, i.e. the amino acid sequence of the targeting unit comprises a F29A mutation, compared to the amino acid sequence of the wild type.

In some embodiments, the mutation is designed to disrupt the hydrophobic interactions of amino acid residue Y28 in crystalized polymers formed by the interaction of hCCL3 or hCCL3L1 wild type monomers. Thus, Y28 of the wild type amino acid sequence of hCCL3 or hCCL3L1, such as of hCCL3 or hCCL3L1 having the amino acid sequence of SEQ ID NOs 1 or 2, is substituted with an amino acid that prevents such hydrophobic interactions. In some embodiments, such an amino acid is a non-polar amino acid. In some other embodiments, such an amino acid is a small non-polar amino acid. In some embodiments, such an amino acid is selected from the group of G, A, V, L or I. In some embodiments, such an amino acid is alanine, i.e. the amino acid sequence of the targeting unit comprises a Y28A mutation, compared to the amino acid sequence of the wild type.

In some embodiments, the mutation substitutes the amino acid residue P8 with a non-polar amino acid. Thus, P8 of the wild type amino acid sequence of hCCL3 or hCCL3L1, such as of hCCL3 or hCCL3L1 having the amino acid sequence of SEQ ID NOs 1 or 2, is substituted with a non-polar amino acid. In some other embodiments, such an amino acid is a small non-polar amino acid. In some embodiments, such an amino acid is selected from the group of G, A, V, L or I. In some embodiments, such an amino acid is alanine, i.e. the amino acid sequence of the targeting unit comprises a P8A mutation, compared to the amino acid sequence of the wild type.

In some embodiments, the targeting unit comprises one mutation. In some embodiments, the targeting unit comprises a mutation which is designed to disrupt the salt bridge formed by amino acid residues D27 and R46 in crystalized polymers formed by the interaction of hCCL3 or hCCL3L1 wild type monomers. In some embodiments, the targeting unit comprises a D27A mutation. In some other embodiments, the targeting unit comprises a mutation which is designed to disrupt the salt bridge formed by amino acid residues E67 and R48 in crystalized polymers formed by the interaction of hCCL3 or hCCL3L1 wild type monomers. In some embodiments, the targeting unit comprises an E67A mutation.

In some embodiments, the targeting unit comprises the amino acid sequence of SEQ ID NO: 1 which comprises a D27A mutation. In some embodiments, the targeting unit comprises the amino acid sequence of SEQ ID NO: 3. In some embodiments, the targeting unit consists of the amino acid sequence of SEQ ID NO: 3.

In some embodiments, the targeting unit comprises the amino acid sequence of SEQ ID NO: 1 which comprises an E67A mutation. In some embodiments, the targeting unit comprises the amino acid sequence of SEQ ID NO: 4. In some embodiments, the targeting unit consists of the amino acid sequence of SEQ ID NO: 4.

In some embodiments, the targeting unit comprises the amino acid sequence of SEQ ID NO: 2 which comprises a D27A mutation. In some embodiments, the targeting unit comprises the amino acid sequence of SEQ ID NO: 5. In some embodiments, the targeting unit consists of the amino acid sequence of SEQ ID NO: 5. In some embodiments, the targeting unit comprises the amino acid sequence 3-70 of SEQ ID NO: 2 which comprises a D27A mutation. In some embodiments, the targeting unit comprises the amino acid sequence 3-70 of SEQ ID NO: 5. In some embodiments, the targeting unit consists of the amino acid sequence 3-70 of SEQ ID NO: 5.

In some embodiments, the targeting unit comprises the amino acid sequence of SEQ ID NO: 2 which comprises an E67A mutation. In some embodiments, the targeting unit comprises the amino acid sequence of SEQ ID NO: 6. In some embodiments, the targeting unit consists of the amino acid sequence of SEQ ID NO: 6. In some embodiments, the targeting unit comprises the amino acid sequence 3-70 of SEQ ID NO: 2 which comprises a E67A mutation. In some embodiments, the targeting unit comprises the amino acid sequence 3-70 of SEQ ID NO: 6. In some embodiments, the targeting unit consists of the amino acid sequence 3-70 of SEQ ID NO: 6.

In some other embodiments, the targeting unit comprises more than one mutation, i.e. multiple mutations, such as 2 or 3 or 4 or 5 mutations.

In some embodiments, the amino acid sequence of the targeting unit comprises a D27A and an E67A mutation, compared to the amino acid sequence of the wild type. In some other embodiments, the amino acid sequence of the targeting unit comprises a D27A and a P8A mutation, compared to the amino acid sequence of the wild type. In yet some other embodiments, the amino acid sequence of the targeting unit comprises an E67A and a P8A mutation, compared to the amino acid sequence of the wild type. In yet some other embodiments, the amino acid sequence of the targeting unit comprises a D27A mutation, an E67A and a P8A mutation, compared to the amino acid sequence of the wild type.

In some embodiments, the targeting unit comprises a first mutation which is designed to disrupt the salt bridge formed by amino acid residues D27 and R46 and a second mutation which is designed to disrupt the salt bridge formed by amino acid residues E67 and R48 in crystalized polymers formed by the interaction of hCCL3 or hCCL3L1 monomers.

In some embodiments, the targeting unit comprises the amino acid sequence of SEQ ID NO: 1 which comprises a D27A and an E67A mutation. In some embodiments, the targeting unit comprises the amino acid sequence of SEQ ID NO: 7. In some embodiments, the targeting unit consists of the amino acid sequence of SEQ ID NO: 7.

In some embodiments, the targeting unit comprises the amino acid sequence of SEQ ID NO: 2 which comprises a D27A and an E67A mutation. In some embodiments, the targeting unit comprises the amino acid sequence of SEQ ID NO: 8. In some embodiments, the targeting unit consists of the amino acid sequence of SEQ ID NO: 8. In some embodiments, the targeting unit comprises the amino acid sequence 3-70 of SEQ ID NO: 2 which comprises a D27A and an E67A mutation. In some embodiments, the targeting unit comprises the amino acid sequence 3-70 of SEQ ID NO: 8. In some embodiments, the targeting unit consists of the amino acid sequence 3-70 of SEQ ID NO: 8.

In some embodiments, the targeting unit comprises the amino acid sequence of SEQ ID NO: 1 which comprises a D27A, an E67A and a P8A mutation. In some embodiments, the targeting unit comprises the amino acid sequence of SEQ ID NO: 140. In some embodiments, the targeting unit consists of the amino acid sequence of SEQ ID NO: 140.

In some embodiments, the targeting unit comprises the amino acid sequence of SEQ ID NO: 2 which comprises a D27A, an E67A and a P8A mutation. In some embodiments, the targeting unit comprises the amino acid sequence of SEQ ID NO: 141. In some embodiments, the targeting unit consists of the amino acid sequence of SEQ ID NO: 141. In some embodiments, the targeting unit comprises the amino acid sequence 3-70 of SEQ ID NO: 2 which comprises a D27A, an E67A and a P8A mutation. In some embodiments, the targeting unit comprises the amino acid sequence 3-70 of SEQ ID NO: 141. In some embodiments, the targeting unit consists of the amino acid sequence 3-70 of SEQ ID NO: 141.

In some embodiments, the targeting unit comprises or consists of a nucleic acid sequence selected from the list consisting of SEQ ID NO: 139, SEQ ID NO: 188, SEQ ID NO: 189 and SEQ ID NO: 190.

In some embodiments, the targeting unit comprises the amino acid sequence of SEQ ID NO: 5. In some embodiments, the targeting unit consists of the amino acid sequence of SEQ ID NO: 5. In some embodiments, the targeting unit comprises the amino acid sequence 3-70 of SEQ ID NO: 5. In some embodiments, the targeting unit consists of the amino acid sequence 3-70 of SEQ ID NO: 5. In some embodiments, the targeting unit comprises the nucleic acid sequence of SEQ ID NO: 139. In some embodiments, the targeting unit consists of the nucleic acid sequence of SEQ ID NO: 139.

### Multimerization Unit/Dimerization Unit

In some embodiments, the constructs disclosed herein comprise a multimerization unit, such as a dimerization unit. In some preferred embodiments, the constructs comprise a multimerization unit, such as a dimerization unit.

The term “multimerization unit” as used herein refers to a sequence of nucleotides or amino acids between the antigenic unit and the targeting unit which, in addition to connecting the antigenic unit and the targeting unit, facilitates multimerization of/joins multiple polypeptides, such as two, three, four or more polypeptides, into a multimeric protein, such as a dimeric protein, a trimeric protein or a tetrameric protein. Furthermore, the multimerization unit also provides flexibility in the multimeric protein to allow optimal binding of the targeting unit to the surface molecules on the APCs, even if they are located at variable distances. The multimerization unit may be any unit that fulfils one or more of these requirements.

Multimerization Unit that Facilitates Multimerization of/Joins More than Two Polypeptides

In some embodiments, the multimerization unit is a trimerization unit, such as a collagen-derived trimerization unit, such as a human collagen-derived trimerization domain, such as human collagen XVIII-derived trimerization domain (see for instance A. Alvarez-Cienfuegos et al., Sci Rep 6, 28643 (2016)) or human collagen XV-derived trimerization domain. Thus, in some embodiments, the multimerization unit is a trimerization unit that comprises or consists of the nucleic acid sequence of SEQ ID NO: 9, or comprises or consists of an amino acid sequence encoded by said nucleic acid sequence. In some other embodiments, the trimerization unit is the C-terminal domain of T4 fibritin. Thus, in some embodiments, the multimerization unit is a trimerization unit that comprises or consists of the amino acid sequence of SEQ ID NO: 10. In some embodiments, the trimerization unit further comprises a hinge region as described below.

In some other embodiments, the multimerization unit is a tetramerization unit, such as a domain derived from p53, optionally further comprising a hinge region as described below. Thus, in some embodiments, the multimerization unit is a tetramerization unit that comprises or consists of the nucleic acid sequence of SEQ ID NO: 11, or comprises or consists of an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit, which, in addition to connecting the antigenic unit and the targeting unit, facilitates dimerization of/joins two monomeric polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils these requirements.

Accordingly, in some embodiments the polypeptide comprises a dimerization unit comprising a hinge region. In some other embodiments, the dimerization unit comprises a hinge region and another domain that facilitates dimerization. In yet some other embodiments, the dimerization unit comprises a hinge region, a dimerization unit linker and another domain that facilitates dimerization, wherein the dimerization unit linker connects the hinge region and the other domain that facilitates dimerization. In some embodiments, the dimerization unit linker is a glycine-serine rich linker, preferably GGGSSGGGSG (SEQ ID NO: 12), i.e. the dimerization unit comprises a glycine-serine rich dimerization unit linker and preferably comprises the dimerization unit linker GGGSSGGGSG.

The term “hinge region” refers to an amino acid sequence comprised in the dimerization unit that contributes to joining two of the polypeptides, i.e. facilitates the formation of a dimeric protein. Moreover, the hinge region functions as a flexible spacer, allowing the two targeting units of the dimeric protein to bind simultaneously to two surface molecules on APCs, even if they are located at variable distances. In the context of a multimerization unit that facilitates multimerization of/joins more than two polypeptides, the term “hinge region” refers to an amino acid sequence comprised in such multimerization unit that contributes to joining more than two polypeptides, e.g. three or four polypeptides and/or functioning as a flexible spacer, allowing the multiple targeting units of the multimeric protein to bind simultaneously to multiple surface molecules on APCs, even if they are located at variable distances.

The hinge region may be Ig derived, such as derived from IgG, e.g. IgG1 or IgG2 or IgG3, such as derived from human Ig, such as derived from human IgG, e.g. hIgG1 or hIgG2 or hIgG3. In some embodiments, the hinge region is derived from IgM, such as derived from human IgM. In some embodiments, the hinge region comprises or consists of the nucleotide sequence with SEQ ID NO: 13 or comprises or consists of an amino acid sequence encoded by said nucleic acid sequence. The hinge region may contribute to the dimerization through the formation of covalent bond(s), e.g. disulfide bridge(s) between cysteines. Thus, in some embodiments, the hinge region has the ability to form one or more covalent bonds. Preferably, the covalent bond is a disulfide bridge.

In some embodiments, the dimerization unit comprises or consists of a hinge exon h1 and hinge exon h4 (human hinge region 1 and human hinge region 4), preferably hinge exon h1 and hinge exon h4 from IgG3, more preferably having an amino acid sequence of at least 80% sequence identity to the amino acid sequence of SEQ ID NO: 14.

In a preferred embodiment, the dimerization unit comprises or consists of a hinge exon h1 and hinge exon h4 with an amino acid sequence of at least 85% sequence identity to the amino acid sequence of SEQ ID NO: 14, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a hinge exon h1 and hinge exon h4 with the amino acid sequence of SEQ ID NO: 14.

In a preferred embodiment, the dimerization unit comprises or consists of the amino acid sequence of SEQ ID NO: 14, except that at the most four amino acids have been substituted, deleted or inserted, such as at the most three amino acids, such as at the most two amino acids or such as at the most one amino acid.

In a preferred embodiment, the dimerization unit comprises or consists of a hinge exon h1 and hinge exon h4 with a nucleic acid sequence having at least 80% sequence identity to the nucleic acid sequence of SEQ ID NO: 15.

In a further preferred embodiment, the dimerization unit comprises or consists of a hinge exon h1 and hinge exon h4 with a nucleic acid sequence having at least 85% sequence identity to the nucleic acid sequence of SEQ ID NO: 15, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of a hinge exon h1 and hinge exon h4 with the nucleic acid sequence of SEQ ID NO: 15.

In some other embodiments, the dimerization unit comprises another domain that facilitates dimerization. In some embodiments, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG, such as from human IgG3. More preferably, the other domain that facilitates dimerization is a carboxyterminal C domain derived from IgG3, such as from human IgG3.

In some embodiments, the dimerization unit comprises or consists of a carboxyterminal C domain derived from IgG3 with an amino acid sequence having at least 80% sequence identity to the amino acid sequence of SEQ ID NO: 16.

In a preferred embodiment, the dimerization unit comprises or consists of a carboxyterminal C domain derived from IgG3 with an amino acid sequence having at least 85% sequence identity to the amino acid sequence of SEQ ID NO: 16, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a carboxyterminal C domain derived from IgG3 with the amino acid sequence of SEQ ID NO: 16.

In a preferred embodiment, the dimerization unit comprises or consists of the amino acid sequence of SEQ ID NO: 16, except that at the most 16 amino acids have been substituted, deleted or inserted, such as at the most 15, 14, 13, 12, 11, 10, 9, 8, 7, 6, 5, 4, 3, 2, or 1 amino acid.

In one preferred embodiment, the dimerization unit comprises or consists of a carboxyterminal C domain derived from IgG3 with the nucleic acid sequence having at least 80% sequence identity to the nucleic acid sequence of SEQ ID NO: 17.

In a further preferred embodiment, the dimerization unit comprises or consists of a carboxyterminal C domain derived from IgG3 with a nucleic acid sequence having at least 85% sequence identity to the nucleic acid sequence of SEQ ID NO: 17, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of a carboxyterminal C domain derived from IgG3 with the nucleic acid sequence of SEQ ID NO: 17.

The immunoglobulin domain contributes to dimerization through non-covalent interactions, e.g. hydrophobic interactions. Thus, in some embodiments, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3. In another preferred embodiment, the dimerization unit consists of a polypeptide consisting of hinge exon h1, hinge exon h4, a dimerization unit linker and a CH3 domain of human IgG3.

In some embodiments, the dimerization unit comprises an amino acid sequence having at least 80% sequence identity to the amino acid sequence SEQ ID NO: 18.

In a preferred embodiment, the dimerization unit comprises an amino acid sequence having at least 85% sequence identity to the amino acid sequence SEQ ID NO: 18, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In an even more preferred embodiment, the dimerization unit comprises the amino acid sequence of SEQ ID NO: 18.

In a more preferred embodiment the dimerization unit consists of an amino acid sequence having at least 80% sequence identity to the amino acid sequence of SEQ ID NO: 18, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence of SEQ ID NO: 18.

In a preferred embodiment, the dimerization unit comprises or consists of the amino acid sequence of SEQ ID NO: 18, except that at the most 28 amino acids have been substituted, deleted or inserted, such as at the most 25, 20, 15, 14, 13, 12, 11, 10, 9, 8, 7, 6, 5, 4, 3, 2 or 1 amino acids.

In one preferred embodiment, the dimerization unit comprises or consists of a nucleic acid sequence having at least 80% sequence identity to the nucleic acid sequence of SEQ ID NO: 19.

In a further preferred embodiment, the dimerization unit comprises or consists of a nucleic acid sequence having at least 85% sequence identity to the nucleic acid sequence of SEQ ID NO: 19, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 19.

### Antigenic Unit

Generally, the antigenic unit comprised in the polypeptide/multimeric protein can comprise any type of antigen(s) or parts thereof, e.g. antigens or parts thereof which are disease-relevant. Examples include one or more cancer antigens or parts thereof or one or more antigens or parts thereof relevant for an infectious disease, i.e. a disease caused by a pathogen, including viruses, bacteria, fungi and parasites.

“Disease-relevant antigen(s)” or “antigen(s) which is/are relevant for a disease” is used herein to describe that the antigen(s) or parts thereof included in the antigenic unit play a role in/have a relevance for a certain disease for which the construct as disclosed herein comprising such antigenic unit is designed to be used. As an example, the antigenic unit comprises one or more cancer antigens or parts thereof and a construct comprising such antigenic unit is designed for use in the treatment of cancer. In another example, the antigenic unit comprises one or more antigens or parts thereof derived from a pathogen and a construct comprising such antigenic unit is designed for use in the treatment of an infectious disease caused by such pathogen or wherein such pathogen is involved.

A “part” refers to a part/fragment of an antigen, i.e. part/fragment of the amino acid sequence of an antigen, or the nucleotide sequence encoding same, e.g. an epitope.

### Antigenic Unit of Individualized Constructs for Use in Personalized Anticancer Treatment

In some embodiments, the polypeptide comprises an antigenic unit, which is designed specifically and only for the patient who is to be treated with the polypeptide/multimeric protein or the polynucleotide comprising a nucleotide sequence encoding the polypeptide. Thus, the antigenic unit of such a polypeptide comprises one or more patient-specific cancer antigens or parts thereof, such antigens include neoantigens or patient-present shared cancer antigens.

“Patient-present shared cancer antigen” is used herein to describe a shared cancer antigen or shared tumor antigen, such as a shared tumor-associated antigen or shared tumor-specific antigen that has been identified to be present in the patient's tumor cells.

“Patient-present shared cancer epitope” is a part of a patient-present shared cancer antigen and used herein to describe an amino acid sequence, or a nucleic acid sequence encoding same, comprised in a patient-present shared cancer antigen, which is known to be immunogenic or which has been predicted to be immunogenic.

“Neoantigen” is used herein to describe a cancer antigen or tumor antigen found in a patient's tumor cells that comprises one or more mutations compared to the same patient's normal cells (i.e. healthy, non-cancerous cells).

“Neoepitope” is a part of a neoantigen and used herein to describe an amino acid sequence, or a nucleic acid sequence encoding same, comprised in a neoantigen which is predicted to be immunogenic. In some embodiments, the term “neoepitope” is used herein to describe an amino acid sequence, or a nucleic acid sequence encoding same, comprised in a patient-present shared tumor-specific antigen, which is known to be immunogenic or which has been predicted to be immunogenic.

Thus, in one aspect, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more patient-specific cancer antigens or parts thereof, such as one or more patient-present shared cancer antigens or parts thereof and/or one or more neoantigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in one aspect, the disclosure relates to a construct, the construct being:
(i) a polypeptide comprising a targeting unit selected from the group consisting of mutated human CCL3 and mutated human CCL3L1 and an antigenic unit comprising one or more patient-specific cancer antigens or parts thereof, such as one or more patient-present shared cancer antigens or parts thereof and/or one or more neoantigens or parts thereof; wherein targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more patient-specific cancer antigens or parts thereof, such as one or more patient-present shared cancer antigens or parts thereof and/or one or more neoantigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more patient-specific cancer antigens or parts thereof, such as one or more patient-present shared cancer antigens or parts thereof and/or one or more neoantigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i);

In some embodiments, the antigenic unit comprises one or more patient-present shared cancer antigens or parts thereof, e.g. one patient-present shared cancer antigen or one or more parts of such patient-present shared cancer antigen, e.g. one or more epitopes, or several patient-present shared cancer antigens or one or more parts of such several patient-present shared cancer antigens, e.g. one or more epitopes.

In some other embodiments, the antigenic unit comprises one or more neoantigens or parts thereof, e.g. one neoantigen or one or more parts of such neoantigen, e.g. one or more neoepitopes or several neoantigens or one or more parts of such several neoantigens, e.g. one or more neoepitopes.

In yet some other embodiments, the antigenic unit comprises any combinations of the aforementioned embodiments, i.e. any combination of one or more patient-present shared cancer antigens or parts thereof and of one or more neoantigens or parts thereof mentioned above.

### Antigenic Unit of Individualized Constructs Comprising One or More Neoantigens or Parts Thereof

Cancers develop from the patient's normal tissue by one or a few cells starting an abnormal, uncontrolled proliferation of the cells due to mutations. Although the cancer cells are mutated, most of the genome is intact and identical to the remaining cells in the patient. One approach of attacking a tumor is based on the knowledge that any tumor in any patient is unique: patient-specific mutations lead to expression of patient-specific mutated proteins, i.e. neoantigens that are unique for the particular patient. These neoantigens are not identical to any proteins in the normal cells of the patient. Therefore, such neoantigens are suitable targets for a therapeutic pharmaceutical composition comprising vector of the invention which is manufactured specifically and only for the patient in question, i.e. an individualized anticancer vaccine.

The mutation may be any mutation leading to a change in at least one amino acid. Accordingly, the mutation may be one of the following:
a non-synonymous mutation leading to a change in the amino acid
a mutation leading to a frame shift and thereby a completely different open reading frame in the direction after the mutation
a read-through mutation in which a stop codon is modified or deleted leading to a longer protein with a tumor-specific epitope
splice mutations that lead to a unique tumor-specific protein sequence
chromosomal rearrangements that give rise to a chimeric protein with a tumor-specific epitope at the junction of the two proteins. When the mutation is due to a chromosomal rearrangement, the tumor-specific epitope can arise from a change in at least one amino acid or from a combination of two in-frame coding sequences.

In some embodiments, the antigenic unit comprises one or more neoantigens or parts thereof, such as one or more parts of one neoantigen or one or more parts of several neoantigens, preferably one or more neoepitopes and more preferably several neoepitopes. Such neoepitopes may be selected for inclusion into antigenic unit according to their predicted therapeutic efficacy, see WO 2017/118695A1, the disclosures of which is incorporated herein by reference.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more neoantigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more neoantigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more neoantigens or parts thereof; or
(ii) a polypeptide encoded by the nucleotide sequence as defined in (i); or (iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more neoantigens or parts thereof, wherein the targeting unit comprises one or more mutations, compared to the wild type, which reduce or prevent the formation of targeting unit oligomers; or
(ii) a polynucleotide encoding the polypeptide as defined in (i);
(iii) a multimeric protein consisting of multiple polypeptides as defined in (i);

In some embodiments, the antigenic unit comprises one or more parts of one neoantigen or one or more parts of several neoantigens, preferably one or more neoepitopes. In some embodiments, in the antigenic unit, some of the neoepitopes are separated by linkers. In some other embodiments, all the neoepitopes are separated by linkers. If all the neoepitopes are separated by linkers, an alternative way to describe the antigenic unit is that all neoepitopes but the terminal neoepitope, i.e. the neoepitope at the C-terminal end of the polypeptide/multimeric protein, are arranged in antigenic subunits, wherein each subunit comprises a neoepitope and a subunit linker.

The neoepitope preferably has a length suitable for presentation by HLA molecules. Thus, in a preferred embodiment, the neoepitope has a length of from 7 to 30 amino acids. More preferred are neoepitopes having a length of from 7 to 10 amino acids or of from 13 to 30 amino acids, e.g. from 20 to 30 amino acids, e.g. 27 amino acids.

Preferably, the antigenic unit comprises a plurality of neoepitopes. In some embodiments, the antigenic unit comprises a plurality of different neoepitopes. In some other embodiments, the antigenic unit comprises multiple copies of the same neoepitope. In yet some other embodiments, the antigenic unit comprises several different neoepitopes and multiple copies of the same neoepitope.

Accordingly, a preferred approach is to include as many neoepitopes as possible in the antigenic unit (i.e. different and/or multiple copies of the same neoepitope) to thereby attack the cancer efficiently whilst not compromising the ability to activate T cells against the neoepitopes due to dilution of the desired T cell effect.

To design the antigenic unit, the patient's tumor exome is analyzed to identify neoantigens. Preferably, the sequences of the most immunogenic neoepitopes from one or more neoantigens are selected for inclusion into the antigenic unit.

In some embodiments, the antigenic unit comprises at least 1 neoepitope. Preferably, the antigenic unit comprises at least 3 neoepitopes, more preferably at least 5 neoepitopes, such as 7 neoepitopes. In another more preferred embodiment, the antigenic unit comprises at least 10 neoepitope. In another more preferred embodiment, the antigenic unit comprises at least 15 neoepitopes, such as at least 20 or at least 25 or at least 30 or at least 35 or at least 40 or at least 45 neoepitopes.

Antigenic units comprising one or more neoepitopes are described in detail in WO 2017/118695A1. Any of such antigenic units can be used as antigenic unit in the constructs of the invention for use in individualized anticancer therapy.

### Antigenic Unit of Individualized Constructs Comprising One or More Patient-Present Shared Cancer Antigens or Parts Thereof

Shared tumor antigens are expressed by many tumors, either across patients with the same cancer type, or across patients and cancer types. An example is the HPV16 antigen, a viral antigen that is expressed in about 50% of all patients with squamous cell carcinoma of the head and neck, but also in patients with other cancers such as cervical cancer and vulvar squamous cell carcinoma. Many of these shared tumor antigens have previously been characterized as immunogenic and/or are known, i.e. their immunogenicity has been confirmed by appropriate methods and the results have been published, e.g. in a scientific publication. Others have already been predicted to be presented on specific HLA class I or class II alleles, e.g. by algorithms known in the art and their predicted immunogenicity has been published, e.g. in a scientific publication, without having confirmed their immunogenicity by appropriate methods.

In some embodiments, the antigenic unit comprises one or more patient-present shared cancer antigens or parts thereof, e.g. patient-present shared cancer epitopes, which are known to be immunogenic, have known expression patterns and/or are known or have already been predicted to bind to specific HLA class I and class II molecules.

T cells specific to patient-present shared cancer antigens can travel to the tumor and affect the tumor microenvironment, thus increasing the likelihood that additional tumor-specific T cells are able to attack the cancer.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more one or more patient-present shared cancer antigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some in some embodiments, the disclosure relates to a construct, the construct being:
(i) a polypeptide comprising a targeting unit selected from the group consisting of mutated human CCL3 and mutated human CCL3L1 and an antigenic unit comprising one or more one or more patient-present shared cancer antigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more patient-present shared cancer antigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more one or more patient-present shared cancer antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

Some patient-present shared cancer antigens are proteins comprising an amino acid sequence that comprise one or more mutations, i.e. patient-present shared cancer epitopes which are known to be immunogenic or which have been predicted to be immunogenic. Other patient-present shared cancer antigens are proteins which do not comprise mutations, but which are overexpressed or expressed in tissues where they are normally not expressed.

In some embodiments, the patient-present shared cancer antigen is selected from the group consisting of overexpressed cellular proteins, aberrantly expressed cellular proteins, cancer testis antigens, viral antigens, differentiation antigens, mutated oncogenes, mutated tumor suppressor genes, oncofetal antigens, shared fusion antigens, shared intron retention antigens, dark matter antigens, shared antigens caused by spliceosome mutations and shared antigens caused by frameshift mutations.

In some embodiments, the patient-present shared cancer antigen is an overexpressed or aberrantly expressed human cellular protein, i.e. a cellular protein found at increased levels in tumors compared with normal healthy cells and tissues. Examples of such overexpressed or aberrantly expressed cellular proteins include tumor protein D52, Her-2/neu, hTERT (telomerase) and survivin.

In some other embodiments, the patient-present shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet some other embodiments, the patient-present shared cancer antigen is a differentiation antigen, for example tyrosinase.

In yet some other embodiments, the patient-present shared antigen is a viral antigen. Examples of viral antigens include human papilloma virus (HPV), hepatitis B virus (HBV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet some other embodiments, the patient-present shared cancer antigen is a mutated oncogene. Examples of mutated oncogenes include KRAS, CALR and TRP-2.

In yet some other embodiments, the patient-present shared cancer antigen is a mutated tumor suppressor gene. Examples include mutated p53, mutated pRB, mutated BCL2 and mutated SWI/SNF.

In yet some other embodiments, the patient-present shared cancer antigen is an oncofetal antigen, for example alpha-fetoprotein or carcinoembryonic antigen.

In yet some other embodiments, the patient-present shared antigen is a shared intron retention antigen or shared antigen caused by frameshift mutation, for example CDX2 or CALR.

In yet some other embodiments, the patient-present shared antigen is a shared antigen caused by spliceosome mutations. An example is an antigen caused by mutations like SF3B1 mut.

Further examples of shared cancer antigens include scFvs derived from a monoclonal Ig produced by myeloma or lymphoma, also called the myeloma/lymphoma M component in patients with B cell lymphoma or multiple myeloma, HIV derived sequences like e. g. gpl20 or Gag derived sequences, tyrosinase related protein (TRP)-1, melanoma antigen, prostate specific antigen and idiotypes, HPV antigens selected from the list consisting of E1, E2, E6, E7, L1 and L2, e.g. E6 and/or E7 of HPV16 and/or HPV18.

Generally, for any cancer antigen, immune tolerance to the cancer antigen has likely occurred, when a patient presents with cancer. An anticancer vaccine should specifically trigger immune response to the antigens incorporated in the vaccine. In some embodiments, the constructs of the invention are used as an anticancer vaccine. The peripheral immune tolerance to the selected antigens may be weak or strong. By incorporating such patient-present shared cancer antigens or one or more parts thereof in the antigenic unit—either alone or together with other patient-present shared cancer antigens or parts thereof and/or neoantigens or neoepitopes—a construct comprising such antigenic unit elicits an immune response which is strong and broad enough to affect the tumor microenvironment and change the patient's immune response against the tumor from a suppressive/tolerated type to a pro-inflammatory type. This may help to break tolerance to several other antigens, thus representing a considerable clinical benefit for the patient. The afore-described concept may be referred to as tipping the cancer immunity set point.

In some embodiments the antigenic unit comprises one or more patient-present shared cancer antigens or parts thereof that is a human cellular protein, preferably an overexpressed or aberrantly expressed human cellular protein or a differentiation antigen.

The patient-present shared cancer antigen can be detected in the tissue or body fluid of the patient by methods known in the art, including:
sequencing the patient's genome or exome and optionally searching, e.g. by tailor made software in whole genome/exome-seq data to e.g. identify mutated oncogenes or mutated tumor suppressor genes;
immunohistochemistry of the patient's tumor tissue, e.g. to detect the presence of mutated proteins;
RT-PCR, e.g. to detect the presence of viral antigens or known mutations in oncogenes;
ELISA using antibodies against e.g. mutated tumor proteins in serum samples;
RNA-seq of tumor tissue and comparison to healthy tissue to e.g. detect expression/over-expression of shared cancer antigens;
searching, e.g. by tailor-made software in raw RNA sequence data to identify intron retention antigens;
searching, e.g. by tailor-made software, in whole genome-seq data to identify transposable elements which are elements of dark matter antigens;
detection of short repeats in raw whole exome/RNA sequence data to e.g. identify dark matter antigens;
RNA-seq data to e.g. identify shared viral antigens; and
comparing RNA-seq of the patient's tumor samples with either patient's own healthy tissue or a cohort/database (e.g. TCGA) versus consensus transcript expression, such as GTEX/HPA gene expression data.

In some preferred embodiments, the antigenic unit comprises one or more patient-present shared cancer antigens or part(s) of such antigen(s) that are known to be immunogenic, i.e. have been studied, proposed and/or verified to be involved and of relevance for cancer and published, e.g., in the scientific literature, such as have previously been described to elicit an immune response in other patients, or have been predicted to bind to the patient's HLA class I and/or class II alleles.

In some embodiments, the antigenic unit comprises one or more patient-present shared cancer epitopes. In a preferred embodiment, such epitopes have a length suitable for presentation by the patient's HLA alleles.

In some embodiments, the antigenic unit comprises one or more patient-present shared cancer epitopes having a length suitable for specific presentation on HLA class I or HLA class II. In some embodiments, the epitope has a length of from 7 to 11 amino acids for HLA class I presentation. In some other embodiments, the epitope has a length of from 13 to 30 amino acids for HLA class II presentation.

In some embodiments, the antigenic unit comprises one or more patient-present shared cancer epitopes having a length of from 7 to 30 amino acids, e.g. from 7 to 10 amino acids (such as 7, 8, 9, or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

The antigenic unit may comprise one or more patient-present shared cancer antigens either in full-length or one or more parts thereof.

In some embodiments, the antigenic unit comprises one patient-present shared cancer antigen in full-length. In some other embodiments, the antigenic unit comprises several patient-present shared cancer antigens, each of them in full-length.

In yet some other embodiments, the antigenic unit comprises one or more parts of a patient-present shared cancer antigen, e.g. one or more patient-present shared cancer epitopes. In yet some other embodiments, the antigenic unit comprises one or more parts of several patient-present shared cancer antigens, e.g. one or more epitopes of several patient-present shared cancer antigens.

In yet some other embodiments, the antigenic unit comprises one or more patient-present shared antigens in full-length and one or more parts of one or more patient-present shared cancer antigens. Examples include:
antigenic units comprising one patient-present shared antigen in full-length and one or more epitopes of one patient-present shared cancer antigen; and
antigenic units comprising several patient-present shared cancer antigens, each of them in full-length and one or more epitopes of one patient-present shared cancer antigen; and
antigenic units comprising one patient-present shared antigen in full-length and one or more epitopes of several patient-present shared cancer antigens; and
antigenic units comprising several patient-present shared cancer antigens, each of them in full-length and one or more epitopes of several patient-present shared cancer antigens.

In a preferred embodiment, the aforementioned epitopes are already known to be immunogenic, e.g. have been described to be immunogenic in the literature, or have already been predicted to bind to the patient's HLA class I and class II alleles, e.g. as described in the literature, preferably have already been predicted to bind to the patient's HLA class I alleles. In another preferred embodiment, the immunogenicity of the aforementioned epitopes is predicted, e.g. the binding of the epitopes to one or more of the patient's HLA class I and/or HLA class II molecules is predicted by methods known in the art, such as those disclosed in WO 2021/205027 A1, the disclosures of which is incorporated herein by reference, or those described herein, including those described in the section “Methods for designing an antigenic unit of an individualized construct”.

In some embodiments, the antigenic unit comprises 1 to 10 patient-present shared antigens in full-length.

In some other embodiments, the antigenic unit comprises 1 to 30 parts of one or more patient-present shared antigens, wherein these parts include multiple epitopes that are predicted to bind to a patient's HLA class I or class II alleles. In yet some other embodiments, the antigenic unit comprises 1 to 50 patient-present shared cancer epitopes, preferably epitopes that are known to or predicted to bind to the patient's HLA class I or class II alleles.

### Antigenic Units of Individualized Constructs Comprising One or More Patient-Present Shared Cancer Antigens or Parts Thereof and One or More Neoantigens or Parts Thereof

In further embodiments, the antigenic units are a combination of all of the afore-described embodiments relating to antigenic units, which comprise one or more patient-present shared cancer antigens or parts thereof and all of the afore-described embodiments relating to antigenic units, which comprise one or more neoantigens or parts thereof.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more patient-present shared cancer antigens or parts thereof and one or more neoantigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more patient-present shared cancer antigens or parts thereof and one or more neoantigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more patient-present shared cancer antigens or parts thereof and one or more neoantigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some the disclosure relates to a construct, the construct being:
(i) a polypeptide comprising a targeting unit selected from the group consisting of mutated human CCL3 and mutated human CCL3L1, a multimerization unit and an antigenic unit comprising one or more patient-present shared cancer antigens or parts thereof and one or more neoantigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

Antigenic units comprising one or more patient-present shared cancer antigens or parts thereof and optionally one or more neoantigens and parts thereof are described in detail in WO 2021/205027A1, the content of which is included herein by reference. Any of such antigenic units can be used as antigenic unit in the constructs disclosed herein for use in individualized anticancer therapy.

### Methods for Designing an Antigenic Unit of an Individualized Constructs

The patient-present shared cancer antigens and neoantigens identified in a particular patient are preferably further processed to find those antigens that will render the constructs most effective, when those antigens are included into the antigenic unit. The way and order in which such processing is done depends on how said antigens were identified, i.e. the data that form the basis for such processing.

In some embodiments, the processing and selecting of the antigen(s) to be included in the antigenic unit is carried out as follows:
1) A search in the literature and/or in one or more databases is carried out to retrieve information about and sequences of shared cancer antigens and preferably information about their expression pattern, immunogenicity or predicted immunogenicity, epitopes and HLA presentation. Such search is also carried out to determine whether the identified antigen is a patient-present shared cancer antigen or a neoantigen.
2) If it was determined that the identified antigen is a patient-present shared cancer antigen, the sequence thereof is studied to identify epitopes, preferably all epitopes, that are predicted to bind to the patient's HLA class I/II alleles. The prediction may be carried out by in silico methods, using prediction tools known in the art, e.g. prediction software known in the art, such as NetMHCpan and similar software.
3) The most promising sequences of the patient-present shared cancer antigen which are most immunogenic or predicted to be most immunogenic, including those that show predicted binding to one or more of the patient's HLA class I/II alleles, are selected for inclusion into the antigenic unit. In some embodiments, minimal epitopes are selected, e.g. if only a few promising epitopes were identified in step 2 or if longer stretches of non-immunogenic sequences are present between the epitopes. In some other embodiments, a longer sequence is selected which comprises several epitopes that bind to the patient's specific HLA alleles. In yet some other embodiments, the full-length sequence of the antigen is selected for inclusion into the antigenic unit.
4) The most promising parts of neoantigen sequences, e.g. neoepitopes, are selected for inclusion into the antigenic unit based on predicted immunogenicity and binding to the patient's HLA class I/II alleles of such sequences.

Tumor mutations are discovered by sequencing of tumor and normal tissue and comparing the obtained sequences from the tumor tissue to those of the normal tissue. A variety of methods is available for detecting the presence of a particular mutation or allele in a patient's DNA or RNA. Such methods include dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. Alternatively, mutations may be identified by direct protein sequencing.

Out of the maybe hundreds or thousands of mutations in the tumor exome, in some embodiments, the most promising sequences are selected in silico based on predictive HLA-binding algorithms. The intention is to identify all relevant epitopes and after a ranking or scoring, determine the sequences to be included in the antigenic unit. Methods known in the art may suitable for scoring, ranking and selecting neoepitopes include those disclosed in WO 2020/065023A1 and WO 2020/221/783A1.

Further, any suitable algorithm for such scoring and ranking may be used, including the following:

Available free software analysis of peptide-MHC binding (IEDB and NetMHCpan) that can be downloaded from the following websites:
www.iedb.org/
www.cbs.dtu.dk/services/NetMHC/

Commercially available advanced software to predict optimal sequences is available e.g. from:
www.oncoimmunity.com/
omictools.com/t-cell-epitopes-category
github.com/griffithlab/pVAC-Seq
crdd.osdd.net/raghava/cancertope/help.php
www.epivax.com/tag/neoantigen/

In some embodiments, each mutation is scored with respect to its immunogenicity, and the most immunogenic patient-specific shared cancer epitopes and/or neoepitopes are selected and optimally arranged in the antigenic unit.

Antigenic Unit of Non-Individualized Constructs for Use in Off-the Shelf Anticancer treatments

A non-individualized or “off-the-self” construct (also referred to as a construct comprising shared cancer antigen(s)) comprises an antigenic unit, which comprises one or more shared cancer antigens or parts thereof.

“Shared cancer antigen” or “shared tumor antigen” is used herein to describe an antigen that is known (e.g. that has been described in the literature) to be expressed by many tumors, either across patients with the same cancer type, or across patients and cancer types, and includes shared tumor-associated antigens and shared tumor-specific antigens.

“Shared cancer epitope” is a part of a shared cancer antigen used herein to describe an amino acid sequence, or a nucleic acid sequence encoding same, comprised in a shared cancer antigen, which is known to be immunogenic (e.g. that has been described to be immunogenic in the literature) or which has been predicted to be immunogenic.

In some embodiments, the antigenic unit of non-individualized constructs for use in the treatment of cancer comprises one or more shared cancer antigens or parts thereof, e.g. shared cancer epitopes, which are known to be immunogenic, have known expression patterns and/or are known or have already been predicted to bind to specific HLA class I and class II molecules.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more shared cancer antigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more shared cancer antigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more shared cancer antigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more shared cancer antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

Some shared cancer antigens are proteins comprising an amino acid sequence that comprise one or more mutations, i.e. shared cancer epitopes which are known to be immunogenic or which have been predicted to be immunogenic. Other patient-present shared cancer antigens are proteins which do not comprise mutations, but which are overexpressed or expressed in tissues where they are normally not expressed.

In some embodiments, the shared cancer antigen is selected from the group consisting of overexpressed cellular proteins, aberrantly expressed cellular proteins, cancer testis antigens, differentiation antigens, viral antigens, mutated oncogenes, mutated tumor suppressor genes, oncofetal antigens, shared fusion antigens, shared intron retention antigens, shared antigens caused by frameshift mutation, dark matter antigens and shared antigens caused by spliceosome mutations.

Examples of shared cancer antigens for use in the antigenic unit of non-individualized constructs are disclosed in the section “Antigenic unit of individualized constructs comprising one or more patient-present shared cancer antigens or parts thereof” herein, and any of these shared cancer antigens may be used for inclusion into the antigenic unit of non-individualized constructs.

Any shared cancer antigen sequence of sufficient length that includes a specific epitope may be used as the antigenic unit. Accordingly, in some embodiments, the antigenic unit comprises an amino acid sequence of at least 7 amino acids, such as at least 8 amino acids, corresponding to at least about 21 nucleotides, such as at least 24 nucleotides, in a nucleic acid sequence encoding such antigenic unit.

In yet some other embodiments, the antigenic unit comprises one or more parts of a shared cancer antigen, e.g. one or more shared cancer epitopes. In yet some other embodiments, the antigenic unit comprises one or more parts of several shared cancer antigens, e.g. one or more epitopes of several shared cancer antigens. In yet some other embodiments, the antigenic unit comprises one or more shared antigens in full-length and one or more parts of one or more shared cancer antigens. Examples include:
antigenic units comprising one shared antigen in full-length and one or more epitopes of one shared cancer antigen; and
antigenic units comprising several shared cancer antigens, each of them in full-length and one or more epitopes of one shared cancer antigen; and
antigenic units comprising one shared antigen in full-length and one or more epitopes of several shared cancer antigens; and
antigenic units comprising several shared cancer antigens, each of them in full-length and one or more epitopes of several shared cancer antigens.

Examples of polynucleotides/polypeptides/dimeric proteins comprising HPV shared cancer antigens are disclosed in WO 2013/092875A1, the content of which is incorporated herein by reference.

### Methods for Designing an Antigenic Unit of Construct Comprising Shared Cancer Antigen(s)

Also for constructs comprising shared cancer antigen(s), the antigenic unit is preferably designed to include those sequences that are likely to render the construct effective in a variety of patients, e.g. patients having a certain type of cancer.

In some embodiments, the selection of the antigen to be included in the antigenic unit is carried out by performing a search in the literature and/or in one or more databases to retrieve information about and sequences of shared cancer antigens and preferably information about their expression pattern, observed or predicted immunogenicity, epitopes and/or HLA presentation. Epitopes are then identified that are known or predicted to bind to a variety of HLA class I/II alleles of many patients or that bind a certain subset of HLA class I/II alleles which is dominant in a certain type of cancer and/or a certain patient population across different types of cancer. Preferably, the most promising sequences, i.e. the sequences of the shared cancer antigens which are most immunogenic or predicted to be most immunogenic, are selected for inclusion into the antigenic unit.

### Antigenic Units of Constructs Comprising One or More Infectious Antigens or Parts Thereof

In another aspect, the constructs disclosed herein comprise an antigenic unit, which is designed for the treatment of an infectious disease and the construct is for use in the treatment of an infectious disease.

In some embodiments, the antigenic unit comprised in such constructs comprises one or more antigens or parts thereof which are relevant for infectious diseases, i.e. one or more infectious antigens, i.e. antigens or parts thereof derived from pathogens.

“Infectious disease” is used herein to describe a condition caused by a pathogen or a condition wherein a pathogen is involved in causing it. An example of the latter are eggs of a parasite, which do not cause the disease itself but develop into larvae which cause it.

“A pathogen” is an organism or agent that causes a disease or is involved in causing a disease. Pathogens include viruses, bacteria, fungi and parasites.

The antigens described in this section are “infectious antigens”, i.e. antigens derived from pathogens, i.e. they are comprised (or naturally found) in proteins of a pathogen which causes the disease or is involved in causing it. The terms “infectious antigen” and “antigen derived from a pathogen” may be used herein interchangeably.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more infectious antigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more infectious antigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more infectious antigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more infectious antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more antigens derived from one or more pathogens or parts of such antigens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more antigens derived from one or more pathogens or parts of such antigens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more antigens derived from one or more pathogens or parts of such antigens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more antigens derived from one or more pathogens or parts of such antigens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In the above-described embodiments, the antigenic unit comprises one or more antigens derived from a pathogen or parts of such antigens, e.g. one antigen derived from a pathogen several antigens derived from a pathogen, e.g. from the same or different proteins of such pathogen.

In some embodiments, the antigenic unit comprises one or more antigens derived from multiple pathogens or parts of such antigens. In some embodiments, the multiple pathogens are multiple different pathogens. In that context, a “different pathogen” may, for example, be a different virus or bacterium or a different strain of the same virus or bacterium or it may be the same strain, but comprising one or more mutations.

A construct comprising one or more antigens or parts thereof derived from multiple pathogens may be for use in a pan-vaccine, e.g. a vaccine targeting different (seasonal) viruses. For example, the pan-vaccine could target betacoronavirus and influenza or target different strains of e.g. betacoronaviruses or different mutations of the same strain.

Examples of infectious antigens/antigens that are derived from pathogens are such of bacterial origin, e.g. tuberculosis antigens and OMP31 from brucellosis, or viral origin, e.g. HIV antigens like e.g. gp120, glycoprotein D from HSV-2, influenza virus antigens like hemagglutinin, nucleoprotein and M2, RSV antigens like the F protein or the G protein, and HPV antigens such as E1, E2, E6, E7, L1 or L2, such as E6 and E7 of HPV16 or HPV18.

In some embodiments, the antigenic unit comprises one or more betacoronavirus antigens or parts thereof.

Betacoronaviruses denotes a genus in the subfamily Orthocoronaviridae.

Betacoronaviruses are enveloped, positive-sense single-stranded RNA viruses. Within the genus, four lineages are commonly recognized: lineage A (subgenus Embecovirus), lineage B (subgenus Sarbecovirus), lineage C (Merbecovirus) and lineage D (Nobecovirus). Betacoronaviruses include the following viruses which caused/cause epidemics/pandemics in humans or can infect humans: SARS-CoV, which causes severe acute respiratory syndrome (SARS), MERS-CoV, which causes Middle East respiratory syndrome (MERS), SARS-CoV-2, which causes coronavirus disease 2019 (Covid-19), HCoV-OC43 and HCoV—HKU1. SARS-CoV and SARS-CoV-2 belong to the lineage B (subgenus Sarbecovirus), MERS-CoV belongs to the lineage C (Merbecovirus) and HCoV-OC43 and HCoV—HKU1 belong to the lineage A (subgenus Embecovirus).

In some embodiments, the antigen is the spike protein of SARS-CoV or SARS-CoV-2, or a part thereof.

In some embodiments, the antigenic unit comprises one or more antigens or parts thereof derived from one or more pathogens selected from the list consisting of influenza virus, Herpes simplex virus, CMV, RSV, HPV, HBV, brucella bacteria, HIV, HSV-2 and Mycobacterium tuberculosis bacteria.

The constructs disclosed herein for use in the treatment of infectious diseases are ideally suited for fighting pandemics and epidemics, as they can induce rapid, strong immune responses. Such immune responses are induced through inclusion into the antigenic unit of one or more full-length infectious antigens or a part of one or more infectious antigens, such parts may for example be selected T cell epitopes, or through combinations thereof.

In some embodiments, the constructs can be used in a prophylactic setting or a therapeutic setting or both a prophylactic and a therapeutic setting.

Antigenic Units of Constructs Comprising One or More T Cell Epitopes from One or More Infectious Antigens

In some embodiments, the antigenic unit of a construct disclosed herein for use in the treatment of an infectious disease comprises at least one T cell epitope from one or more infectious agents/derived from one or more pathogens. Such T cell epitopes are comprised (or naturally found) in proteins of pathogens. Conserved parts of the genome among many pathogens comprise T cell epitopes capable of initiating immune responses.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising at least one T cell epitope from one or more infectious antigens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising at least one T cell epitope from one or more infectious antigens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising at least one T cell epitope derived from one or more pathogens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising at least one T cell epitope derived from one or more pathogens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising at least one T cell epitope derived from one or more pathogens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising at least one T cell epitope derived from one or more pathogens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the antigenic unit comprises at least one T cell epitope of a pathogen, i.e. one T cell epitope of a pathogen or more than one T cell epitope of a pathogen, i.e. multiple T cell epitopes of a pathogen. In some embodiments, the multiple T cell epitopes are of the same pathogen, i.e. (naturally) comprised in the same or different proteins of the pathogen. In other embodiments, the multiple T cell epitopes are of multiple different pathogens, i.e. (naturally) comprised in protein of different pathogens.

The at least one T cell epitope comprised in the antigenic unit has a length of from 7 to about 200 amino acids, with a longer T cell epitope possibly including hotspots. As mentioned earlier, a “hotspot” is a region that contains several minimal T cell epitopes (e.g. having a length of from 7-15 amino acids) that are predicted to be presented by different HLA alleles to cover a broad range of world population.

In some embodiments, the antigenic unit comprises at least one T cell epitope with a length of from 7 to 150 amino acids, preferably of from 7 to 100 amino acids, e.g. from about 10 to about 100 amino acids or from about 15 to about 100 amino acids or from about 20 to about 75 amino acids or from about 25 to about 50 amino acids.

A T cell epitope having a length of about 60 to 200 amino acids may be split into shorter sequences and included into the antigenic unit separated by linkers, e.g. linkers as described herein. By way of example, a T cell epitope having a length of 150 amino acids may be split into 3 sequences of 50 amino acids each, and included into the antigenic unit, with linkers separating the 3 sequences from each other.

In some embodiments, the antigenic unit comprises multiple T cell epitopes which are separated from each other by linkers, e.g. linkers as discussed herein.

In some embodiments, the at least one T cell epitope has a length suitable for presentation by MHC. Thus, in some embodiments, the antigenic unit comprises at least one T cell epitope having a length suitable for specific presentation on MHC class I or MHC class II. In some embodiments, the at least one T cell epitope has a length of from 7 to 11 amino acids for MHC class I presentation. In other embodiments, the at least one T cell epitope has a length of about 15 amino acids for MHC class II presentation.

The number of T cell epitopes in the antigenic unit may vary, and depends on the length and number of other elements included in the antigenic unit, e.g. linkers.

In some embodiments, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.

In a preferred embodiment, the at least one T cell epitope is from a conserved region of the pathogen, i.e. is conserved between several subgenera, species or strains of a respective pathogen.

The T cell epitopes may be comprised in any of the pathogen's proteins, i.e. in surface proteins but also in the internal proteins such as viral nucleocapsid proteins or viral replicase polyproteins or in other structural and non-structural proteins.

A construct comprising an antigenic unit comprising T cell epitopes from conserved regions of pathogens will provide protection against several species/strains of the pathogen. Such a construct will also provide protection against multiple variants of a pathogen, which is important for its efficacy against future mutated pathogens. Viruses are known to mutate, e.g. undergo viral antigen drift or antigen shift. The finding of conserved regions across a viral genus makes it likely that these conserved regions are needed to maintain essential structures or functions, thus it is anticipated that future mutations will take place in the less-conserved regions. By raising an immune response against the conserved regions, the individual treated a construct as disclosed herein will be protected also against mutated (and thus novel) strains of the future.

In some embodiments, the antigenic unit is therefore designed to evoke a cell-mediated immune response through activation of T cells against the T cell epitopes of the infectious antigen/from a pathogen included in such antigenic unit. T cells recognize epitopes when they have been processed and presented complexed to an MHC molecule.

In some embodiments, the T cell epitope is known in the art, e.g. one that has been studied and described in the literature, e.g. known to be immunogenic, e.g. its immunogenicity has been confirmed by appropriate methods and the results have been published, e.g. in a scientific publication. In some embodiments, the antigenic unit includes multiple T cell epitopes that are known to be immunogenic.

For example, useful T cell epitopes known in the art are those against infection by SARS-CoV2 in humans can be found in Grifoni et al., Cell Host Microbe. 2021 Jul. 14; 29(7): 1076-1092. Such T cell epitopes may thus be included in the antigenic unit of vectors for use in treating SARS-CoV2 in humans. Another example of such T cell epitopes is the T cell epitope with the sequence CTELKLSDY (SEQ ID NO: 20) of the nucleoprotein from influenza A virus, the T cell epitope with the sequence NLVPMVATV (SEQ ID NO: 21) of the 65 kDa phosphoprotein from human herpesvirus 5 (human cytomegalovirus) and the T cell epitope with the sequence KLVANNTRL (SEQ ID NO: 22) of diacylglycerol acyltransferase/mycolyltransferase Ag85B from Mycobacterium tuberculosis.

As an example, the at least one T cell epitope may be a part of the sequence of proteins from SARS-CoV2, including the spike protein, the membrane protein, the envelope protein, the nucleocapsid protein, the ORF1a/b and the ORF3a protein. In some other embodiments, the T cell epitope is part of the following SARS-CoV2 genes/proteins: NCAP, AP3A, spike, ORF1a/b, ORF3a, VME1 and VEMP.

As an example, the at least one T cell epitope may be from a region of a human papilloma virus (HPV), e.g. from HPV16 or HPV18, e.g. at least one T cell epitope comprised in HPV antigens from the group consisting of E1, E2, E6, E7, L1 and L2, e.g. E6 and/or E7 of HPV16 and/or HPV18. By including such T cell epitopes in the constructs of the disclosure, such constructs may be for use in the treatment of HPV infections, e.g. provide protection against HPV. HPV infections are involved in certain cancers, such as squamous cell carcinoma of the head and neck, cervical cancer and vulvar squamous cell carcinoma.

As another example, the at least one T cell epitope may be from a region of a human influenza virus, such as human influenza virus A, human influenza virus B, human influenza virus C and human influenza virus D. As an example, the human influenza virus may be a specific hemagglutinin (HA) subtype, such as H1, H2, and H3, and/or a specific neuraminidase (NA) subtype, such as N1 or N5. As an example, the human influenza virus may be a H1N1 subtype. Such T cell epitopes may thus be included in the antigenic unit of a construct of the disclosure for use in the treatment of influenza infections.

In some other embodiments, the at least one T cell epitope is predicted to be immunogenic, e.g. is selected based on the predicted ability to bind to HLA class I/II alleles. In some embodiments, the antigenic unit includes multiple T cell epitopes that are predicted to bind to HLA class I/II alleles. The T cell epitopes are selected in silico on the basis of predictive HLA-binding algorithms, e.g. algorithms that are known in the art. After having identified all relevant epitopes, the epitopes are ranked according to their ability to bind to HLA class I/II alleles and the epitopes that are predicted to bind best are selected to be included in the antigenic unit.

In yet some other embodiments, the antigenic unit comprises multiple T cell epitopes some of which are known to be immunogenic and others that are predicted to be immunogenic. In some embodiments, the T cell epitopes are separated from each other by linkers. In some embodiments, some of the T cell epitopes are separated by linkers. In some other embodiments, all T cell epitopes are separated from each other by linkers. Suitable linkers are disclosed herein.

Antigenic units comprising T cell epitopes for use in a construct as disclosed herein for the prophylactic and therapeutic treatment of betacoronavirus infections and generally applicable methods for selecting T cell epitopes for constructs as disclosed herein for use in the prophylactic and therapeutic treatment of infectious diseases are disclosed in detail in WO2021/219897A1, the disclosures of which is incorporated herein by reference.

Antigenic Units of Constructs Comprising One or More Full-Length Infectious Antigens, or Parts Thereof or One or More B Cell Epitopes from One or More Infectious Agents

In some embodiments, a subject, e.g. a human individual, is a healthy individual and the constructs disclosed herein are used prophylactically, e.g. to prevent a disease. Typically, the constructs will be used to induce immunity in individuals where it is desired to raise neutralizing antibodies against a pathogen in a prophylactic setting, e.g. to prevent an infection.

In some embodiments, the constructs disclosed herein comprise an antigenic unit comprising at least one protein derived from a pathogen which is a full-length protein or a part thereof, i.e., a full-length infectious antigen, or a part thereof. In some embodiments, the protein is a full-length surface protein or a part thereof, e.g. a full-length viral surface protein or a full-length bacterial surface protein or a full-length surface protein of any other pathogen. In some other embodiments, the protein is a full-length bacterial protein which is secreted by the bacterium, e.g. secreted into the cytoplasm of infected subjects.

In some other embodiments, the antigenic unit comprises more than one full-length infectious antigen or parts thereof, e.g. multiple full-length infectious antigens or multiple parts of a full-length infectious antigen or multiple parts of multiple full-length infectious antigens.

Alternatively, the antigenic unit comprises one or more full-length antigens derived from a pathogen or from multiple pathogens. In yet some other embodiments, the antigenic unit comprises a part of one or more full-length antigens derived from a pathogen or from multiple pathogens. In yet some other embodiments, the antigenic unit comprises multiple parts of one or more full-length antigens derived from a pathogen or from multiple pathogens.

In some embodiments, the antigenic unit comprises at least one infectious antigen which is a full-length protein of a betacoronavirus, such as the envelope protein, the spike protein, the membrane protein and, if the betacoronvirus is an Embecovirus, the spike-like protein hemagglutinin esterase.

In other embodiments, the antigenic unit comprises a part of a full-length protein derived from a pathogen, such as the RBD domain of the spike protein of SARS-CoV-2 or the head or stem domain of hemagglutinin of the influenza virus or the head-only part of the RSV F protein.

In other embodiments, the antigenic unit comprises multiple parts of one infectious antigen. In other embodiments, the antigenic unit comprises one part of several infectious antigens, e.g. one part of infectious antigen 1 and one part of infectious antigen 2 and 1 part of infectious antigen 3. In other embodiments, the antigenic unit comprises several parts of several infectious antigens, e.g. 2 parts of infectious antigen 1 and 3 parts of infectious antigen 2. The infectious antigens 1, 2 and 3 may be derived from one pathogen or from multiple, different pathogens.

If more than one infectious antigen is comprised in the antigenic unit, or more than 1 part of one or more infectious antigens, some or all of the antigens or some or all parts thereof may be separated by linkers, e.g. by linkers as disclosed herein.

The one or more full-length infectious antigens or parts thereof typically comprise conformational B cell epitopes, but may also comprise linear B cell epitopes and/or T cell epitopes. In contrary to the T cell epitopes discussed in the previous section herein, these T cell epitopes are not isolated, but are presented to the immune system in their natural environment, i.e. flanked by the amino acid residues which are present in the antigen.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more full-length infectious antigens or parts thereof; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more full-length infectious antigens or parts thereof, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more full-length infectious antigens or parts thereof; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more full-length infectious antigens or parts thereof, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising one or more full-length antigens derived from one or more pathogens or parts of such full-length antigens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising one or more full-length antigens derived from one or more pathogens or parts of such full-length antigens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more full-length antigens derived from one or more pathogens or parts of such full-length antigens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising one or more full-length antigens derived from one or more pathogens or parts of such full-length antigens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the antigenic unit comprises at least one B cell epitope derived from a pathogen, e.g. at least one B cell epitope comprised in a protein of a pathogen, such as a surface protein or any of the aforementioned proteins. In some other embodiments, the antigenic unit comprises multiple B cell epitopes derived from a pathogen, e.g. comprised in one or more proteins of a pathogen. In some embodiments, some or all of the multiple B cell epitopes are separated by linkers, e.g. linkers as disclosed herein. The at least one B cell epitope may be a linear or a conformational B cell epitope.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising at least one B cell epitope derived from one or more pathogens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising at least one B cell epitope derived from one or more pathogens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising at least one B cell epitope derived from one or more pathogens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising at least one B cell epitope derived from one or more pathogens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

Once administered, the constructs comprising an antigenic unit comprising one or more full-length infectious antigens, or parts of such antigens, elicit a B cell response and T cell response and can be used prophylactic or therapeutic.

Such antigens may be selected for inclusion into the antigenic unit according to their known or predicted therapeutic efficacy, see e.g. WO2021/219897A1, the disclosures of which is incorporated herein by reference.

Antigenic Units of Constructs Comprising B Cell Epitopes and T Cell Epitopes from One or More Infectious Antigens

In some embodiments, the constructs disclosed herein will, once administered to a subject, elicit a T cell response and a B cell response. In a pandemic or an epidemic situation, it is neither time-efficient to first diagnose individuals to determine if they primarily need a B or T cell response, nor to determine whether prophylactic or therapeutic treatment is the highest medical need. Less so, as the determination of whether or not an individual is infected can be difficult due to lack of (sufficient) applicable tests. Thus, being able to protect and cure at the same time is important. By combining both full-length infectious antigens or parts thereof, such as B cell epitopes, and T cell epitopes, such as conserved T cell epitopes from infectious antigens, both a strong humoral and cellular response is elicited once the constructs disclosed herein comprising such full-lengths antigens/B cell epitopes/T cell epitopes are administered to a subject.

The response can be more humoral or more cellular, depending on the composition of the antigenic unit.

Thus, in some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising (a) one or more full-length infectious antigens or parts of such antigens and (b) at least one T cell epitope from one or more infectious antigens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising (a) one or more full-length infectious antigens or parts of such antigens and (b) at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising (a) one or more full-length infectious antigens or parts of such antigens and (b) at least one T cell epitope from one or more infectious antigens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising (a) one or more full-length infectious antigens or parts of such antigens and (b) at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising (a) one or more full-length antigens or parts thereof and (b) at least one T cell epitope, wherein the one or more antigens and at least one T cell epitope are derived from one or more pathogens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising (a) one or more full-length antigens or parts thereof and (b) at least one T cell epitope, wherein the one or more antigens and at least one T cell epitope are derived from one or more pathogen, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising (a) one or more full-length antigens or parts thereof and (b) at least one T cell epitope, wherein the one or more antigens and at least one T cell epitope are derived from one or more pathogens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising (a) one or more full-length antigens or parts thereof and (b) at least one T cell epitope, wherein the one or more antigens and at least one T cell epitope are derived from one or more pathogen, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

Such a combination of T cell epitopes and infectious antigens or parts thereof may be selected for inclusion into the antigenic unit according to the known or predicted immunogenicity of the infectious antigen or part thereof and the T cell epitopes' known or predicted immunogenicity, e.g. see WO2021/219897A1, the disclosures of which is incorporated herein by reference.

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising (a) one or more B cell epitopes from one or more infectious antigens and (b) at least one T cell epitope from one or more infectious antigens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising (a) one or more B cell epitopes from one or more infectious antigens and (b) at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising one or more B cell epitopes from one or more infectious antigens and (b) at least one T cell epitope from one or more infectious antigens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising (a) one or more B cell epitopes from one or more infectious antigens and (b) at least one T cell epitope from one or more infectious antigens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1 and an antigenic unit comprising (a) one or B cell epitopes and (b) at least one T cell epitope, wherein the one or more B cell epitopes and the at least one T cell epitope are derived from one or more pathogens; or
(ii) a polypeptide encoded by the nucleotide sequence as defined in (i);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1 and an antigenic unit comprising (a) one or B cell epitopes and (b) at least one T cell epitope, wherein the one or more B cell epitopes and the at least one T cell epitope are derived from one or more pathogens, wherein the targeting unit comprises one or more mutations, compared to the wild type, which reduce or prevent the formation of targeting unit oligomers; or
(ii) a polynucleotide encoding the polypeptide as defined in (i).

In some embodiments, the disclosure relates to a construct, the construct being:
(i) a polynucleotide comprising a nucleotide sequence encoding a targeting unit selected from the group consisting of mutated human CCL3 and mutated human CCL3L1, a multimerization unit and an antigenic unit comprising (a) one or B cell epitopes and (b) at least one T cell epitope, wherein the one or more B cell epitopes and the at least one T cell epitope are derived from one or more pathogens; or
(ii) a polypeptide encoded by the nucleotide sequence as defined in (i); or
(iii) a multimeric protein consisting of multiple polypeptides as defined in (ii);
wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a construct, the construct being:
(i) a polypeptide comprising a targeting unit selected from the group consisting of mutated human CCL3 and mutated human CCL3L1, a multimerization unit and an antigenic unit comprising (a) one or B cell epitopes and (b) at least one T cell epitope, wherein the one or more B cell epitopes and the at least one T cell epitope are derived from one or more pathogens, wherein the targeting unit comprises one or more mutations, compared to the wild type, which reduce or prevent the formation of targeting unit oligomers; or
(ii) a polynucleotide encoding the polypeptide as defined in (i); or
(iii) a multimeric protein consisting of multiple polypeptides as defined in (i).

In some embodiments, the full-lengths infectious antigens/parts thereof/B cell epitopes and the at least one T cell epitope are arranged in the antigenic unit as follows: the at least one T cell epitope is arranged in a subunit which is connected to the targeting unit or to the multimerization unit by a first linker, such as a unit linker. If multiple T cell epitopes are present in the subunit, the T cell epitopes are preferably separated by subunit linkers. Further, the subunit is separated from the one or more full-length infectious antigens/parts thereof/B cell epitopes by a second linker. Thus, the subunit with the T cell epitope(s) is closest to the targeting unit (or multimerization unit, if present), while the infectious antigen(s) or parts thereof or B cell epitopes constitute the C-terminal end of the polypeptide/multimeric protein.

The subunit linkers, first linker/unit linker and second linker may be linkers as discussed herein, e.g. as discussed in the “Linkers in the antigenic unit” and “Unit linker” sections herein.

### Further Embodiments of the Antigenic Unit

The following applies in general to the antigenic unit in constructs disclosed herein.

The term “antigen” is used in this section and the following “Linkers” section for a neoantigen, a neoepitope, a patient-present shared cancer antigen, a part of a patient-present shared cancer antigen, such as a patient-present shared cancer epitope, a shared cancer antigen, a part of a shared cancer antigen, such as a shared cancer epitope, an infectious antigen/antigen derived from a pathogen or a part thereof, a B cell epitope of an infectious antigen/antigen derived from a pathogen and a T cell epitope of an infectious antigen/antigen derived from a pathogen.

In some embodiments, the antigenic unit comprises only one copy of each antigen. In some other embodiments, the antigenic unit comprises multiple copies of one antigen or multiple copies of several different antigens.

In some embodiments, the antigenic unit comprises only one copy of each antigen, so that when e.g. 10 different antigens are comprised in the antigenic unit, a construct comprising said antigenic unit may elicit an immune response against all 10 different antigens and thus attack the cancer efficiently, if said antigens are cancer-relevant antigens.

In some other embodiments, if e.g. only a few neoepitopes could be identified in a specific patient that are predicted to be sufficiently immunogenic/predicted to bind to the patient's HLA alleles, then the antigenic unit may comprise at least two copies of a particular antigen, e.g. particular neoepitope, in order to strengthen the immune response to the antigen. If in such patient one or more patient-present shared cancer antigens are identified in addition to such few neoepitopes, it is however preferred to then include such one or more patient-present shared cancer antigens or parts thereof into the antigenic unit rather than including multiple copies of the same neoepitope.

The length of the antigenic unit (i.e. number of amino acids) is determined by the length of the antigen(s) comprised therein as well as their number.

In some embodiments, the antigenic unit comprises up to 3500 amino acids, such as from 60 to 3500 amino acids, e.g. from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In order to enhance the immune response, particularly for a construct comprising neoantigens/neoepitopes, the antigens may be arranged in the antigenic unit as described in the following paragraphs.

The antigenic unit can be described as a polypeptide having an N-terminal start and a C-terminal end. If the construct comprises a multimerization unit, the N-terminal start of the antigenic unit is connected to the multimerization unit, e.g. via a linker, such as a unit linker. If the construct does not comprise a multimerization unit, the N-terminal start of the antigenic unit is connected to the targeting unit, e.g. via a linker, such as a unit linker.

In some embodiments, the antigens, preferably epitopes, are arranged in the order of more antigenic (immunogenic) to less antigenic in the direction from the N-terminal start of the antigenic unit to its C-terminal end. In some other embodiments, particularly if the hydrophilicity/hydrophobicity varies greatly among the antigens, the most hydrophobic antigen(s) is/are substantially positioned in the middle of the antigenic unit and the most hydrophilic antigen(s) is/are positioned at the N-terminal start and/or the C-terminal end of the antigenic unit.

Since a true positioning in the middle of the antigenic unit is only possible if the antigenic unit comprises an odd number of antigens, the term “substantially” in this context refers to antigenic units comprising an even number of antigens, wherein the most hydrophobic antigens are positioned as closed to the middle as possible.

In yet some other embodiments, the antigens are arranged in the antigenic unit such that they alternate between a hydrophilic and a hydrophobic antigen.

In some embodiments, GC rich nucleic acid sequences encoding for antigens (e.g. GC rich nucleic acid sequences encoding neoepitopes or epitopes) are arranged in such a way, that GC clusters are avoided. In some embodiments, GC rich nucleic acid sequences encoding for antigens are arranged such that there is at least one non-GC rich nucleic acid sequence between them.

In some embodiments, the antigenic unit comprises one or more linkers. In some other embodiments, the antigenic unit comprises multiple antigens, e.g. multiple epitopes, e.g. neoepitopes, wherein some but not all of the antigens are separated by linkers. In yet some other embodiments, the antigenic unit comprises multiple antigens wherein each antigen is separated from other antigens by linker(s). An alternative way to describe the separation of each antigen from other antigens by linkers is that all but the terminal antigen, i.e. the antigen at the C-terminal end of the antigenic unit (i.e. the end of the antigenic unit that is not connected to the multimerization unit or targeting unit), are arranged in antigenic subunits, wherein each subunit comprises or consists of an antigen e.g. a neoepitope, and a subunit linker. Hence, an antigenic unit comprising n antigens comprises n−1 antigenic subunits, wherein each subunit comprises an antigen and a subunit linker, and further comprises a terminal antigen. In some embodiments, n is an integer of from 1 to 50, e.g. 3 to 50 or 15 to 40 or 10 to 30 or 10 to 25 or 10 to 20 or 15 to 30 or 15 to 25 or 15 to 20.

In some embodiments, the antigenic unit comprises B cell epitopes and T cell epitopes from an infectious antigen, e.g. a full-length infectious antigen or part thereof and one or more T cell epitopes from an infectious antigen and the antigenic unit is designed such that the T cell epitopes are arranged closest to the multimerization unit, if present, or the targeting unit and the infectious antigen is at the C-terminal end of the antigenic unit. In some embodiments, some or all of the T cell epitopes are separated by linkers and the infectious antigen is preferably separated from the “subunit” comprising the T cell epitopes by a linker. Such afore-mentioned antigenic unit designs are disclosed in WO 2022/233851A1, the disclosures of which is incorporated herein by reference.

### Linkers

The constructs disclosed herein and the elements or units comprised in such constructs, e.g. the antigenic unit may comprise linkers, e.g. linkers that separate some or all of the antigens comprised in the antigenic unit. As described earlier, if all antigens, such as neoepitopes, are separated from each other by linkers, another way to describe them/the antigenic unit is that they are arranged in subunits comprising the antigen and a subunit linker. In the following, the term subunit linker and linker are used interchangeably, and both denote a linker comprised in the constructs disclosed herein and the elements or units comprised in such constructs, e.g. the antigenic unit.

In some embodiments, the linkers are designed to be non-immunogenic. A linker may be a rigid linker, meaning that it does not allow the two amino acid sequences that it connects to substantially move freely relative to each other. Alternatively, it may be a flexible linker, i.e. a linker that allows the two amino acid sequences that it connects to substantially move freely relative to each other. Both types of linkers are useful.

In some embodiments, the linker is a flexible linker. A flexible linker in the antigenic unit allows for presenting the antigen(s) comprised therein in an optimal manner to e.g. T cells, even if the antigenic unit comprises a large number of antigens.

In some embodiments, the linker is a peptide consisting of from 4 to 40 amino acids, e.g. 35, 30, 25 or 20 amino acids, e.g. from 5 to 20 amino acids or 5 to 15 amino acids or 8 to 20 amino acids or 8 to 15 amino acids 10 to 15 amino acids or 8 to 12 amino acids. In some other embodiments, the linker consists of 10 amino acids.

In some embodiments, e.g. in an antigenic unit comprising multiple neoepitopes, the linker separating some or all of the neoepitopes is identical. If, however, one or more of the antigens comprise a sequence similar to that of the linker, it may be an advantage to substitute the neighboring linkers with a linker of a different sequence. Also, if an antigen-linker junction is predicted to constitute an immunogenic epitope, then a linker of a different sequence may be used.

In some embodiments, the linker is a flexible linker, preferably a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In some embodiments, the flexible linker is a serine (S) and/or glycine (G) rich linker, i.e. a linker comprising several serine and/or several glycine residues.

In the following, m is an integer from 1 to 5, e.g., 1, 2, 3, 4, or 5. In some embodiments, m is 2.

Preferred examples are GGGGS (SEQ ID NO: 23), GGGSS (SEQ ID NO: 24), GGGSG (SEQ ID NO: 25), GGSGG (SEQ ID NO: 26), SGSSGS (SEQ ID NO: 27) or multiple variants thereof such as GGGGSGGGGS (SEQ ID NO: 28), (GGGGS)m (SEQ ID NO: 29), (GGGSS)m (SEQ ID NO: 30), (GGSGG)m (SEQ ID NO: 31), (GGGSG)m (SEQ ID NO: 32) or (SGSSGS)m (SEQ ID NO: 33). In another preferred embodiment, the serine and/or glycine rich linker further comprises at least one leucine (L) residue, such as at least 1 or at least 2 or at least 3 leucine residues, e.g. 1, 2, 3 or 4 leucine residues.

In some embodiments, the linker comprises or consists of LGGGS (SEQ ID NO: 34), GLGGS (SEQ ID NO: 35), GGLGS (SEQ ID NO: 36), GGGLS (SEQ ID NO: 37) or GGGGL (SEQ ID NO: 38). In some other embodiments, the linker comprises or consists of LGGSG (SEQ ID NO: 39), GLGSG (SEQ ID NO: 40), GGLSG (SEQ ID NO: 41), GGGLG (SEQ ID NO: 42) or GGGSL (SEQ ID NO: 43). In yet some other embodiments, the linker comprises or consists of LGGSS (SEQ ID NO: 44), GLGSS (SEQ ID NO: 45) or GGLSS (SEQ ID NO: 46).

In yet some other embodiments, the linker comprises or consists of LGLGS (SEQ ID NO: 47), GLGLS (SEQ ID NO: 48), GLLGS (SEQ ID NO: 49), LGGLS (SEQ ID NO: 50) or GLGGL (SEQ ID NO: 51). In yet some other embodiments, the linker comprises or consists of LGLSG (SEQ ID NO: 52), GLLSG (SEQ ID NO: 53), GGLSL (SEQ ID NO: 54), GGLLG (SEQ ID NO: 55) or GLGSL (SEQ ID NO: 56). In yet some other embodiments, the linker comprises or consists of LGLSS (SEQ ID NO: 57), or GGLLS (SEQ ID NO: 58).

In some other embodiments, the linker is serine-glycine linker that has a length of 10 amino acids and comprises 1 or 2 leucine residues.

In some embodiments, the linker comprises or consists of LGGGSGGGGS (SEQ ID NO: 59), GLGGSGGGGS (SEQ ID NO: 60), GGLGSGGGGS (SEQ ID NO: 61), GGGLSGGGGS (SEQ ID NO: 62) or GGGGLGGGGS (SEQ ID NO: 63). In some other embodiments, the linker comprises or consists of LGGSGGGGSG (SEQ ID NO: 64), GLGSGGGGSG (SEQ ID NO: 65), GGLSGGGGSG (SEQ ID NO: 66), GGGLGGGGSG (SEQ ID NO: 67) or GGGSLGGGSG (SEQ ID NO: 68). In yet some other embodiments, the linker comprises or consists of LGGSSGGGSS (SEQ ID NO: 69), GLGSSGGGSS (SEQ ID NO: 70), GGLSSGGGSS (SEQ ID NO: 71), GGGLSGGGSS (SEQ ID NO: 72) or GGGSLGGGSS (SEQ ID NO: 73).

In a further embodiment, the linker comprises or consists of LGGGSLGGGS (SEQ ID NO: 74), GLGGSGLGGS (SEQ ID NO: 75), GGLGSGGLGS (SEQ ID NO: 76), GGGLSGGGLS (SEQ ID NO: 77) or GGGGLGGGGL (SEQ ID NO: 78). In some other embodiments, the linker comprises or consists of LGGSGLGGSG (SEQ ID NO: 79), GLGSGGLGSG (SEQ ID NO: 80), GGLSGGGLSG (SEQ ID NO: 81), GGGLGGGGLG (SEQ ID NO: 82) or GGGSLGGGSL (SEQ ID NO: 83). In yet some other embodiments, the linker comprises or consists of LGGSSLGGSS (SEQ ID NO: 84), GLGSSGLGSS (SEQ ID NO: 85) or GGLSSGGLSS (SEQ ID NO: 86).

In yet some other embodiments, the linker comprises or consists of GSGGGA (SEQ ID NO: 87), GSGGGAGSGGGA (SEQ ID NO: 88), GSGGGAGSGGGAGSGGGA (SEQ ID NO: 89), GSGGGAGSGGGAGSGGGAGSGGGA (SEQ ID NO: 90) or GENLYFQSGG (SEQ ID NO: 91). In yet some other embodiments, the linker comprises or consists of SGGGSSGGGS (SEQ ID NO: 92), SSGGGSSGGG (SEQ ID NO: 93), GGSGGGGSGG (SEQ ID NO: 94), GSGSGSGSGS (SEQ ID NO: 95), GGGSSGGGSG (SEQ ID NO: 12), GGGSSS (SEQ ID NO: 96), GGGSSGGGSSGGGSS (SEQ ID NO: 97) or GLGGLAAA (SEQ ID NO: 98).

In some other embodiments, the linker is a rigid linker. Such rigid linkers may be useful to efficiently separate sequences and prevent their interferences with each other, e.g. separate (larger) antigens and prevent their interferences with each other. In some embodiments, the linker comprises or consists of KPEPKPAPAPKP (SEQ ID NO: 99), AEAAAKEAAAKA (SEQ ID NO: 100), (EAAAK)m (SEQ ID NO: 101), PSRLEEELRRRLTEP (SEQ ID NO: 102) or SACYCELS (SEQ ID NO: 103).

In yet some other embodiments, the linker comprises or consists of TQKSLSLSPGKGLGGL (SEQ ID NO: 104). In yet some other embodiments, the inker comprises or consists of SLSLSPGKGLGGL (SEQ ID NO: 105).

In yet some other embodiments, the linker comprises or consists of GGSAGGSGSGSSGGSSGASGTGTAGGTGSGSGTGSG (SEQ ID NO: 106); or GGSGGGSEGGGSEGGGSEGGGSEGGGSEGGGSGGGS (SEQ ID NO: 107) or ELKTPLGDTTHT (SEQ ID NO: 108) or EPKSCDTPPPCPRCP (SEQ ID NO: 109).

In yet some other embodiments, the linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of suitable linkers are disclosed in paragraphs [0098]-[0099] and in the recited sequences of WO 2020/176797A1, in paragraphs [0135] to [0139] of US 2019/0022202A1, in WO 2017/118695 A1 and in WO 2021/219897A1, all of which are incorporated herein by reference.

### Unit Linker

In some embodiments, constructs disclosed herein comprise a unit linker. In some embodiments, the antigenic unit is connected to the targeting unit or multimerization unit by a unit linker. Thus, in some embodiments, constructs disclosed herein comprise a unit linker that connects the antigenic unit to the targeting unit or the antigenic unit to the multimerization unit. In some embodiments, the unit linker is a non-immunogenic linker and/or flexible or rigid linker.

In some embodiments, the unit linker comprises a restriction site. In some embodiments, the unit linker comprises or consists of/is GLGGL (SEQ ID NO: 51) or GLSGL (SEQ ID NO: 110). In some other embodiments, the unit linker comprises or consists of GGGGS (SEQ ID NO: 23), GGGGSGGGGS (SEQ ID NO: 28), (GGGGS)m (SEQ ID NO: 29), EAAAK (SEQ ID NO: 111), (EAAAK)m (SEQ ID NO: 101), (EAAAK)mGS (SEQ ID NO: 112), (EAAK)mGS (SEQ ID NO: 113), GPSRLEEELRRRLTEPG (SEQ ID NO: 114), AAY or HEYGAEALERAG (SEQ ID NO: 115).

### Signal Peptide

In some embodiments of the present disclosure, if the construct is a polynucleotide, such polynucleotide further comprises a nucleotide sequence which encodes a signal peptide. The signal peptide is located at the N-terminal end of the targeting unit. The signal peptide is designed to allow secretion of the polypeptide/multimeric protein from cells comprising the polynucleotide, e.g. cells having been transfected with a vector comprising the polynucleotide and is typically no longer present in the mature polypeptide/multimeric protein. Preferably, the signal peptide is that which is naturally present at the N-terminus of the targeting unit (also called the natural leader sequence).

In some embodiments, the targeting unit is mutated human CCL3, whose amino acid sequence comprises one or more mutations, compared to the amino acid sequence of the wild type, which reduce or prevent the formation of targeting unit oligomers. In some embodiments, a polynucleotide as disclosed herein comprising a nucleotide sequence encoding such targeting unit comprises a further nucleotide sequence that encodes a signal peptide that comprises an amino acid sequence having at least 85% sequence identity to the amino acid of SEQ ID NO: 116, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%. In some other embodiments, said signal peptide comprises the amino acid sequence of SEQ ID NO: 116, except that at the most three amino acids have been substituted, deleted or inserted, such as at the most two amino acids or such as at the most one amino acid. In some other preferred embodiments, said signal peptide comprises the amino acid sequence of SEQ ID NO: 116 and in yet some other preferred embodiments, said signal peptide consists of the amino acid sequence of SEQ ID NO: 116.

In some embodiments, the targeting unit is mutated human CCL3L1, whose amino acid sequence comprises one or more mutations, compared to the amino acid sequence of the wild type, which reduce or prevent the formation of targeting unit oligomers. In some embodiments, a polynucleotide as disclosed herein comprising a nucleotide sequence encoding such targeting unit further comprises a nucleotide sequence that encodes a signal peptide that comprises an amino acid sequence having at least 85% sequence identity to the amino acid of SEQ ID NO: 117, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%. In some other embodiments, said signal peptide comprises the amino acid sequence of SEQ ID NO: 117, except that at the most three amino acids have been substituted, deleted or inserted, such as at the most two amino acids or such as at the most one amino acid. In some other preferred embodiments, said signal peptide comprises the amino acid sequence of SEQ ID NO: 117. In yet some other preferred embodiments, said signal peptide c the amino acid sequence of SEQ ID NO: 117.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize at gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence of the polynucleotide or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the polypeptide, provided that the final proteins have the desired characteristics. For example, deletions, insertions, or substitutions of amino acid residues may produce a silent change and result in a functionally equivalent polypeptide.

Deliberate amino acid substitutions may be made based on similarity in polarity, charge, solubility, hydrophobicity, hydrophilicity, and/or the amphipathic nature of the residues as long as the desired properties of the protein in question are retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitutions such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, valine, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above, * indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form.

### Polynucleotides

In some embodiments, the construct disclosed herein is the polynucleotide.

Thus, a further aspect of the disclosure is a polynucleotide comprising a nucleotide sequence encoding a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the disclosure relates to a polynucleotide comprising a nucleotide sequence encoding a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

The polynucleotide may be a DNA or an RNA polynucleotide, including genomic DNA, cDNA and mRNA, either double-stranded or single-stranded, or synthetic DNA, such as DNA amplicons such as closed DNA strands, including doggybone DNA (Mucker et al., Vaccines 10(7), 2022, 1104) and linear DNA amplicons (Conforti et al., J Exp Clin Cancer Res 41(1), 2022, 195).

In some embodiments, the polynucleotide is optimized to the species of the subject to which it is administered. For administration to a human, in some embodiments, the nucleotide sequence of polynucleotide is human codon optimized.

### Vectors

In some embodiments, the polynucleotide is comprised in a vector.

Thus, a further aspect of the disclosure is a vector comprising a polynucleotide comprising a nucleotide sequence encoding a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the disclosure relates to a vector comprising a polynucleotide comprising a nucleotide sequence encoding a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

The vector may be any molecule which is suitable to deliver foreign nucleic acid sequences, such as DNA or RNA, into a cell (in vitro or in vivo) where they are expressed, i.e. expression vectors. In some embodiments, the vector is a plasmid, a virus, a cosmid, a phage, an artificial chromosome, such as a bacterial artificial chromosome or yeast artificial chromosome.

In some embodiments, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In some other embodiments, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

### Polycistronic Vectors

In some embodiments, the above-described vector is a polycistronic vector that allows the expression of the polypeptides disclosed herein and, in addition, the expression of one or more immunostimulatory compounds, as separate molecules.

A further aspect of the disclosure is a vector comprising:
(A) polynucleotide comprising a nucleotide sequence encoding a polypeptide comprising a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers; and
(B) one or more nucleic acid sequences encoding one or more immunostimulatory compounds, wherein the vector allows for the co-expression of the polypeptide and the one or more immunostimulatory compounds as separate molecules.

In some embodiments, the disclosure relates to a vector comprising:
(A) polynucleotide comprising a nucleotide sequence encoding a polypeptide comprising a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers; and
(B) one or more nucleic acid sequences encoding one or more immunostimulatory compounds,
wherein the vector allows for the co-expression of the polypeptide and the one or more immunostimulatory compounds as separate molecules.

Polycistronic vectors comprising a polynucleotide comprising a nucleotide sequence encoding polypeptide that comprises a targeting unit, multimerization unit and antigenic unit and one or more nucleic acid sequences encoding one or more immunostimulatory compounds, wherein the vector allows for the co-expression of the polypeptide and the one or more immunostimulatory compounds as separate molecules are disclosed by the applicant in WO 2022/238420 A2, the disclosure of which is hereby incorporated by reference.

The one or more immunostimulatory compounds can enhance the effect of the construct of the disclosure. Without wishing to be bound by the theory, the co-expression may have marked advantages on the cellular level. When a vector comprising the polynucleotide of the disclosure is administered intramuscularly to a subject, the polypeptide/multimeric protein is secreted from muscle cells and taken up by neighboring APCs. Since the immunostimulatory compound is expressed in and secreted from the same muscle cell, it can stimulate the same APC and thereby directly affect said APC, e.g. if the APC is a dendritic cell, promote the activation and maturation of it.

The polycistronic vector may be any molecule which is suitable to deliver foreign nucleic acid sequences, such as DNA or RNA, into a cell (in vitro or in vivo) where they are expressed, i.e. expression vectors.

In some embodiments, the polycistronic vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In some other embodiments, the polycistronic vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In preferred embodiments, the vector is a polycistronic DNA plasmid.

Polycistronic vectors are known in the art, hence, the skilled person is able to design and construct such polycistronic vectors.

In some embodiments, the polycistronic vector comprises one or more co-expression elements, i.e. nucleic acid sequences which allow co-expression of the polypeptide and the one or more immunostimulatory compounds as separate molecules. Co-expression elements are known in the art and are for instance disclosed in WO 2022/238420A2, the disclosure of which is hereby incorporated by reference.

In some embodiments, the polycistronic vector comprises a co-expression element which causes the polypeptide and the one or more immunostimulatory compounds to be transcribed on a single transcript but independently translated. Hence, the presence of the co-expression element results in a final production of separate translation products.

In some embodiments, such co-expression element is an IRES element (internal ribosome entry site). In other embodiments, such co-expression element is a 2A self-cleaving peptide (2A peptide), such as a T2A peptide. Such co-expression elements are known in the art.

If more than one immunostimulatory compound is expressed from the polycistronic vector, an IRES element and/or 2A peptide needs to be present in the vector, e.g. upstream of each nucleic acid sequence encoding an immunostimulatory compound.

In some other embodiments, the polycistronic vector comprises a co-expression element which causes that the polypeptide and the one or more immunostimulatory compounds are transcribed as separate transcripts, which results in separate transcription products and thus separate proteins.

In some embodiments, such co-expression element is a bidirectional promoter. In some other embodiments, such co-expression elements are various promoters, i.e. the polycistronic vector comprises a promoter for each of the nucleic acid sequences encoding either the polypeptide or the one or more immunostimulatory compounds. Both co-expression elements are known in the art.

The above-described co-expression elements can be combined in any manner, i.e. the polycistronic vector may comprise one or several of such same or different co-expression elements.

### Immunostimulatory Compounds

The polycistronic vector comprises one or more nucleic acid sequences encoding one or more immunostimulatory compounds. In some embodiments of the present disclosure, the immunostimulatory compound is a compound that stimulates APCs and the stimulation results in e.g. attraction, activation, maturation and/or proliferation of APCs.

In some embodiments, the immunostimulatory compound is one that attracts APCs, preferably one that can interact with the following surface molecules on APCs: CCR1 (C—C motif chemokine receptor 1), CCR3 (C—C motif chemokine receptor 3), CCR4 (C—C motif chemokine receptor 4), CCR5 (C—C motif chemokine receptor 5), CCR6 (C—C motif chemokine receptor 6), CCR7 (C motif chemokine receptor 7), CCR8 (C motif chemokine receptor 8) or XCR1 (X—C motif chemokine receptor 1).

In some other embodiments, the immunostimulatory compound is selected from the list consisting of CCL4, CCL5, CCL19, CCL20, CCL21, XCL1 or XCL2.

In yet some other embodiments, the immunostimulatory compound is one that promotes activation and/or maturation of APCs. In some embodiments, the immunostimulatory compound can interact with the following surface molecules on APCs: a receptor of the TNF receptor superfamily, including CD40 (cluster of differentiation 40), CD137 (4-1EE), CD27, ICOSL (CD275) or RANK.

In some embodiments, such immunostimulatory compounds may be selected from the list consisting of CD40L (CD40 ligand, CD154), CD137L (4-1 BBL, 4-1 BB ligand), CD70, ICOS (CD278) or RANKL.

In yet some other embodiments, the immunostimulatory compound is a cytokine selected from IL-2, IL-10, IL-12, TNFα and IFNγ. In yet some other embodiments, the immunostimulatory compound can be an immune signaling molecule such as MyD88 and TRIF which activate through TLR receptors.

In yet some other embodiments, the immunostimulatory compound can be a viral infection sensor such as for example RIG-1 and MDA-5.

In yet some other embodiments, the immunostimulatory compound can interact with a pattern recognition receptor on APCs, e.g. a Toll-like receptor, including TLR2, TLR4 or TLR5. In some embodiments, such immunostimulatory compound is one selected from the list consisting of pathogen-associated molecular patterns (PAMPs), such as flagellin, or protein damage-associated molecular patterns (DAMPs), such as HMGB1, HSPs (heat-shock proteins), Calrecticulin and Annexin A1. The aforementioned molecules in turn activate the following receptors on APCs: RAGE, TLR4, TLR9 and TIM-3 (for HMGB1), FPR (for Annexin A1), SREC1, LOX1 and CD91 (for HSP).

In yet some other embodiments, the immunostimulatory compound is one that promotes growth and/or expansion of APCs. In some embodiments, the immunostimulatory compound can interact with the following surface molecules on APCs: GM-CSF-receptor (granulocyte-macrophage colony-stimulating factor receptor, CD116), FLT-3R (fms like tyrosine kinase 3, CD135), IL-15R or IL-4R.

In some other embodiments, the immunostimulatory compound is a growth factor, such as GM-CSF (granulocyte-macrophage colony-stimulating factor), FLT-3L, IL-15 or IL-4.

In some embodiments, the polycistronic vector comprises nucleic acid sequences encoding 2, 3, 4, 5, 6, 7 or 8 immunostimulatory compounds, such as 2 to 6 immunostimulatory compounds, i.e., 2 or 3 or 4 or 5 or 6 different immunostimulatory compounds. The immunostimulatory compounds may be the same or different, preferably different.

In some preferred embodiments, the different immunostimulatory compounds also affect APCs differently in order to stimulate the immune system on many different levels and by that maximize the therapeutic or prophylactic effect of the construct of the disclosure.

As an example, in some embodiments, the polycistronic vector comprises nucleic acid sequences encoding 2 different immunostimulatory compounds, with the first one being an immunostimulatory compound that promotes the growth of DCs (e.g. FLT-3L) and the second one being an immunostimulatory compound that promotes activation of DCs (e.g. CD40L).

### Production of the Vector and Host Cells

The vectors disclosed herein are generally vectors suitable for transfecting a host cell for expression of the polypeptide and formation of a multimeric protein comprised of multiple of such polypeptides, if the polynucleotide comprised in the vector encodes a multimerization unit.

Thus, a further aspect of the disclosure is a host cell comprising a vector comprising a polynucleotide comprising a nucleotide sequence encoding a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the disclosure relates to a host cell comprising a vector comprising a polynucleotide comprising a nucleotide sequence encoding a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the vector is a polycistronic vector as disclosed herein.

In some embodiments, the host cell comprising the vector is a cell of a cell culture, e.g. a bacteria cell, and the polypeptide encoded by the vector is expressed in vitro. In some other embodiments, the host cell comprising the vector is a cell of a subject and the polypeptide encoded by the vector is expressed in said subject, i.e. in vivo, as a result of the administration of the vector to the subject. Suitable host cells for in vitro transfection include prokaryote cells, yeast cells, insect cells or higher eukaryotic cells. Suitable host cells for in vivo transfection are e.g., muscle cells.

In some embodiments, the vector allows for easy exchange of the various units described above, particularly the antigenic unit in case of individualized antigenic units. In some embodiments, the vector is a pUMVC4a vector or a vector comprising NTC9385R vector backbones. The antigenic unit may be exchanged with an antigenic unit cassette restricted by the SfiI restriction enzyme cassette where the 5′ site is incorporated in the nucleotide sequence encoding a GLGGL (SEQ ID NO: 51) or GLSGL (SEQ ID NO: 110) unit linker and the 3′ site is included after the stop codon in the vector.

Engineering and production methods of the vectors disclosed herein, e.g. expression vectors such as DNA and RNA plasmids or viral vectors, are well known and the skilled person will be able to design, engineer and produce such vectors using such known methods. Moreover, various commercial manufacturers offer services for vector design and production.

In one aspect, the disclosure relates to a method of producing a vector comprising a polynucleotide comprising a nucleotide sequence encoding a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers, the method comprising:
a) transfecting cells in vitro with the vector;
b) culturing said cells;
c) optionally, lysing the cells to release the vector from the cells; and
d) collecting and optionally purifying the vector.

In some embodiments, the disclosure relates to a method of producing a vector comprising a polynucleotide comprising a nucleotide sequence encoding a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers, the method comprising:
a) transfecting cells in vitro with the vector;
b) culturing said cells;
c) optionally, lysing the cells to release the vector from the cells; and
d) collecting and optionally purifying the vector.

In some embodiments, the vector is a polycistronic vector as disclosed herein.

### Polypeptides and Multimeric Proteins

In some embodiments, the constructs disclosed herein are polypeptides or multimeric proteins.

Thus, a further aspect of the disclosure is a polypeptide comprising a targeting unit selected from the group consisting of mutated human CCL3 and mutated human CCL3L1 and an antigenic unit comprising one or more antigens or parts thereof, wherein the targeting unit comprises one or more mutations, compared to the wild type, which reduce or prevent the formation of targeting unit oligomers.

A yet further aspect of the disclosure is a multimeric protein consisting of multiple polypeptides, wherein such polypeptide comprises a targeting unit selected from the group consisting of mutated human CCL3 and mutated human CCL3L1, a multimerization unit and an antigenic unit comprising one or more antigens or parts thereof, wherein the targeting unit comprises one or more mutations, compared to the wild type, which reduce or prevent the formation of targeting unit oligomers.

In some embodiments, the multimeric protein is a dimeric protein consisting of two polypeptides, wherein such polypeptide comprises a targeting unit selected from the group consisting of mutated human CCL3 and mutated human CCL3L1, a dimerization unit and an antigenic unit comprising one or more antigens or parts thereof, wherein the targeting unit comprises one or more mutations, compared to the wild type, which reduce or prevent the formation of targeting unit oligomers.

A polypeptide may be expressed in vitro, e.g. by transfecting a host cell with a vector comprising a polynucleotide which encodes the polypeptide, i.e. a vector or polynucleotide as disclosed herein. A polypeptide may further be expressed in vivo as a result of the administration of e.g. a vector (such as a DNA plasmid) comprising a polynucleotide which encodes the polypeptide (i.e. a vector as disclosed herein) to a subject, e.g. by transfection of muscle cells of said subject with the vector.

If the polynucleotide encodes a polypeptide which comprises a multimerization unit, such as a dimerization unit or trimerization unit, a multimeric protein, such as a dimeric or trimeric protein, is formed when the polypeptide is expressed.

The multimeric proteins may be homomultimers i.e., multimers formed by identical polypeptides or hetereomultimers i.e., multimers formed by different polypeptides. For example, if the multimeric protein is a dimeric protein, said dimeric protein may be a homodimer, i.e. a dimeric protein formed by two identical polypeptide molecules which comprise identical units and thus identical antigenic units. Alternatively, said dimeric protein may be a heterodimer formed by two different polypeptides, wherein e.g. polypeptide 1 and 2 comprise the same targeting units and dimerization units but each comprise different antigens in their respective antigenic units. Heteromultimeric proteins can be produced by co-transfecting cells with 2 different vectors, one that comprises a polynucleotide that encodes a polypeptide 1 and another that comprises a polynucleotide that encodes a polypeptide 2 which is different from polypeptide 1 (and isolation of the heteromultimeric protein after the polypeptides are expressed and the heteromultimeric proteins are formed, if desired).

In some embodiments, the polypeptide/multimeric protein is prepared by expression of the polypeptide in vitro.

Thus, a further aspect of the disclosure is a method for preparing a polypeptide comprising a targeting unit selected from the group consisting of mutated human CCL3 and mutated human CCL3L1 and an antigenic unit comprising one or more antigens or parts thereof, wherein the targeting unit comprises one or more mutations, compared to the wild type, which reduce or prevent the formation of targeting unit oligomers, the method comprises:
a) transfecting or transducing cells with a polynucleotide comprising a nucleotide sequence encoding the polypeptide;
b) culturing the cells;
c) isolating the polypeptide from the cells; and
d) optionally purifying the isolated polypeptide.

In some embodiments, said polynucleotide is comprised in the vector or polycistronic vector as disclosed herein.

A further aspect of the disclosure is a method for preparing a multimeric protein consisting of multiple polypeptides, such polypeptide comprising a targeting unit selected from the group consisting of mutated human CCL3 and mutated human CCL3L1 and an antigenic unit comprising one or more antigens or parts thereof, wherein the targeting unit comprises one or more mutations, compared to the wild type, which reduce or prevent the formation of targeting unit oligomers, the method comprises:
a) transfecting or transducing cells with a polynucleotide comprising a nucleotide sequence encoding the polypeptide;
b) culturing the cells;
c) isolating the multimeric protein from the cells; and
d) optionally purifying the isolated multimeric protein.

In some embodiments, said polynucleotide is comprised in the vector or polycistronic vector as disclosed herein.

Isolation of the polypeptide/multimeric protein and the optional purification can be carried out by methods known in the art, including precipitation, differential solubilization and chromatography.

### Pharmaceutical Compositions

In some embodiments of the present disclosure, the constructs (i.e. polynucleotides, polypeptides/multimeric proteins) and vectors disclosed herein are for use as a medicament.

Thus, in some embodiments of the present disclosure, the construct or vector is provided in a pharmaceutical composition comprising the construct or vector and a pharmaceutically acceptable carrier.

Thus, in one aspect, the disclosure relates to a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers.

Alternatively, in one aspect, the disclosure relates to a pharmaceutical composition comprising (a) a pharmaceutically acceptable carrier and (b) (i) a polypeptide comprising a targeting unit selected from the group consisting of mutated human CCL3 and mutated human CCL3L1, and an antigenic unit comprising one or more antigens or parts thereof, wherein the targeting unit comprises one or more mutations, compared to the wild type, which reduce or prevent the formation of targeting unit oligomers; or (ii) a polynucleotide encoding the polypeptide as defined in (i).

In some embodiments, the disclosure relates to a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, wherein the targeting unit comprises one or more mutations, compared to the wild type, which reduce or prevent the formation of targeting unit oligomers; or (ii) a polynucleotide encoding the polypeptide as defined in (i); or (iii) a multimeric protein consisting of multiple polypeptides as defined in (i).

In another aspect, the disclosure relates to a pharmaceutical composition comprising (i) a pharmaceutically acceptable carrier and (ii) a vector comprising a polynucleotide comprising a nucleotide sequence encoding a targeting unit selected from the group consisting of mutated human CCL3 and mutated human CCL3L1 and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the disclosure relates to a pharmaceutical composition comprising (i) a pharmaceutically acceptable carrier and (ii) a vector comprising a polynucleotide comprising a nucleotide sequence encoding a targeting unit selected from the group consisting of mutated human CCL3 and mutated human CCL3L1, a multimerization unit and an antigenic unit comprising one or more antigens or parts thereof, wherein the amino acid sequence of the targeting unit comprises one or more mutations, compared to the amino acid sequence of the wild type, which reduce or prevent the formation of targeting unit oligomers.

In some embodiments, the one or more antigens or parts thereof are disease-relevant antigens or parts thereof. In some embodiments, the vector is a polycistronic vector as disclosed herein.

Suitable pharmaceutically acceptable carriers include, but are not limited to, saline, buffered saline, such as PBS, dextrose, water, glycerol, ethanol, aqueous buffers, such as isotonic aqueous buffers or Tyrode's buffer, and combinations thereof.

In some embodiments, the pharmaceutically acceptable carrier is an aqueous buffer. In some other embodiments, the aqueous buffer is Tyrode's buffer, e.g. Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose.

In some embodiments, the pharmaceutical composition comprises a polynucleotide or vector and further comprises molecules that facilitate the transfection of cells with the polynucleotide or vector as disclosed herein, e.g. facilitate the transfection of muscle cells of a subject, i.e. one or more transfection agents.

Transfection agents for polynucleotides are known in the art and include positively charged molecules that interact with negatively charged molecules like DNA or RNA and form a positively charged transfection agent-DNA or transfection agent-RNA complex. Such complexes can interact with negatively charged cell membranes which enables the uptake of the complexes and thus the delivery of the DNA or RNA into the cell.

In some specific embodiments pharmaceutical composition comprises a pharmaceutically acceptable amphiphilic block co-polymer comprising blocks of poly(ethylene oxide) and polypropylene oxide.

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the pharmaceutical composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In some other embodiments, the pharmaceutical composition comprises the amphiphilic block co-polymer in an amount of from 2% w/v to 5% w/v, such as about 3% w/v.

In some embodiments, the pharmaceutical composition comprises a polypeptide or multimeric protein and further comprises an adjuvant. Adjuvants for polypeptide/protein comprising pharmaceutical compositions are known in the art and include but are not limited to poly-ICLC, 1018 ISS, aluminum salts, Amplivax, AS 15, BCG, CP-870,893, CpG7909, CyaA, dSLIM, GM-CSF, IC30, IC31, Imiquimod, ImuFact EV1 P321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PLGA microparticles, resiquimod, SRL172, virosomes and other virus-like particles, YF-17D, VEGF trap, R848, beta-glucan, Pam3Cys, Aquila's QS21 stimulon, vadimezan, and/or AsA404 (DMXAA). However, due to the presence of the targeting unit, the pharmaceutical composition can be administered without additional adjuvant. Thus, in some embodiments, the pharmaceutical composition does not comprise an adjuvant.

The pharmaceutical composition may be formulated in any way suitable for administration to a subject, e.g. such as a liquid formulation for injection, e.g. for intradermal or intramuscular injection.

The pharmaceutical composition may be administered in any way suitable for administration to a subject, such as administered by intradermal, intramuscular, or subcutaneous injection, or by mucosal or epithelial application, such as intranasal or oral.

In a preferred embodiment, the pharmaceutical composition is administered by intramuscular or intradermal injection.

The amount of vector, e.g. DNA plasmid, in the pharmaceutical composition may vary depending on whether the pharmaceutical composition is administered for prophylactic or therapeutic treatment.

The pharmaceutical composition of the disclosure typically comprises the polynucleotide, e.g. comprised in a vector, e.g. DNA plasmid, in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The pharmaceutical composition of the disclosure typically comprises the polypeptide/multimeric protein in a range of from 5 μg to 5 mg.

The amount of polynucleotide, vector, polypeptide/multimeric protein may vary depending on whether the pharmaceutical composition is administered for prophylactic or therapeutic treatment.

In a preferred embodiment, the pharmaceutical composition is a sterile pharmaceutical composition.

The pharmaceutical composition may be prepared by dissolving the polynucleotide, vector, polypeptide or multimeric protein in the pharmaceutically acceptable carrier and optionally adding other compounds which may be present in the pharmaceutical composition, such as transfection agents or adjuvants.

### Treatment

In some aspects of the present disclosure, the polynucleotide, vector, polypeptide/multimeric protein is for use in the therapeutic or prophylactic treatment of a disorder, such as a disorder in humans.

Thus, in one aspect, the disclosure relates to a method of treating a subject having a disease or being in need of prevention of said disease, the method comprising administering to the subject a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof which are relevant for said disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers.

Alternatively, in one aspect, the disclosure relates to a method of treating a subject having a disease or being in need of prevention of said disease, the method comprising administering to the subject a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof which are relevant for said disease, wherein the targeting unit comprises one or more mutations, compared to the wild type, which reduce or prevent the formation of targeting unit oligomers; or (ii) a polynucleotide encoding the polypeptide as defined in (i).

In some embodiments, the disclosure relates to a method of treating a subject having a disease or being in need of prevention of said disease, the method comprising administering to the subject a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for said disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a method of treating a subject having a disease or being in need of prevention of said disease, the method comprising administering to the subject a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof which are relevant for said disease, wherein the targeting unit comprises one or more mutations, compared to the wild type, which reduce or prevent the formation of targeting unit oligomers; or (ii) a polynucleotide encoding the polypeptide as defined in (i); or a multimeric protein consisting of multiple polypeptides as defined in (i).

Also disclosed herein is a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers, for use in the treatment of a subject having said disease or being in need of prevention of said disease.

Alternatively, also disclosed herein is a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i), for use in the treatment of a subject having said disease or being in need of prevention of said disease.

In some embodiments, the disclosure relates to a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers for use in the treatment of a subject having said disease or being in need of prevention of said disease.

Alternatively, in some embodiments, the disclosure relates to a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or a multimeric protein consisting of multiple polypeptides as defined in (i), for use in the treatment of a subject having said disease or being in need of prevention of said disease.

In some embodiments, said pharmaceutical composition is administered to said subject.

Also disclosed herein is the use of (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers for the manufacture of a medicament for use in the treatment of a subject having said disease or being in need of prevention of said disease.

Alternatively, also disclosed herein is the use of (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i), for the manufacture of a medicament for use in the treatment of a subject having said disease or being in need of prevention of said disease.

In some embodiments, the disclosure relates to the use of (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers for the manufacture of a medicament for use in the treatment of a subject having said disease or being in need of prevention of said disease.

Alternatively, in some embodiments, the disclosure relates to the use of (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or a multimeric protein consisting of multiple polypeptides as defined in (i), for the manufacture of a medicament for use in the treatment of a subject having said disease or being in need of prevention of said disease.

In some embodiments, said medicament is a pharmaceutical composition comprising (i), (ii) or (iii) and a pharmaceutically acceptable carrier and/or said medicament is administered to said subject.

Also disclosed herein is the use of a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers for the manufacture of a medicament for use in the treatment of a subject having said disease or being in need of prevention of said disease.

Alternatively, also disclosed herein is the use of a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i), for the manufacture of a medicament for use in the treatment of a subject having said disease or being in need of prevention of said disease.

In some embodiments, the disclosure relates to the use of a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers for the manufacture of a medicament for use in the treatment of a subject having said disease or being in need of prevention of said disease.

Alternatively, in some embodiments, the disclosure relates to the use of a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or a multimeric protein consisting of multiple polypeptides as defined in (i), for the manufacture of a medicament for use in the treatment of a subject having said disease or being in need of prevention of said disease.

In some embodiments, said medicament is administered to said subject.

Also disclosed herein is the use of a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers, for treating a subject having said disease or being in need of prevention of said disease.

Alternatively, also disclosed herein is the use of a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i), for treating a subject having said disease or being in need of prevention of said disease.

In some embodiments, the disclosure relates to the use of a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers, for treating a subject having said disease or being in need of prevention of said disease.

Alternatively, in some embodiments, the disclosure relates to the use of a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or (iii) a multimeric protein consisting of multiple polypeptides as defined in (i), for treating a subject having said disease or being in need of prevention of said disease.

In some embodiments, said use comprises administering said pharmaceutical composition to said subject.

Also disclosed herein is a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers, when used in the therapeutic or prophylactic treatment of said disease.

Alternatively, also disclosed herein is a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i), when used in the therapeutic or prophylactic treatment of said disease.

In some embodiments, the disclosure relates to a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers, when used in the therapeutic or prophylactic treatment of said disease.

Alternatively, in some embodiments, the disclosure relates to a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or (iii) a multimeric protein consisting of multiple polypeptides as defined in (i), when used in the therapeutic or prophylactic treatment of said disease.

In some embodiments, said pharmaceutical composition is administered to a subject in need of such therapeutic or prophylactic treatment.

Also disclosed herein is a medicament for the treatment or prevention of a disease in a subject having said disease or being in need of prevention of said disease by administering to the subject a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1 and an antigenic unit comprising one or more antigens or parts thereof, which are relevant for a disease; or (ii) a polypeptide encoded by the nucleotide sequence as defined in (i); wherein the amino acid sequence of the targeting unit comprises one or more mutations, compared to the amino acid sequence of the wild type, which reduce or prevent the formation of targeting unit oligomers.

Alternatively, also disclosed herein is a medicament for the treatment or prevention of a disease in a subject having said disease or being in need of prevention of said disease by administering to the subject a pharmaceutical composition comprising (a) a pharmaceutically acceptable carrier and (b) (i) a polypeptide comprising a targeting unit selected from the group consisting of mutated human CCL3 and mutated human CCL3L1 and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i).

In some embodiments, the disclosure relates to a medicament for the treatment or prevention of a disease in a subject having said disease or being in need of prevention of said disease by administering to the subject a pharmaceutical composition comprising (a) a pharmaceutically acceptable carrier and (b) (i) a polynucleotide comprising a nucleotide sequence encoding a targeting unit selected from the group consisting of mutated human CCL3 and mutated human CCL3L1, a multimerization unit and an antigenic unit comprising one or more antigens or parts thereof, which are relevant for a disease; or (ii) a polypeptide encoded by the nucleotide sequence as defined in (i); or (iii) a multimeric protein consisting of multiple polypeptides as defined in (ii); wherein the amino acid sequence of the targeting unit comprises one or more mutations, compared to the amino acid sequence of the wild type, which reduce or prevent the formation of targeting unit oligomers.

Alternatively, in some embodiments, the disclosure relates to a medicament for the treatment or prevention of a disease in a subject having said disease or being in need of prevention of said disease by administering to the subject a pharmaceutical composition comprising (a) a pharmaceutically acceptable carrier and (b) (i) a polypeptide comprising a targeting unit selected from the group consisting of mutated human CCL3 and mutated human CCL3L1, a multimerization unit and an antigenic unit comprising one or more antigens or parts thereof, which are relevant for a disease, wherein the targeting unit comprises one or more mutations, compared to the wild type, which reduce or prevent the formation of targeting unit oligomers; or (ii) a polynucleotide encoding the polypeptide as defined in (i); or a multimeric protein consisting of multiple polypeptides as defined in (i).

In some embodiments of the above-disclosed methods and uses, the pharmaceutical composition comprises the polynucleotide in a vector, e.g. a vector as disclosed herein.

In the method of treatment/use of the pharmaceutical composition or medicament or construct disclosed herein, the pharmaceutical composition/medicament/construct is preferably administered in a therapeutically effective or prophylactically effective amount. Such an amount may be administered in one administration, i.e. one dose, or in several administrations, i.e. repetitive doses, i.e. in a series of doses, e.g. over the course of several days, weeks or months.

The actual dose to be administered may vary and depend on whether the treatment is a prophylactic or therapeutic treatment, the age, weight, gender, medical history, pre-existing conditions and general condition of the subject, the severity of the disease being treated and the judgment of the health care professionals.

In the method of treatment/use of the pharmaceutical composition or medicament or construct disclosed herein, the pharmaceutical composition/medicament/construct may be administered in way as described herein, e.g. described in the section “Pharmaceutical compositions”.

The method of treatment/use of the pharmaceutical composition or medicament or construct disclosed herein can be continued for as long as the clinician overseeing the patient's care deems the method to be effective and the treatment to be needed.

In some embodiments, the method of treatment/use of the pharmaceutical composition or medicament or construct disclosed herein is for treating cancer and the pharmaceutical compositions/medicaments comprise a construct for use in the treatment of a cancer, i.e. a construct comprising an antigenic unit comprising one or more cancer antigens or parts thereof. Such antigenic units, both for individualized and non-individualized constructs, and various embodiments thereof, have been described in detail herein.

The cancer may be a solid or a liquid cancer. Examples of solid cancers are cancers forming a solid mass, e.g. a tumor. Examples of liquid cancers are cancers present in body fluid, such as lymphomas or blood cancers.

In some embodiments of the present disclosure, the method of treatment/use of the pharmaceutical composition or medicament or construct disclosed herein is one of treating a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

In some embodiments, the method of treatment/use of the pharmaceutical composition or medicament or construct disclosed herein is for treating an infectious disease and the pharmaceutical composition/medicament comprise a construct for use in the treatment of an infectious disease, i.e. a construct comprising an antigenic unit comprising one or more infectious antigens or parts thereof. Such antigenic units and various embodiments thereof, have been described in detail herein.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

For Examples 1-6, all nucleotide sequences were ordered from Genscript Biotech B., Netherlands and cloned into the expression vector (DNA plasmid) pUMVC4a. In all the Examples, the SEQ ID NOs refer to the amino acid sequences of polypeptides according to the disclosure (or elements/units comprised therein) encoded by said nucleotide sequences.

### Example 1

All DNA plasmids designed, produced and tested in Examples 1-3 comprise nucleic acid sequences encoding the elements/units listed in Table 1. DNA plasmids VB1020 and TECH011-CV0006 of this Example 1 further comprise nucleic acid sequences encoding the units listed in Table 2.

TABLE 1

| Elements/Units | Description and sequence |
|---|---|
| Signal peptide | Natural leader sequence for hCCL3L1 |
| | SEQ ID NO: 117 |
| Dimerization unit | Hinge-region 1 from human IgG |
| | Amino acids 1-12 of SEQ ID NO: 18 |
| | Hinge-region 4 from human IgG3 |
| | Amino acids 13-27 of SEQ ID NO: 18 |
| | Glycine-serine-linker |
| | Amino acids 28-37 of SEQ ID NO: 18 |
| | CH3 domain from human IgG3 |
| | Amino acids 38-144 of SEQ ID NO: 18 |
| Unit linker | Glycine-leucine linker |
| | SEQ ID NO: 51 |

TABLE 2

| | VB1020 | TECH011-CV006 |
|---|---|---|
| Element/Units | SEQ ID NO: 118 | SEQ ID NO: 119 |
| Targeting unit | hCCL3L1 wild type | hCCL3L1 with D27A mutation |
| | SEQ ID NO: 2 | SEQ ID NO: 5 |

| | |
|---|---|
| Antigenic unit | HPV16 E7 antigen and HPV16 E6 antigen |
| | separated by a glycine-serine linker |
| | SEQ ID NO: 120 |

DNA plasmids VB1020 and TECH011-CV006 comprise nucleic acid sequences encoding a protein comprising a hCCL3L1 targeting unit (wild type or comprising a D27A mutation), dimerization unit and an antigenic unit comprising human papilloma virus 16 (HPV16) antigens E7 and E6.

TECH011-CV006 is a DNA plasmid comprising an embodiment of construct as disclosed herein, i.e. a non-individualized construct for use in the treatment of cancer, comprising an antigenic unit comprising several shared cancer antigens, with the shared cancer antigens being viral shared cancer antigens (here: antigens from HPV16 which causes for certain types of cancer). It is however also a DNA plasmid comprising an embodiment of a construct as disclosed herein, i.e. a construct for use in the treatment of infectious diseases, comprising comprises an antigenic unit comprising antigens derived from a pathogen (here: antigens derived from HPV16). VB1020 serves as a control comprising the identical nucleic acid sequence as TECH011-CV006, but for the D27A mutation in the sequence of the hCCL3L1 targeting unit.

### A. Assessment of Expression and Secretion of Proteins Encoded by VB1020 and TECH011-CV006 DNA Plasmids

Briefly, Expi293F cells (2×10⁶ cells/mL, 1 mL) were seeded in a 96-well culture plate. The cells were transfected with 0.64 μg/mL plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37 °C). The plates were incubated for 3 days before the supernatant was harvested.

The proteins present in the supernatants expressed from the DNA plasmids were characterized in a sandwich ELISA using mouse anti-human IgG CH3 domain antibody (capture antibody, 100 μL/well, 1 μg/mL, MCA878G, Bio-Rad) and goat anti-human MIP-1a antibody (biotinylated detection antibody, 100 μL/well, 0.2 μg/mL, BAF270, R&D systems). Supernatants collected from cells transfected with VB1020 were diluted 1:50 and from cells transfected with TECH011-CV006 were diluted 1:1500, respectively).

Protein concentration in the cell supernatant was interpolated from a standard curve of purified protein (protein comprising the hCCL3L1 targeting unit (SEQ ID NO: 2) and the dimerization unit listed in Table 1 with a C-terminal EPEA tag). A sample of ExpiFectamine 293 reagent treated cells (transfection control) was included as a negative control.

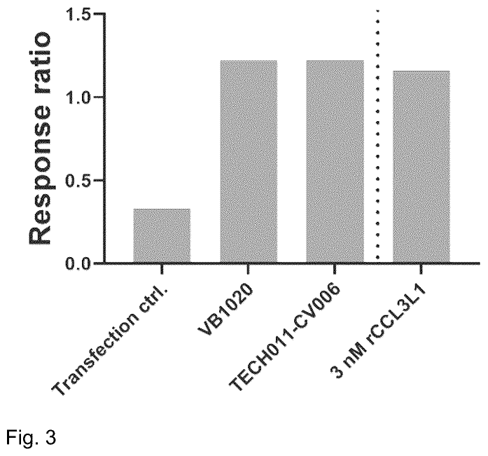

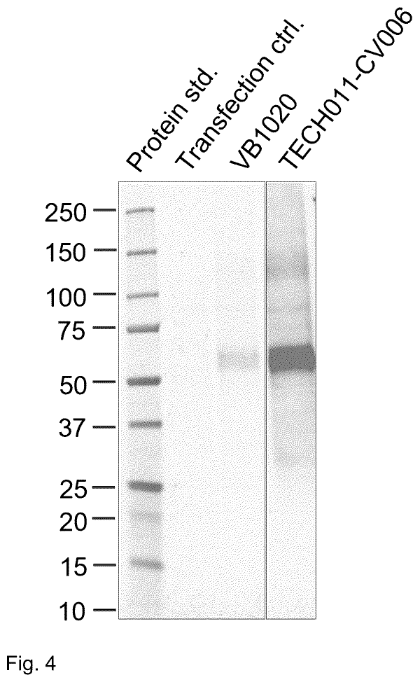

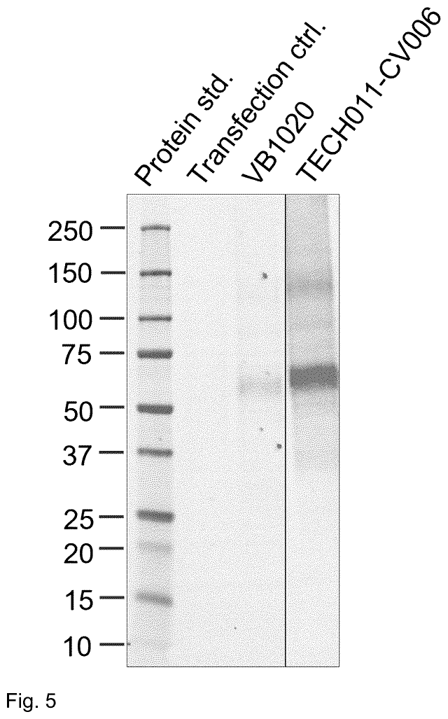

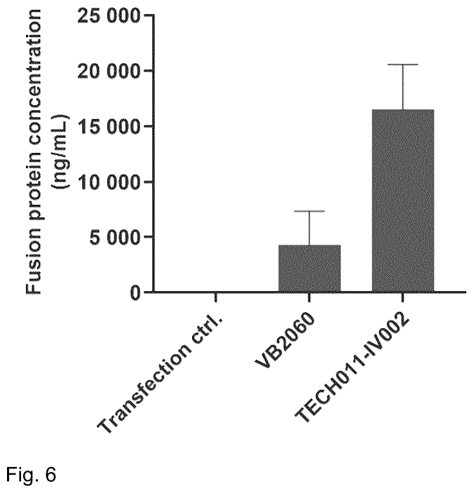

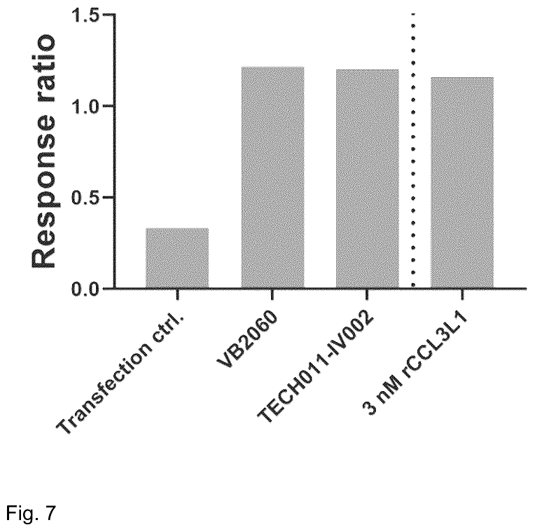

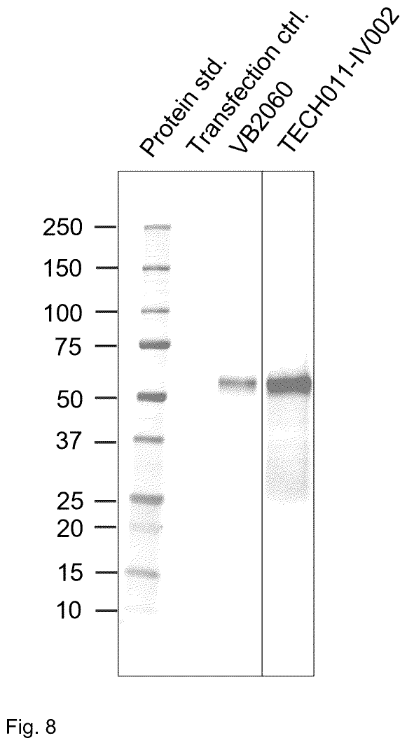

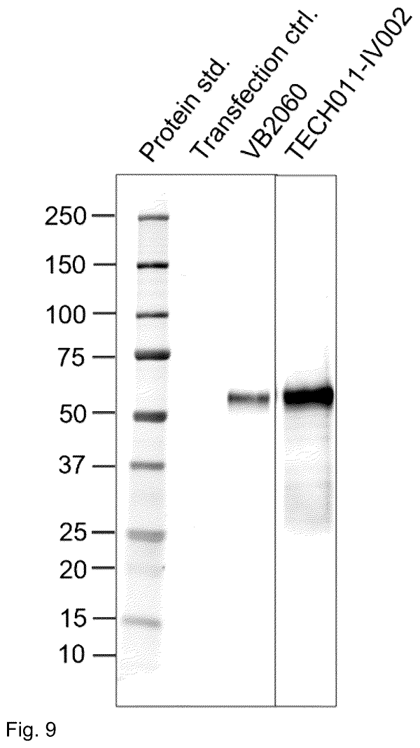

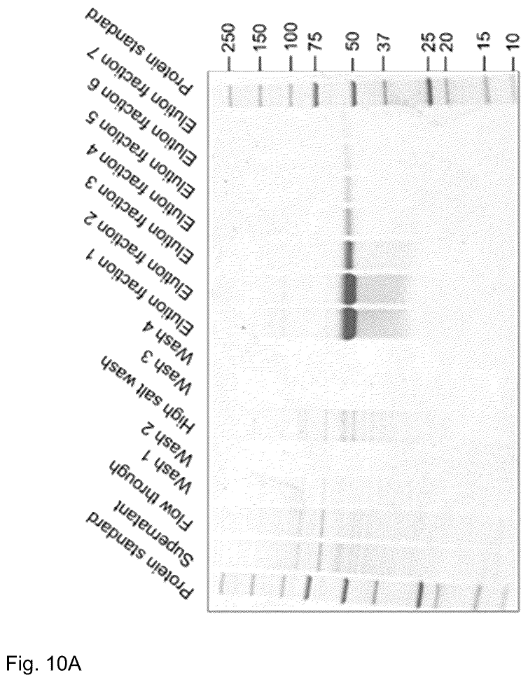

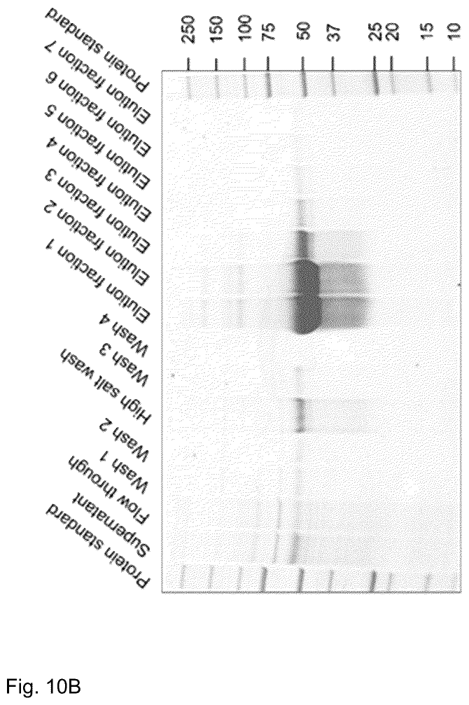

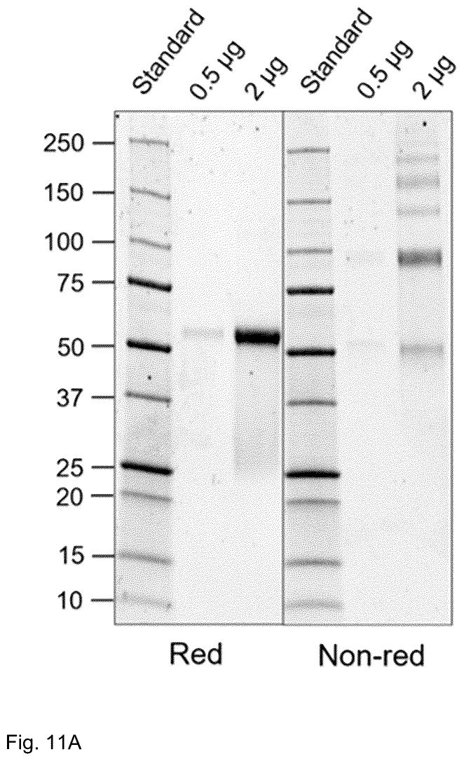

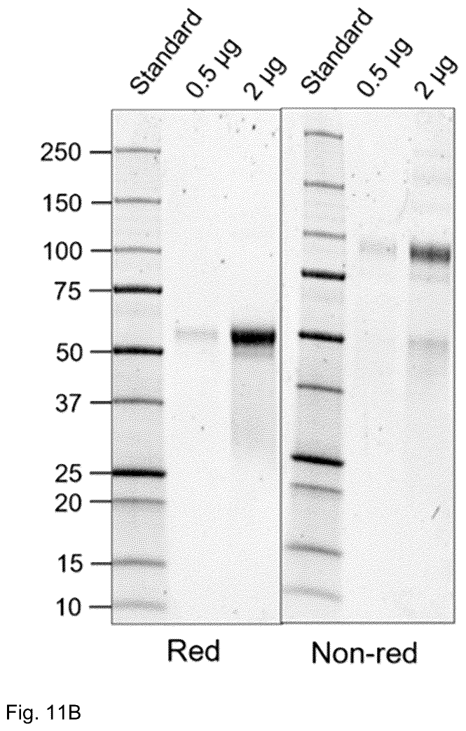

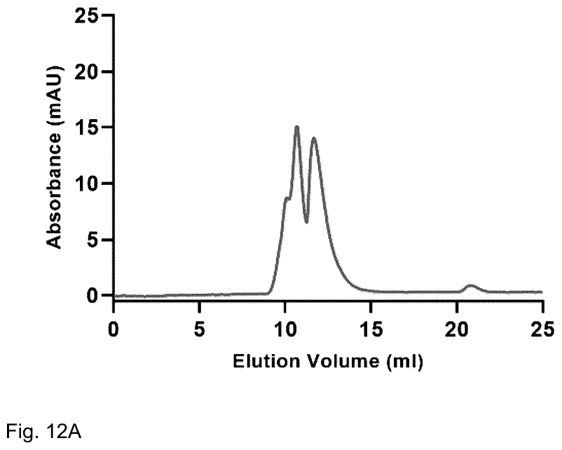

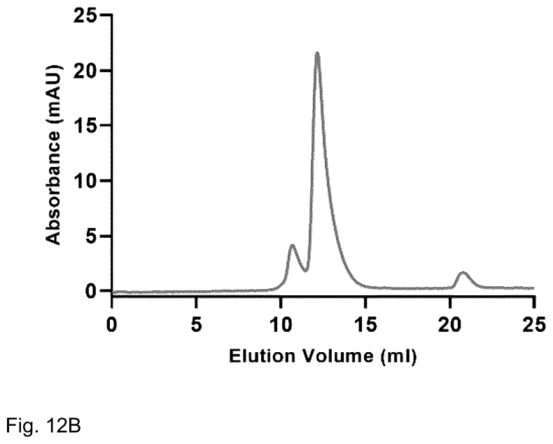

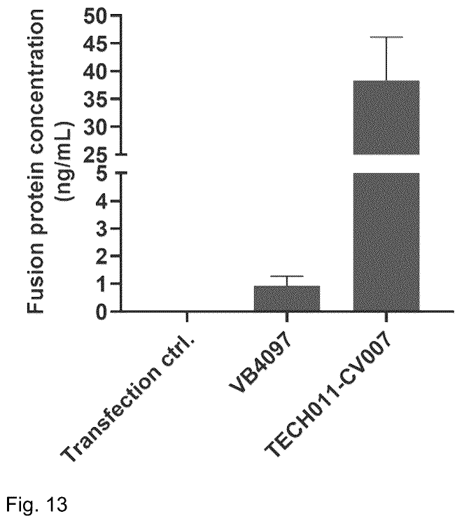

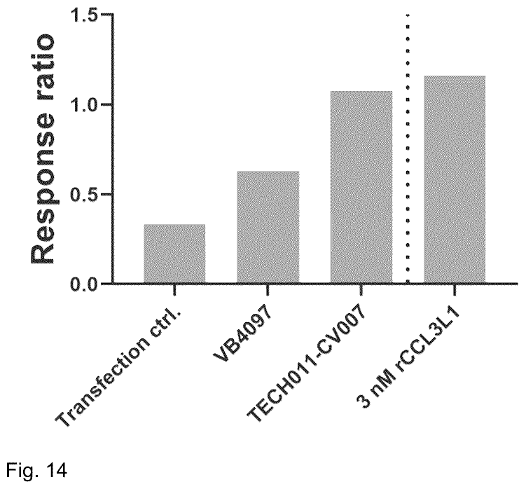

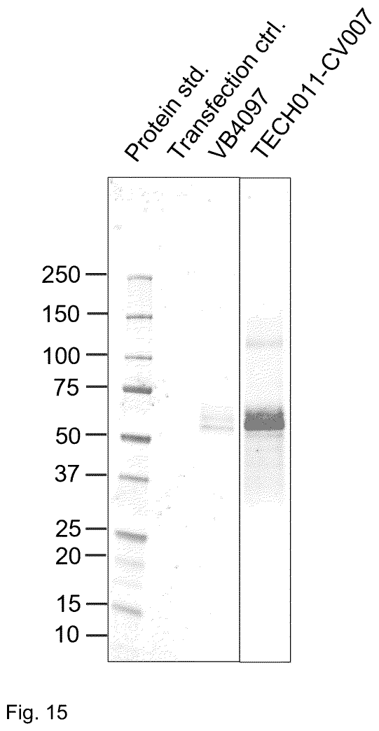

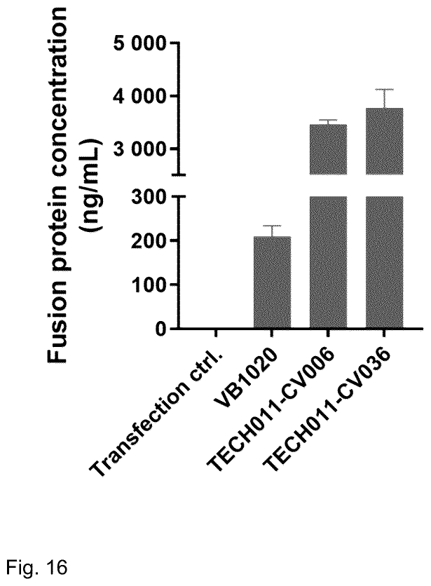

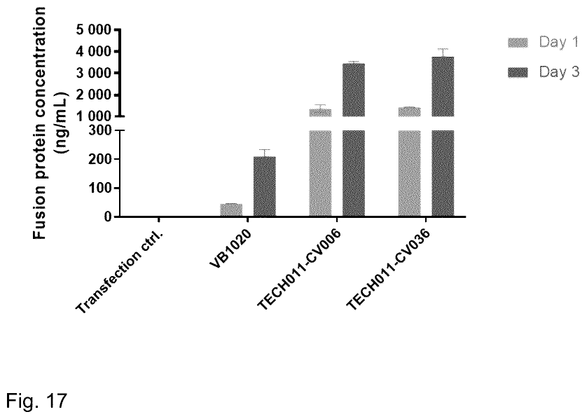

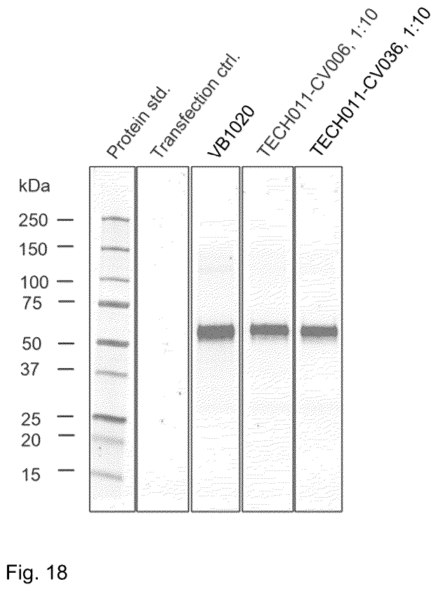

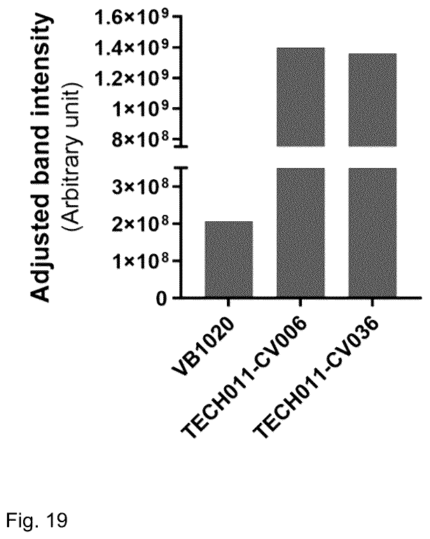

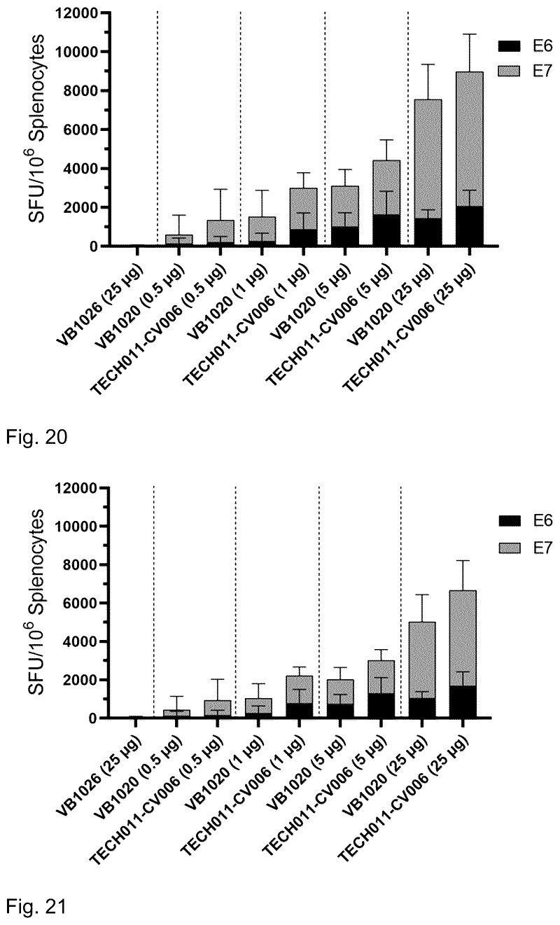

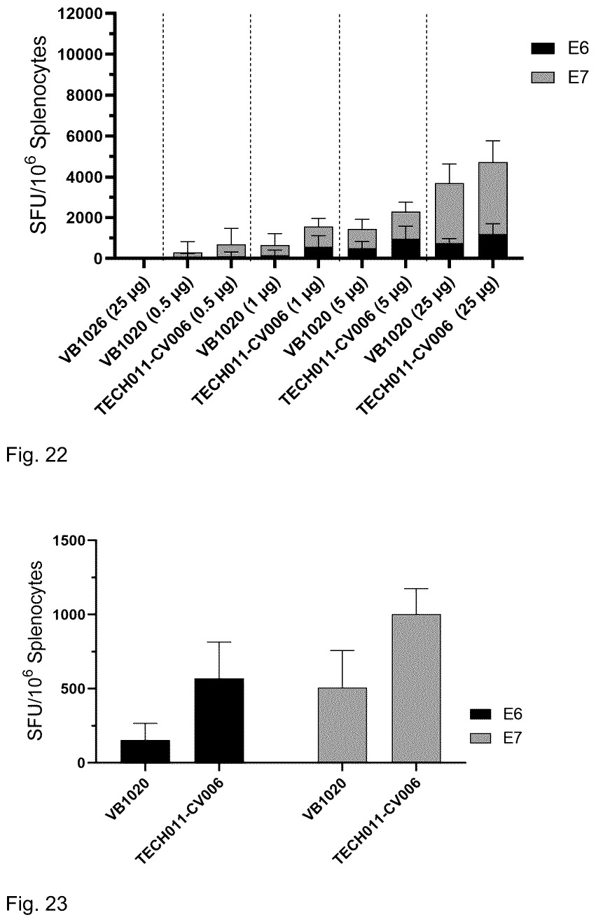

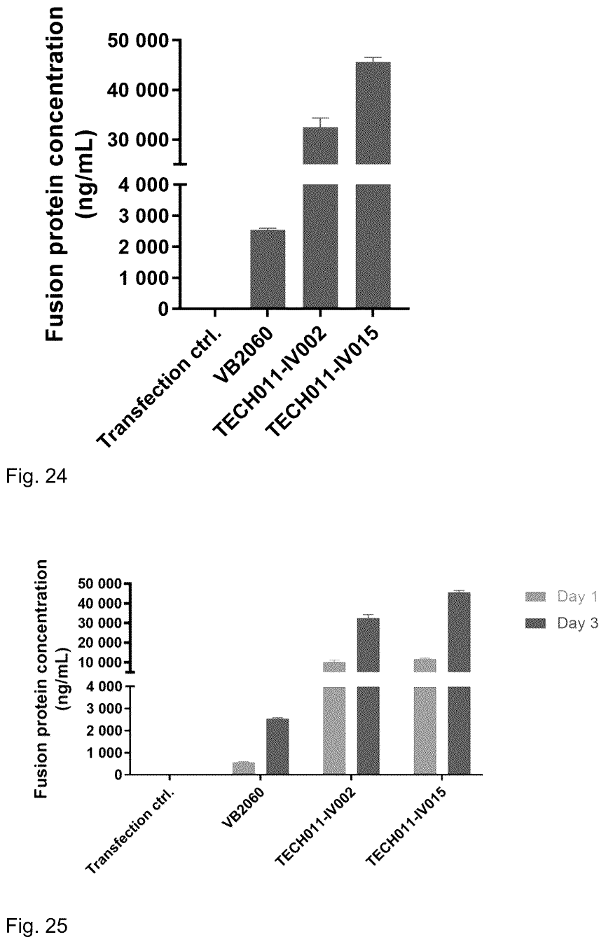

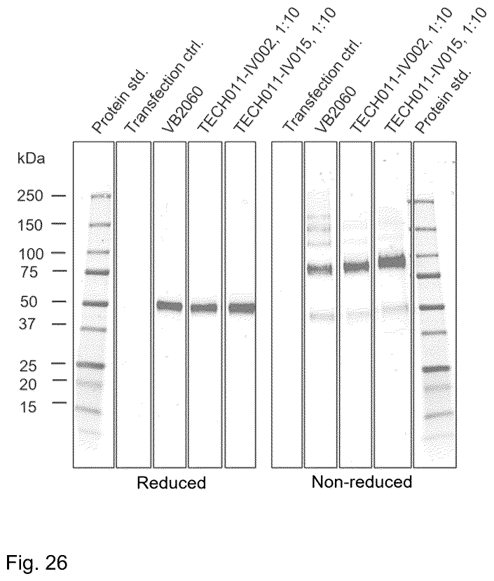

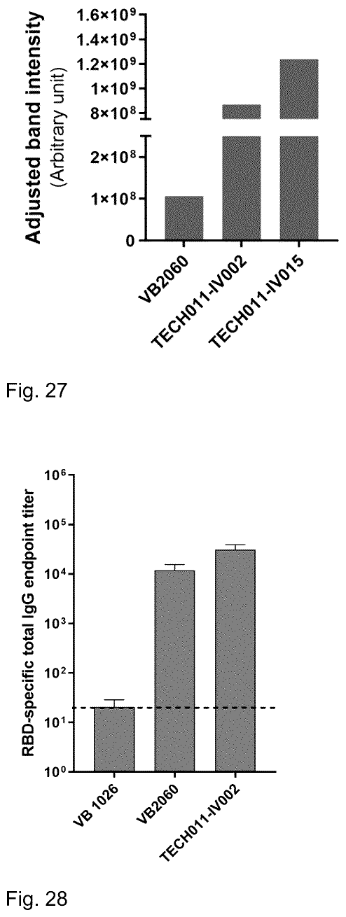

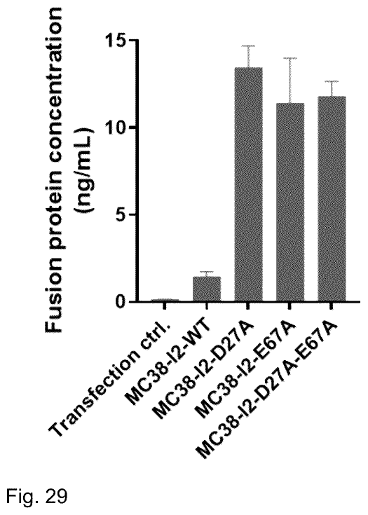

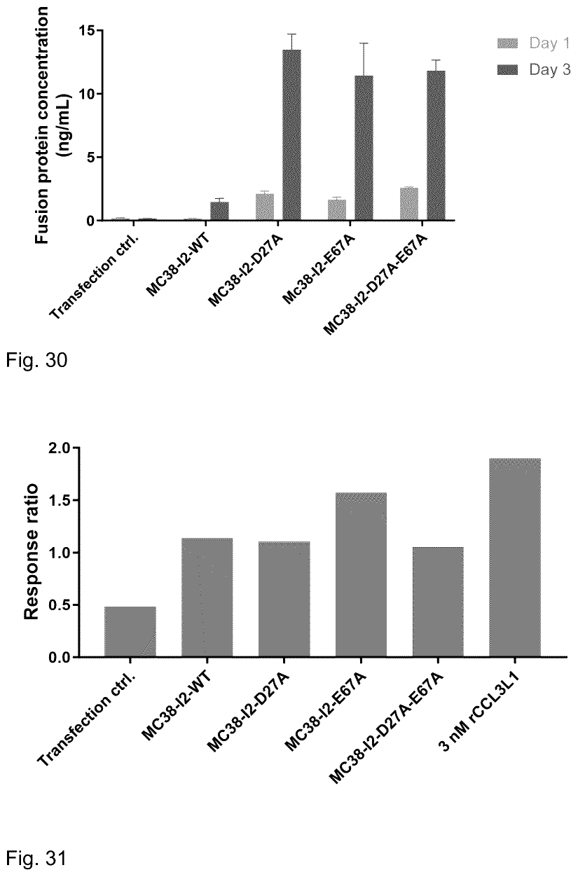

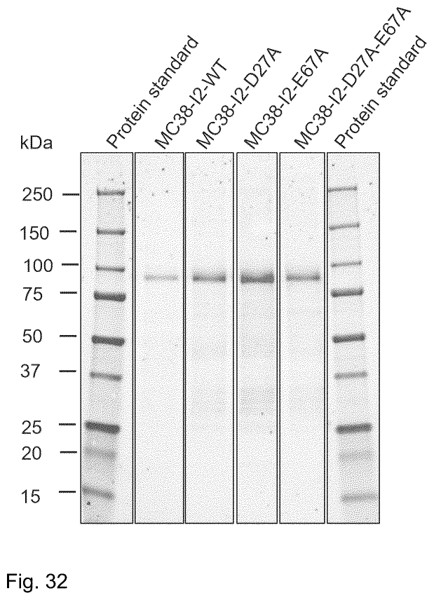

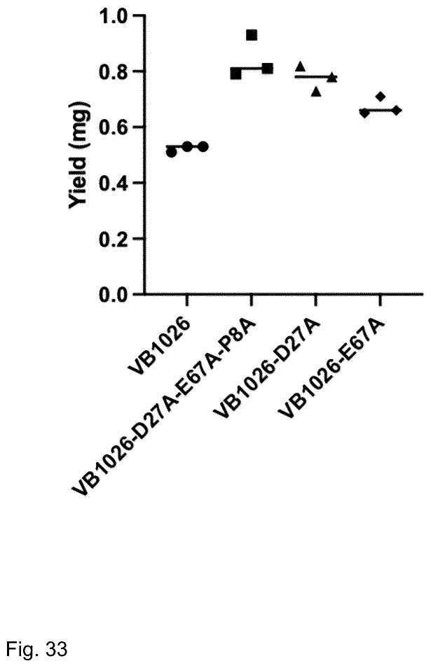

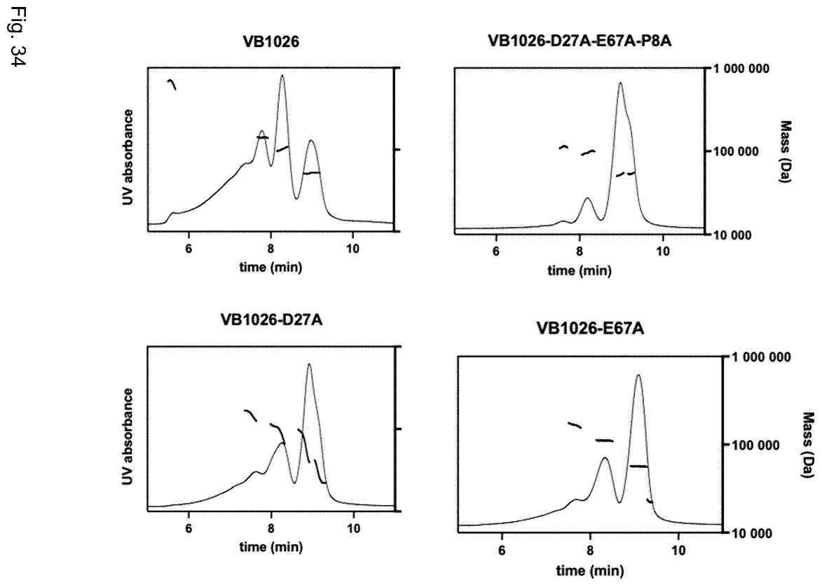

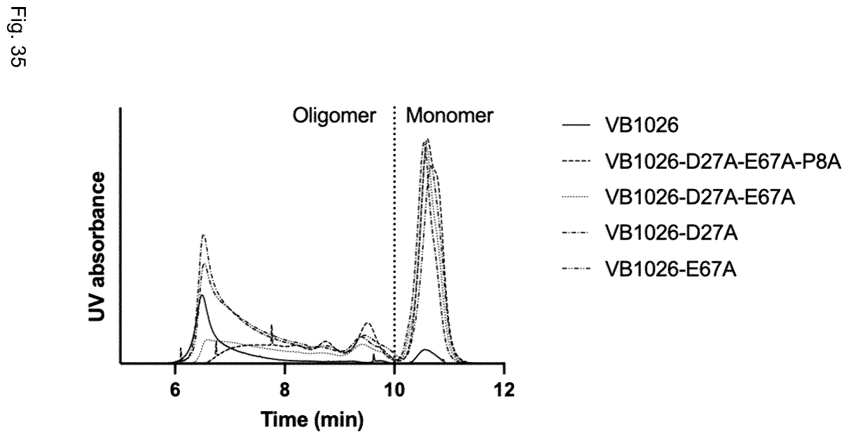

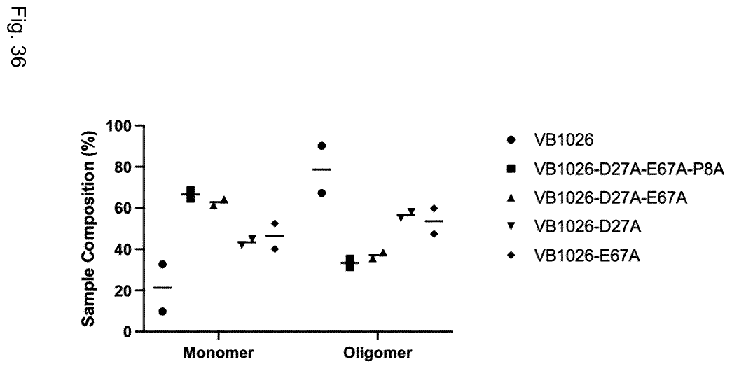

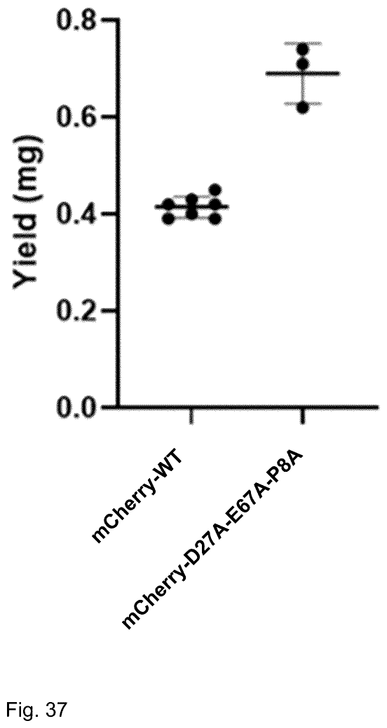

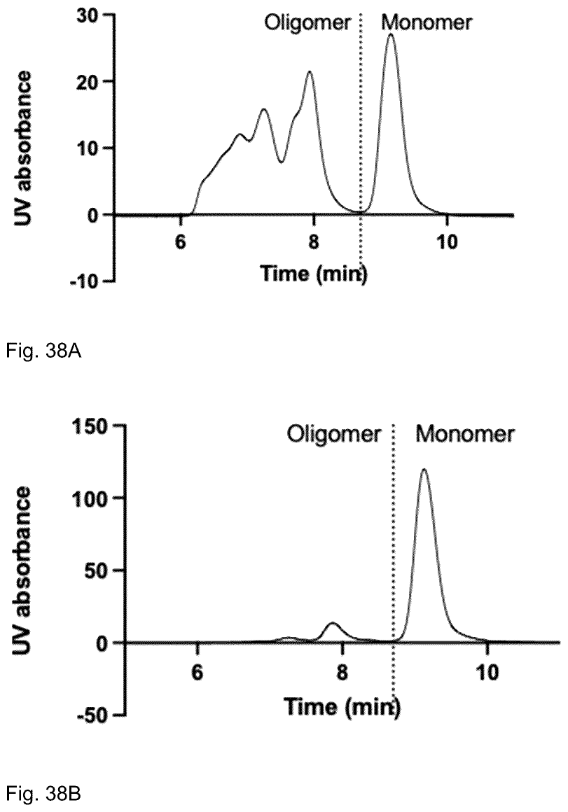

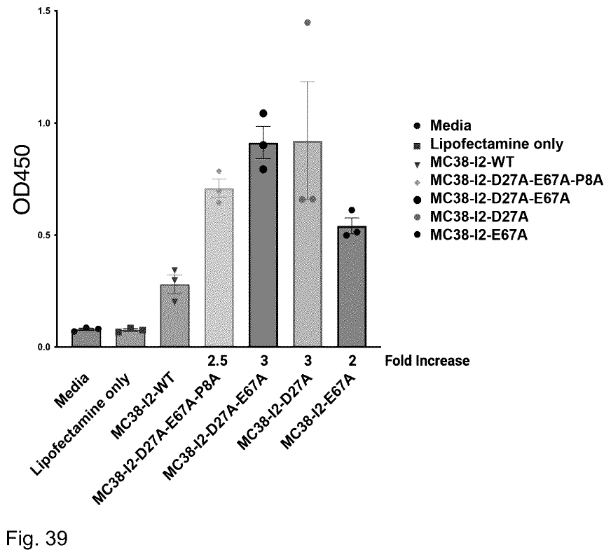

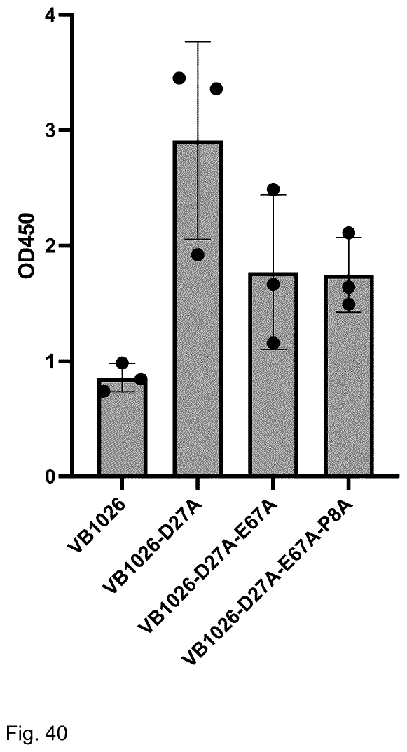

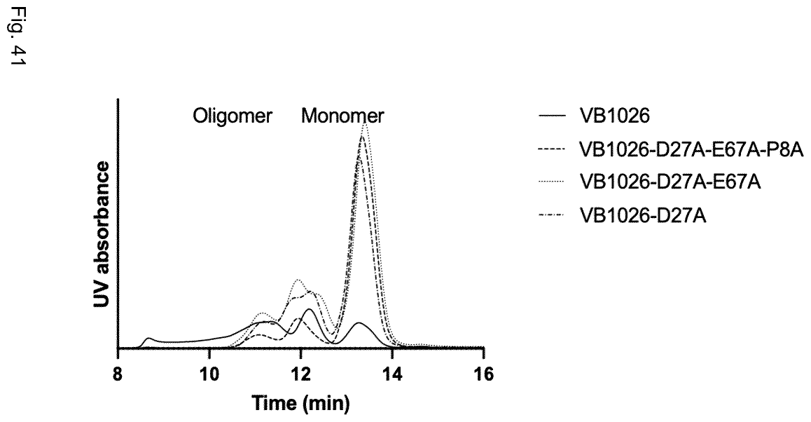

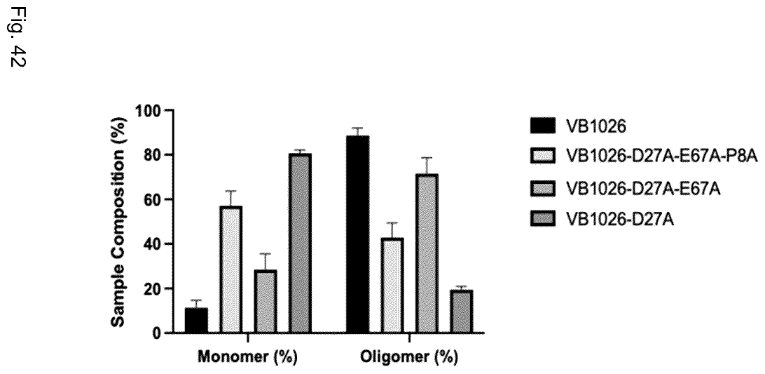

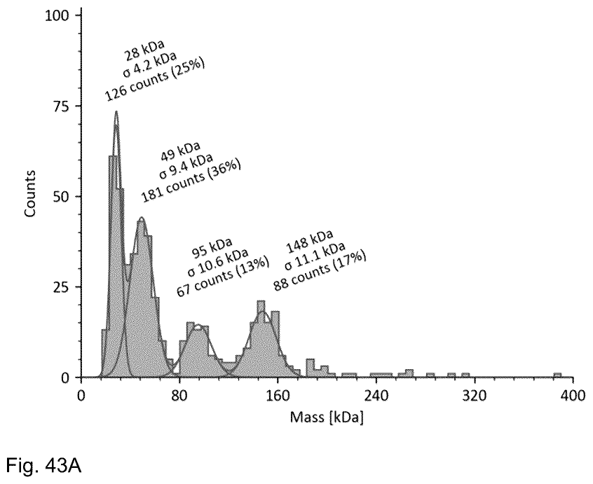

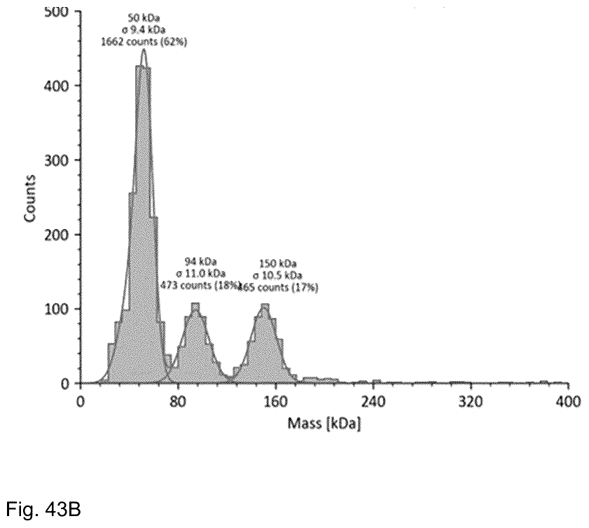

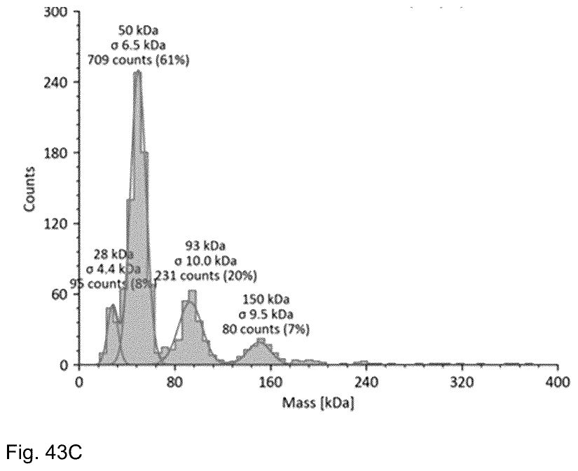

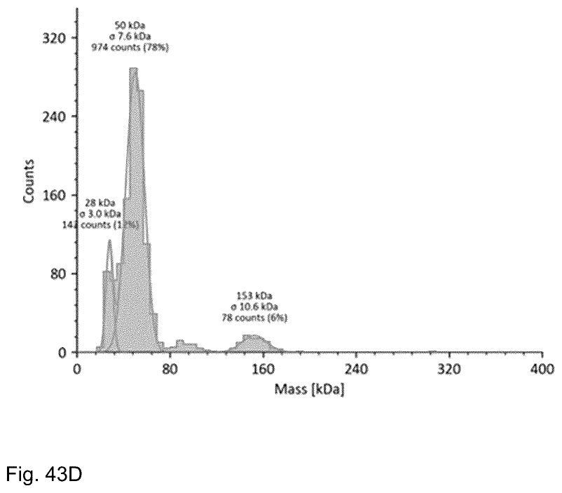

The results presented in FIG. 2 demonstrate that the proteins encoded by VB1020 and TECH011-CV006 were well expressed and secreted from transfected Expi293F cells. The data further show that secretion of the protein comprising a D27A mutated hCCL3L1 targeting unit which was expressed from TECH011-CV006 is markedly increased, compared to secretion of the protein comprising the wild type hCCL1 L3 targeting unit.

### B. Assessment of Receptor Activation by Proteins Encoded by VB1020 and TECH011-CV006 DNA Plasmids

The hCCR5 reporter assay was performed with cell supernatant of Example 1A from cells transfected with VB1020 and TECH011-CV006, respectively, using a commercially available kit (Thermo Fisher Scientific, K1788) according to the manufacturer's protocol. Briefly, the CCR5-bla U2OS cells express functional cell membrane-bound hCCR5 receptors, where the intracellular part of the receptor is fused to a β-lactamase transcription factor. Upon hCCL3L1 binding, β-arrestin proteins are recruited to the activated receptor. A protease which is fused to β-arrestin will then cleave off the transcription factor bound to hCCR5, leading to transcription of β-lactamase. To measure the β-lactamase (bla) activity, a fluorescent bla substrate is added to the cells. Once added to the cells, the lipophilic bla substrate (CCF4-AM) is cleaved and converted by endogenous cytoplasmic esterases into its negatively charged form (CCF4) and is retained in the cytosol. CCF4 contains two fluorophores: coumarin and fluorescein. Upon excitation at 409 nm in the absence of bla (no activation of CCR5), a fluorescence energy transfer (FRET) from coumarin to fluorescein will occur in the CCF4 substrate, which can then be detected at 530 nm (green, fluorescent light) due to excitation of fluorescein. Upon excitation at 409 nm in the presence of bla (activation of CCR5), CCF4 will be cleaved, causing disruption of the FRET and excitation of coumarin, which can be detected at 460 nm (blue, fluorescent light). The blue/green fluorescence ratio, called the response ratio, provides a measure of the activation of hCCR5 (FIG. 3). 3 nM recombinant CCL3L1 was used as a positive control. A sample of ExpiFectamine 293 reagent treated cells (transfection control) was included as a negative control.

The results presented in FIG. 3 demonstrate that the protein encoded by TECH011-CV006 comprising a D27A-mutated hCCL3L1 targeting unit and the protein encoded by VB1020 which comprises a wild type hCCL3L1 targeting unit both bind to and activate the hCCR5 receptor. Thus, the introduction of the mutation into the targeting unit of the protein did not affect the binding of the targeting unit in said protein to the hCCR5 receptor or the activation thereof.

### C. Characterization of the Intact Proteins Encoded by VB1020 and TECH011-CV006 DNA Plasmids

Western blot (WB) analysis was performed on supernatant samples from Expi293F cells transfected with VB1020 and TECH011-CV006, respectively, to further characterize the proteins encoded by said DNA plasmids.

The samples were prepared by mixing 70 μL supernatant from transfected Expi293F cells with 25 μL 4× Laemmli sample buffer (Bio-Rad) with 5 μL DTT (Thermo Fisher Sci.) for reducing conditions. The samples were heated at 70 °C for 10 minutes before adding 25 μL per lane to 4%-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH-activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1α (AF270, R&D Systems) or mouse anti-E6 Papillomavirus Type 16 antibody (IG-E6-6F4, Euromedex). The membranes were washed, incubated with Dylight 800-conjugated anti-goat or anti-mouse secondary antibodies for 1 h at RT, and then washed and dried. Images were acquired by using a ChemiDoc™ MP Imaging System (setting Dylight 650 and 800, Auto Optimal). A sample of ExpiFectamine 293 reagent treated cells (transfection control) was included as a negative control.

The WB analysis confirmed the ELISA results of Example 1A, demonstrating that an intact protein was expressed from DNA plasmids VB1020 and TECH011-CV006, respectively (FIGS. 4 and 5). In addition, and in line with the ELISA results, a stronger WB signal was detected for the protein expressed from TECH011-CV006 than for the protein expressed from VB1020. Since the stronger WB signal was detected in both assays, using an anti-MIP-1α antibody and an anti-E6 HPV16 antibody, the stronger signal is not due to higher affinity of the anti-MIP-1α antibody to the wild type hCCL3L1 targeting unit compared to its affinity to the D27A-mutated hCCL3L1 targeting unit, but due to secretion of higher amounts of the protein comprising the D27A-mutated hCCL3L1 targeting unit compared to the protein comprising the wild type targeting unit.

Taken together, the ELISA and western blot data demonstrate that intact proteins can be expressed from DNA plasmids VB1020 and TECH011-CV006 and the proteins are secreted from transfected Expi293F cells. The data further show that secretion of the protein encoded by TECH011-CV006, comprising a hCCL3L1 targeting unit with a D27A mutation, is increased compared to that of the protein comprising a wild type hCCL3L1 targeting unit.

### Example 2

DNA plasmids VB2060, VB4231, TECH011-IV002 and TECH011-IV011 which were designed, produced and tested in Example 2 comprise nucleic acid sequences encoding the elements/units listed in Table 1 and further comprise nucleic acid sequences encoding the elements/units listed in Table 3 below:

TABLE 3

| Elements/ | VB2060 | VB4231 | TECH011-IV002 | TECH011-IV011 |
|---|---|---|---|---|
| Units | SEQ ID NO: 121 | SEQ ID NO: 122 | SEQ ID NO: 123 | SEQ ID NO: 124 |
| Targeting | hCCL3L1 wild type | hCCL3L1 wild type | hCCL3L1 with | D27AhCCL3L1 with |
| unit | SEQ ID NO: 2 | SEQ ID NO: 2 | mutation | D27A mutation |
| | | | SEQ ID NO: 5 | SEQ ID NO: 5 |
| Antigenic | SARS-CoV-2 receptor-binding domain | | | |
| unit | (RBD) SEQ ID NO: 125 | | | |
| Affinity | none | EPEA | none | EPEA |
| tag | | SEQ ID NO: 126 | | SEQ ID NO: 126 |

DNA plasmids VB2060, VB4231, TECH011-IV002 and TECH011-IV011 comprise nucleic acid sequences encoding for a protein comprising a hCCL3L1 targeting unit (wild type or comprising a D27A mutation), dimerization unit and an antigenic unit comprising the SARS-CoV-2 receptor-binding domain (RBD) antigen.

TECH011-IV002 and TECH011-IV011 are DNA plasmids comprising embodiments of constructs disclosed herein for use in the treatment of infectious diseases, i.e. constructs that comprise an antigenic unit comprising antigens derived from a pathogen (here: antigens derived from SARS-CoV-2). VB2060 and VB4231 serve as a control comprising the identical nucleic acid sequence as TECH011-IV002 and TECH011-IV011, respectively, but for the D27A mutation in the sequence of the hCCL3L1 targeting unit. VB4231 and TECH011-IV011 comprising identical sequences as VB2060 and TECH011-IV011 but for a sequence encoding a C-terminal EPEA-affinity-tag for protein purification.

### A. Assessment of Expression and Secretion of the Proteins Encoded by DNA Plasmids VB2060 and TECH011-IV002

Expi293F cells were transiently transfected with VB2060 and TECH011-IV002 as described in Example 1A.

The proteins present in the supernatants expressed from the DNA plasmids were characterized in a sandwich ELISA using mouse anti-human IgG CH3 domain antibody (capture antibody, 100 μL/well, 1 μg/mL, MCA878G, Bio-Rad) and goat anti-human MIP-1a antibody (biotinylated detection antibody, 100 μL/well, 0.2 μg/mL, BAF270, R&D systems). Supernatants collected from cells transfected with VB2060 were diluted 1:2000 and from cells transfected with TECH011-IV002 were diluted 1:4000, respectively. Protein concentration in the cell supernatant was interpolated from a standard curve of purified fusion protein as described in Example 1A. A sample of ExpiFectamine 293 reagent treated cells (transfection control) was included as a negative control.

The results presented in FIG. 6 demonstrate that the proteins encoded by VB2060 and TECH011-IV002 were well expressed and secreted from transfected Expi293F cells. The data further show that secretion of the protein comprising a D27A-mutated hCCL3L1 targeting unit, which was expressed from TECH011-IV002, is increased compared to secretion of the protein comprising the wild type hCCL1L3 targeting unit, which was expressed from VB2060.

### B. Assessment of Receptor Activation of Proteins Encoded by VB2060 and TECH011-IV002 DNA Plasmids

The hCCR5 reporter assay was performed with cell supernatant from Example 2A using a commercially available kit (Thermo Fisher Scientific, K1788) according to the manufacturer's protocol and as described in Example 1B.

The results presented in FIG. 7 demonstrate that the protein encoded by TECH011-IV002 comprising a D27A-mutated hCCL3L1 targeting unit and the protein encoded by VB2060 which comprises a wild type hCCL3L1 targeting unit both bind to and activate the hCCR5 receptor. Thus, the introduction of the mutation into the targeting unit of the protein did not affect the binding of the targeting unit in said protein to the hCCR5 receptor or the activation thereof.

C. Characterization of the Intact Proteins Expressed from VB2060 and TECH011-IV002 DNA Plasmids

WB analysis was performed on supernatant samples from Example 2A to further characterize the proteins encoded by VB2060 and TECH011-IV002.

The samples were prepared and run on 4%-20% Criterion TGX Stain-Free precast gels (Bio-Rad) as described in Example 1. Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1α (AF270, R&D Systems). The membranes were washed, incubated with fluorochrome-conjugated anti-goat secondary antibodies for 1 h at RT, and then washed and dried. Images were acquired by using a ChemiDoc™ MP Imaging System (setting Dylight 650 and 800, Auto Optimal). Membranes were re-activated in ethanol and re-blocked in EveryBlot buffer (Bio-Rad). Membranes were probed with rabbit anti-SARS-CoV-2 (2019-nCoV) Spike RBD (40592-T62, Sino Biological). The membranes were washed, incubated with Dylight 650-conjugated anti-rabbit secondary antibodies for 1 h at RT, and then washed and dried. Images were acquired by using a ChemiDoc™ MP Imaging System (setting Dylight 650, Auto Optimal). A sample of ExpiFectamine 293 reagent treated cells (transfection control) was included as a negative control.

The WB analysis confirmed the ELISA results of Example 2A, demonstrating that intact proteins were expressed from DNA plasmids VB2060 and TECH011-IV002, respectively (FIGS. 8 and 9). In addition, and in line with the ELISA results, a stronger WB signal was detected for the protein expressed from TECH011-IV002 than for the protein expressed from VB2060. Since the stronger WB signal was detected in both assays, using an anti-MIP-1α antibody and an anti-Spike RBD antibody, the stronger signal is not due to higher affinity of the anti-MIP-1α antibody to the D27A-mutated hCCL3L1 targeting unit, but due to secretion of higher amounts of protein comprising the D27A-mutated hCCL3L1 targeting unit, compared to that comprising the wild type hCCL3L1 targeting unit.

Taken together, the ELISA and western blot data demonstrate that intact proteins can be expressed from DNA plasmids TECH011-IV002 and VB2060 and that the proteins are secreted from transfected Expi293F cells. The data further show that secretion of the protein encoded by TECH011-IV002 comprising a hCCL3L1 targeting unit with a D27A mutation is increased, compared to that of the protein comprising a hCCL3L1 targeting unit without the D27A mutation.

D. Purification of Proteins Expressed from DNA Plasmids VB4231 and TECH011-IV011 and Analysis of the Purified Proteins

Expi293F cells were transiently transfected with VB4231 and TECH011-IV011. Briefly, Expi293F cells (2.71×10⁶ cells/mL, 60 mL per flask) were seeded in two 250 mL flasks for each plasmid. The cells were transfected with 0.66 μg/mL plasmid DNA using ExpiFectamine 293 Reagent (Thermo Fisher Scientific), and the flasks were incubated on an orbital shaker (19 mm diameter, 125 rpm) in a humidified CO₂ cell incubator (8% CO₂, 37 °C) for 70 h before the supernatant was harvested.

The content of supernatant was purified by affinity tag purification based on the presence of the EPEA tag in the protein. Briefly, equilibrated CaptureSelect™ C-tagXL Affinity Matrix (Thermo Fisher Sci.) was incubated with Expi293F cell supernatant for 1 h and washed with Tris-buffered saline (TBS) and 25 mM Tris-HCl pH 7.2, 1 M NaCl, 0.05% Tween. Proteins were eluted with 2 mM SEPEA peptide in TBS. Four eluate fractions were combined before buffer exchange and spin concentration using 10-kDa MWCO Pierce™ Protein concentrators (Thermo Fisher Scientific). Samples were taken from cell supernatant, flow-through, wash fractions and elution fractions for SDS-PAGE analysis.

The samples were prepared by mixing 32.5 μL cell supernatant or purification fractions with 12.5 μL 4× Laemmli sample buffer (Bio-Rad) with 5 μL DTT (Cayman Chemical) for reducing conditions. The samples were heated at 80 °C for 10 minutes before adding 25 μL per lane to 4%-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). The SDS-gels were incubated with InstantBlue™ Coomassie Stain (Abcam) and de-stained in water. Images were acquired by using a ChemiDoc™ MP Imaging System. Protein concentration was determined by measuring absorbance at 280 nm (A280) using a NanoDrop Fluorospectrometer.

The SDS-PAGE analysis of the above-mentioned fractions presented in FIGS. 10A and B, demonstrate that intact proteins were expressed from DNA plasmids VB4231 (EPEA-tagged VB2060) and TECH011-IV011 (EPEA-tagged TECH011-IV002), respectively. In addition, and in line with the ELISA and WB results, stronger signal protein bands were observed for protein expressed from TECH011-IV011 than for that expressed from VB4231 (FIG. 10). Protein concentration measurement (A280) further confirmed that TECH011-IV011 (16.7 mg per liter of culture) resulted in a 5-fold higher purification yield compared to VB4231 (3.3 mg per liter of culture).

E. Determination of the Oligomerization State of the Purified Proteins Expressed from VB4231 and TECH011-IV011 DNA Plasmids

For SDS-PAGE analysis of the purified proteins obtained in Example 2D, proteins were diluted corresponding to 0.5 μg and 2 μg protein (based on A280 protein concentration measurement) and mixed with 4× Laemmli sample buffer (Bio-Rad) and DTT (Thermo Fisher Scientific) or water for reducing and non-reducing conditions, respectively. The samples were heated at 80 °C for 10 minutes before loading 20 μL per lane to a 4%-20% Criterion TGX Stain-Free™ Precast Gel (Bio-Rad). SDS-PAGE was performed in 1× Tris/Glycine/SDS running buffer (Bio-Rad) with Precision Plus Protein All Blue Prestained and Unstained protein standards mix (Bio-Rad). The SDS-gel was incubated with InstantBlue™ Coomassie Stain (Abcam) and destained in water. Images were acquired by using a ChemiDoc™ MP Imaging System.

The SDS-PAGE analysis of the purified proteins presented in FIGS. 11A and B demonstrates that the protein expressed from TECH011-IV011 shows fewer high-molecular bands under non-reducing conditions compared to the protein expressed from VB4231, indicating a reduction in oligomerization, which is due to the presence of the D27A mutation in its hCCL3L1 targeting unit.

To investigate protein oligomerization state in solution, the purified proteins (100 μL, 2 mg/mL protein concentration) were subjected to size-exclusion chromatography (SEC) using TBS and an SD200 Increase 10/300 GL column (Cytiva) mounted on an AKTA Avant (Cytiva).

The size exclusion chromatogram presented in FIGS. 12A and B shows that protein expressed from TECH011-IV011 shows a peak at a smaller molecular size than protein expressed from VB4231, confirming a reduction in oligomerization in solution for the protein comprising the D27A mutated hCCL3L1 targeting unit compared to the protein comprising the wild type hCCL3L1 targeting unit. The data further show that protein expressed from TECH011-IV011 (FIG. 12B) was more homogeneous, compared to that expressed from VB4231 (FIG. 12A).

### Example 3

DNA plasmids VB4097 and TECH011-CV007 which were designed, produced and tested in Example 3 comprise nucleic acid sequences encoding the elements/units listed in Table1 and further comprise nucleic acid sequences encoding the units listed in Table 4 below:

TABLE 4

| Elements/ | VB4097 | TECH011-CV007 |
|---|---|---|
| Units | SEQ ID NO: 127 | SEQ ID NO: 128 |
| Targeting | hCCL3L1 wild type | hCCL3L1 with D27A mutation |
| unit | SEQ ID NO: 2 | SEQ ID NO: 5 |

| | |
|---|---|
| Antigenic | 10 CT26 epitopes separated by glycine- |
| unit | serine linkers (see Table 5) |

Previously described exome sequencing and RNA sequencing of the mouse colon cancer cell line CT26 revealed hundreds to thousands of tumor-specific non-synonymous mutations. In silico methods were used to identify potential immunogenic sequences, i.e. epitopes comprising a mutation, and 10 of them (Table 5) were chosen for inclusion into the antigenic unit of the protein encoded by the above-mentioned DNA plasmids. The epitopes in said antigenic unit are separated by glycine-serine linkers (GGGGSGGGGS, SEQ ID NO: 28), i.e. all epitopes but the terminal epitope are arranged in subunits, each subunit consisting of one epitope and one GGGGSGGGGS linker.

DNA plasmid TECH011-CV007 is a model of a DNA plasmid comprising a construct as disclosed herein for use in the individualized cancer treatment of a specific patient, i.e. one that comprises an antigenic unit comprising several patient-specific epitopes, e.g. several neoepitopes and/or several patient-present shared cancer epitopes, with the patient-present shared cancer antigens being mutated patient-present shared cancer antigens. It is however also a model of a DNA plasmid comprising construct as disclosed herein for use as off-the-shelf treatment of cancer, i.e. one that comprises an antigenic unit comprising several shared cancer epitopes, with the shared cancer antigens being mutated shared cancer antigens.

TABLE 5

| Epitope | Gene | Sequence | SEQ ID NO: |
|---|---|---|---|
| C-pepM01 | E2f8 | VILPQAPSGPSYATYLQPAQAQMLTPP | 129 |
| C-pepM06 | UbqIn1 | DTLSAMSNPRAMQVLLQIQQGLQTLAT | 130 |
| C-pepM08 | Dhx35 | EVIQTSKYYMRDVIAIESAWLLELAPH | 131 |
| C-pepM29 | Anapc1 | GSLFGSSRVQYVVNPAVKIVFLNIDPS | 132 |
| C-pepM31 | Ptpn13 | AEYGDYQPEVHGVPYFRLEHYLPARVM | 133 |
| C-pepM43 | Mtch1 | KSWIHCWKYLSVQSQLFRGSSLLFRRV | 134 |
| C-pepM89 | Mmachc | TLAFLVLSTPAMFNRALKPFLKSCHFQ | 135 |
| C-pepM149 | 3110057O12Rik | FVSPMAHYVPGIMAIESVVARFQFIVP | 136 |
| C-pepM171 | Gdf11 | LWVYLRPVPRPATIYLQILRLKPLTGE | 137 |
| C-pepM173 | Top3a | KIYEFDYHLYGQNITMIMTSVSGHLLA | 138 |

### A. Assessment of Expression and Secretion of the Proteins Encoded by VB4097 and TECH011-CV007 DNA Plasmids

Expi293F cells were transiently transfected with VB4097 and TECH011-CV007 as described in Example 1A. The proteins present in the supernatants which were expressed from the DNA plasmids were characterized in a sandwich ELISA using mouse anti-human IgG CH3 domain antibody (capture antibody, 100 μL/well, 1 μg/mL, MCA878G, Bio-Rad) and goat anti-human MIP-1α antibody (biotinylated detection antibody, 100 μL/well, 0.2 μg/mL, BAF270, R&D systems) (FIG. 13). Protein concentration in the cell supernatant was interpolated from a standard curve of purified fusion protein as described in Example 1A. A sample of ExpiFectamine 293 reagent treated cells (transfection control) was included as a negative control.

The results presented in FIG. 13 demonstrate that the proteins encoded in VB4097 and TECH011-CV007 were well expressed and secreted from transfected Expi293F cells. The data further show that secretion of the protein comprising a D27A-mutated hCCL1L3 targeting unit expressed from TECH011-CV007 was markedly increased compared to that of the protein comprising a wild type hCCL3L1 targeting unit expressed from VB4097.

### B. Assessment of Receptor Activation of Proteins Encoded by DNA Plasmids

The hCCR5 reporter assay was performed with cell supernatant from Example 3A using a commercially available kit (Thermo Fisher Scientific, K1788) according to the manufacturer's protocol and as described in Example 1B.

The results presented in FIG. 14 demonstrate that that the protein encoded by TECH011-CV007 comprising a D27A mutated hCCL3L1 targeting unit and the protein encoded by VB4097 which comprises a wild type hCCL3L1 targeting unit both bind to and activate the hCCR5 receptor. Thus, the introduction of the mutation into the targeting unit of the protein did not affect the binding of the targeting unit in said protein to the hCCR5 receptor or the activation thereof.

C. Characterization of the Intact Proteins Expressed from VB4097 and TECH011-CV007 DNA Plasmids

WB analysis was performed on supernatant samples from Example 3A to further characterize the proteins encoded by VB4097 and TECH011-CV007.

The samples were prepared and run on 4%-20% Criterion TGX Stain-Free precast gels (Bio-Rad) as described in Example 1. Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with goat anti-human MIP-1α (AF270, R&D Systems). The membranes were washed, incubated with fluorochrome-conjugated anti-goat secondary antibodies for 1 h at RT, and then washed and dried. Images were acquired by using a ChemiDoc™ MP Imaging System (setting Dylight 650 and 800, Auto Optimal). A sample of ExpiFectamine 293 reagent treated cells (transfection control) was included as a negative control.

The WB analysis confirmed the ELISA results of Example 3A, demonstrating that intact proteins were expressed from DNA plasmids VB4097 and TECH011-CV007 (FIG. 15). In addition, and in line with the ELISA results, a stronger WB signal was detected for the protein expressed from TECH011-CV007 than for that expressed from VB4097.

Taken together, the ELISA and western blot data demonstrate that intact proteins can be expressed from DNA plasmids VB4097 and TECH011-CV007 and the proteins are secreted from transfected Expi293F cells. The data further show that secretion of the protein encoded by TECH011-CV007 comprising a hCCL3L1 targeting unit with a D27A mutation is increased, compared to that of the protein encoded by VB4097, comprising a hCCL3L1 targeting unit without the D27A mutation.

### Example 4

The following DNA plasmids were designed and produced:

All DNA plasmids designed, produced and tested in Example 4 comprise nucleic acid sequences encoding the elements/units listed in Table 1 and further comprise nucleic acid sequences encoding the elements/units listed in Table 6 below.

TABLE 6

| Element/ | VB1020 | TECH011-CV006 | TECH011-CV036 |
|---|---|---|---|
| Units | SEQ ID NO: 118 | SEQ ID NO: 119 | SEQ ID NO: 142 |
| Targeting | hCCL3L1 wild | hCCL3L1 with | hCCL3L1 with D27A |
| unit | type | D27A mutation | and E67A mutations |
| | SEQ ID NO: 2 | SEQ ID NO: 5 | SEQ ID NO: 8 |

| | |
|---|---|
| Antigenic | HPV16 E7 antigen and HPV16 E6 antigen |
| unit | separated by a glycine-serine linker |
| | SEQ ID NO: 120 |

DNA plasmids VB1020, TECH011-CV006, and TECH011-CV036 comprise nucleic acid sequences encoding a protein comprising a hCCL3L1 targeting unit (either wild type, comprising a D27A mutation or comprising a D27A and E67A mutation), a dimerization unit, and an antigenic unit comprising human papilloma virus 16 (HPV16) antigens E7 and E6.

TECH011-CV006 and TECH011-CV036 are DNA plasmids comprising an embodiment of a nucleotide sequence as disclosed herein, encoding a non-individualized construct for use in the treatment of cancer, i.e. one that comprises an antigenic unit comprising several shared cancer antigens, with the shared cancer antigens being viral shared cancer antigens (here: antigens from HPV16 which causes certain types of cancer). They are, however, also DNA plasmids comprising an embodiment of a nucleotide sequence as disclosed herein, encoding a construct for use in the treatment of infectious diseases, i.e. one that comprises an antigenic unit comprising antigens derived from a pathogen (here: antigens derived from HPV16). VB1020 serves as a control comprising the identical nucleic acid sequence as TECH011-CV006 and TECH011-CV036, but for the D27A mutation or D27A and E67A mutation in the sequence of the hCCL3L1 targeting unit.

### A. Assessment of Expression and Secretion of Proteins Encoded by VB1020, TECH011-CV006 and TECH011-CV036 DNA Plasmids

Expi293F cells were seeded and transfected as described in Example 1A. The plates were incubated for 1 or 3 days before the supernatant was harvested.

The proteins present in the supernatants expressed from the DNA plasmids were characterized in a sandwich ELISA as described in Example 1A. Supernatants collected from cells transfected with VB1020 were diluted 1:50/1:500, and from cells transfected with TECH011-CV006 and TECH011-CV036 were diluted 1:500/1:2000, respectively. Protein concentration in the cell supernatant was interpolated from a standard curve of purified protein as described in Example 1A. A sample of ExpiFectamine 293 reagent treated cells was included as a negative control.

FIG. 16 shows the amount of protein secreted from cells transfected with the DNA plasmids on day 3 after transfection. The proteins encoded by VB1020, TECH011-CV006, and TECH011-CV036 were well expressed and secreted from transfected Expi293F cells. The data further show that secretion of the protein comprising a D27A mutated hCCL3L1 targeting unit which was expressed from TECH011-CV006-transfected cells and secretion of the protein comprising a D27A and a E67A mutated hCCL3L1 targeting unit which was expressed from TECH011-CV036 transfected cells is markedly increased, compared to secretion of the protein comprising the wild type hCCL1L3 targeting unit.

FIG. 17 shows a comparison of amount of protein secreted from cells transfected with the DNA plasmids on days 1 and 3 after transfection, respectively. The results demonstrate that secretion of the protein comprising a D27A mutated hCCL3L1 targeting unit which was expressed from TECH011-CV006-transfected cells and secretion of the protein comprising a D27A and a E67A mutated hCCL3L1 targeting unit which was expressed from TECH011-CV036 transfected cells on day 1 after transfection was markedly increased when compared to secretion of the protein comprising the wild type hCCL1L3 targeting unit on day 3 after transfection. In conclusion, proteins comprising the above-described mutated hCCL3L1 targeting units were not only secreted at higher amounts from transfected Expi293F cells, but also faster, compared to the protein comprising the wild type hCCL3L1 targeting unit.

### B. Characterization of the Intact Proteins Encoded by VB1020, TECH011-CV006 DNA Plasmids and TECH011-CV036

WB analysis was performed to further characterize the proteins encoded by said DNA plasmids.

Supernatant samples from Expi293F cells transfected with TECH011-CV006 and TECH011-CV036 were diluted 1:10 in Expi293 expression medium prior to SDS-PAGE sample preparation. The samples were prepared by mixing 45.5 μL supernatant from transfected Expi293F cells with 17.5 μL 4× Laemmli sample buffer (Bio-Rad) with 7 μL DTT (Thermo Fisher Sci.) for reducing conditions. The samples were heated at 80 °C for 10 minutes before adding 35 μL per lane to 4-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH-activated low fluorescence (LF) 0.45 μm PVDF membranes (Bio-Rad) by using the Trans-Blot Turbo semi-dry transfer system (Bio-Rad). PVDF membranes were blocked in EveryBlot buffer (Bio-Rad) for 5 min and probed with goat anti-human MIP-1α (AF270, R&D Systems). The membranes were washed, incubated with Dylight 550-conjugated anti-goat secondary antibodies (Thermo Fisher) for 1 h at RT, and then washed and dried. Images were acquired by using a ChemiDoc™ MP Imaging System (setting Dylight 650 and 550, Auto Optimal). A sample of ExpiFectamine 293 reagent treated cells (transfection control) was included as a negative control.

The WB analysis confirmed the ELISA results of Example 4A, demonstrating that intact proteins were expressed from the DNA plasmids VB1020, TECH011-CV006 and TECH011-CV036 (FIG. 18). In addition, and in line with the ELISA results, the results of a quantification of the band intensities of the reduced WB show that a much stronger WB signal was detected for the protein expressed from TECH011-CV006 and TECH011-CV036 than for the protein expressed from VB1020 (FIG. 19).

Taken together, the ELISA and western blot data demonstrate that intact proteins were expressed from DNA plasmids VB1020, TECH011-CV006 and TECH011-CV036 and the proteins were secreted from Expi293F cells transfected with the DNA plasmids. The data further show that secretion of the proteins comprising a hCCL3L1 targeting unit with a D27A mutation or a D27A and a E67A was accelerated and increased compared to that of the protein comprising a wild type hCCL3L1 targeting unit.

### C. Assessment of Immunogenicity of VB1020 and TECH011-CV006

Immunogenicity of TECH011-CV006 was determined and compared to immunogenicity of VB1020. DNA plasmid VB1026 comprises a nucleic acid sequence encoding a protein comprising the signal peptide of Table 1, the wild type hCCL3L1 targeting unit of SEQ ID NO: 2 and the dimerization unit of Table 1, but no unit linker and no antigenic unit. It was used as negative control.

Female, 6-week-old C57BL/6 mice were obtained from Janvier Labs (France). All animals were housed in the animal facility at the Oslo University Hospital (Oslo, Norway). All animal protocols were approved by the Norwegian Food Safety Authority (Oslo, Norway). 5 mice/group were used for the testing of TECH011-CV006 and VB1020, whereas 3 mice/group were used for the negative control (VB1026).

Doses of 0.5, 1, 5, or 25 μg of the DNA plasmids VB1020 and TECH011-CV006 or 25 μg DNA plasmid VB1026 were administered to C57BL/6 mice intramuscularly (each tibialis anterior, 2×25 μL) followed by electroporation with AgilePulse in vivo electroporation system (BTX, USA) on day 0 and on day 21. The spleens were collected 7 days after the second administration and was mashed in a cell strainer to obtain a single cell suspension. Total splenocytes were then tested for production of INF-γ and TNF-α in a FluoroSpot assay according to the manufactures protocol (Mabtech).

A single peptide of E7 and a peptide pool of peptides of E6 as listed in Table 7 were used to re-stimulate the splenocytes harvested from mice administered with VB1020 and TECH011-CV006.

TABLE 7

| Re-stimulation | Gene | Sequence | SEQ ID NO |
|---|---|---|---|
| Single peptide | HPV16 E7 | RAHYNIVTF | 143 |
| | (49-57) | | |
| Peptide pool | HPV16 E6 | MFQDPQERPRKLPQL | 144 |
| | | RPRKLPQLCTELQTT | 145 |
| | | LCTELQTTIHDIILE | 146 |
| | | TIHDIILECVYCKQQ | 147 |
| | | ECVYCKQQLLRREVY | 148 |
| | | QLLRREVYDFARRDL | 149 |
| | | YDFARRDLCIVYRDG | 150 |
| | | LCIVYRDGNPYAVRD | 151 |
| | | GNPYAVRDKCLKFYS | 152 |
| | | DKCLKFYSKISEYRH | 153 |

No IFN-γ (FIG. 20), TNF-α (FIG. 21) or IFN-γ and TNF-α (FIG. 22) production was detected in response to VB1026 administration. While VB1020 elicited strong T cell responses against the E7 and E6 peptides, TECH011-CV006 elicited even stronger T cell responses compared to VB1020 for all doses (FIGS. 20, 21 and 22), particularly for low doses, i.e. 0.5 and 1 μg (FIGS. 20, 21, 22 and (FIG. 23, which only compares the 1 μg dose). The mean T cell response elicited by 0.5 μg TECH011-CV006 was at the same level as the mean T cell response elicited by 1 μg VB1020, and similarly, 1 μg TECH011-CV006 elicited T cell responses at a similar level as 5 μg VB1020 (FIGS. 20, 21, 22). These observations suggest that a lower dose of the DNA plasmid TECH011—CV006 comprising a D27A mutated hCCL3L1 targeting unit can elicit an equally strong immune response as a higher dose of the DNA plasmid comprising the wild type hCCL3L1 targeting unit.

### Example 5

DNA plasmids VB2060, TECH011-IV002 and TECH011-IV015 which were designed, produced and tested in this Example 5 comprise nucleic acid sequences encoding the elements/units listed in Table 1 and further comprise nucleic acid sequences encoding the elements/units listed in Table 8 below:

TABLE 8

| Elements/ | VB2060 | TECH011-IV002 | TECH011-IV015 |
|---|---|---|---|
| Units | SEQ ID NO: 121 | SEQ ID NO: 123 | SEQ ID NO: 154 |
| Targeting | hCCL3L1 wild | hCCL3L1 with | hCCL3L1 with D27A |
| unit | type | D27A mutation | and E67A mutations |
| | SEQ ID NO: 2 | SEQ ID NO: 5 | SEQ ID NO: 8 |

| | |
|---|---|
| Antigenic | SARS-CoV-2 receptor-binding domain |
| unit | (RBD) SEQ ID NO: 125 |

DNA plasmids VB2060, TECH011-IV002 and TECH011-IV015 comprise nucleic acid sequences encoding for a protein comprising a hCCL3L1 targeting unit (either wild type or comprising a D27A or D27A and E67A mutation), a dimerization unit and an antigenic unit comprising the SARS-CoV-2 receptor-binding domain (RBD) antigen.

TECH011-IV002 and TECH011-IV015 are DNA plasmids comprising embodiments of nucleotide sequences as disclosed herein, encoding constructs for use in the treatment of infectious diseases, i.e. constructs that comprise an antigenic unit comprising antigens derived from a pathogen (here: antigens derived from SARS-CoV-2). VB2060 serves as a control comprising the identical nucleic acid sequences as TECH011-IV002 and TECH011-IV015, but for the D27A mutation or D27A and E67A mutations in the sequence of the hCCL3L1 targeting unit, respectively.

### A. Assessment of Expression and Secretion of the Proteins Encoded by DNA Plasmids VB2060, TECH011-IV002 and TECH011-IV015

Expi293F cells were transiently transfected with VB2060, TECH011-IV002 and TECH011-IV015 as described in Example 1A. The plates were incubated for 1 or 3 days before the supernatant was harvested.

The proteins present in the supernatants expressed from the DNA plasmids were characterized in a sandwich ELISA using mouse anti-human IgG CH3 domain antibody (capture antibody, 100 μL/well, 1 μg/mL, MCA878G, Bio-Rad) and goat anti-human MIP-1a antibody (biotinylated detection antibody, 100 μL/well, 0.2 μg/mL, BAF270, R&D systems). Supernatants collected from cells transfected with VB2060 were diluted 1:500/1:1500, samples from cells transfected with TECH011-IV002 were diluted 1:1500/1:20000 and 1:20000 for the day 1 and day 3 samples, respectively, and from cells transfected with TECH011-IV015 were diluted 1:1500/1:20000 and 1:20000 for the day 1 and day 3 samples, respectively. Protein concentration in the cell supernatant was interpolated from a standard curve of purified protein as described in Example 1A. A sample of ExpiFectamine 293 reagent treated cells was included as a negative control.

FIG. 24 shows the amount of protein secreted from cells transfected with the DNA plasmids on day 3 after transfection. The proteins encoded by VB2060, TECH011-IV002, and TECH011-IV015 were well expressed and secreted from transfected Expi293F cells. The data further show that secretion of the protein comprising a D27A mutated hCCL3L1 targeting unit which was expressed from TECH011-IV002-transfected cells and secretion of the protein comprising a D27A and a E67A mutated hCCL3L1 targeting unit which was expressed from TECH011-IV015-transfected cells was markedly increased, compared to secretion of the protein comprising the wild type hCCL1 L3 targeting unit.

FIG. 25 shows a comparison of amount of protein secreted from cells transfected with the DNA plasmids on days 1 and 3 after transfection, respectively. The results demonstrate that secretion of the protein comprising a D27A mutated hCCL3L1 targeting unit which was expressed from TECH011-IV002-transfected cells and secretion of the protein comprising a D27A and a E67A mutated hCCL3L1 targeting unit which was expressed from TECH011-IV015-transfected cells on day 1 after transfection was markedly increased when compared to secretion of the protein comprising the wild type hCCL1L3 targeting unit on day 3 after transfection. In conclusion, proteins comprising the above-described mutated hCCL3L1 targeting units were not only secreted at higher amounts from transfected Expi293F cells, but also faster, compared to the protein comprising the wild type hCCL3L1 targeting unit.

B. Characterization of the Intact Proteins Expressed from VB2060, TECH011-IV002 and TECH011-IV015 DNA Plasmids

WB analysis was performed to further characterize the proteins encoded by VB2060, TECH011-IV002 and TECH011-IV015. The analysis was carried out as described in Example 4B, but for non-reducing conditions, water was used instead of DTT.

The WB analysis confirmed the ELISA results of Example 5A, demonstrating that intact proteins were expressed from the DNA plasmids. The non-reduced WB analysis further confirms that all 3 proteins form dimers. (FIG. 26). In addition, and in line with the ELISA results, the results of a quantification of the band intensities of the reduced WB show that a much stronger WB signal was detected for the protein expressed from TECH011-IV002 and TECH011-IV015 than for the protein expressed from VB2060 (FIG. 27).

Taken together, the ELISA and western blot results demonstrate that intact proteins were expressed from DNA plasmids VB2060, TECH011-IV002, and TECH011-IV015 and that the proteins were secreted from transfected Expi293F cells. The results further show that secretion of the proteins comprising a hCCL3L1 targeting unit with a D27A mutation or with a D27A and a E67A mutation was accelerated and increased compared to that of the protein comprising a wild type hCCL3L1 targeting unit.

### C. Assessment of Humoral Immune Response Induced in Mice Against SARS-CoV-2 RBD

Humoral immune response induced in mice against SARS-CoV2 RBD upon administration of TECH011-IV002 was determined and compared to that induced upon administration of VB2060. VB1026 (described in Example 4C) was used as a negative control.

Female, 6-week-old BALB/c mice were obtained from Janvier Labs (France). All animals were housed in the animal facility at the Oslo University Hospital (Oslo, Norway). All animal protocols were approved by the Norwegian Food Safety Authority (Oslo, Norway). 6 mice/group were used for the testing of TECH011-IV002 and VB2060, whereas 5 mice/group were used for the negative control.

A dose of 1 μg DNA plasmid was administered on day 0 and on day 21 as described in Example 4C. Sera were collected 13 days after the second administration and tested for presence of anti-RBD IgG antibodies binding the RBD protein (Wuhan variant).

Briefly, blood was collected from the saphenous vein of the mice. Coagulated blood was centrifuged twice (1000 g, 15 min) and the serum was collected and transferred to a clean tube. The humoral immune response was evaluated in an ELISA assay detecting total IgG in the sera binding to RBD (aa319-542) from SARS-CoV2 (Wuhan variant). ELISA plates (MaxiSorp Nunc-Immuno plates) were coated with 1 μg/mL recombinant RBD-His protein antigen in PBS overnight at 4 °C. Plates were blocked with 4% BSA in PBS for 1 h at RT. Plates were then incubated with serial dilutions of mouse sera (diluted in 0.1% BSA in PBS) and incubated for 2 h at 37 °C. Plates were washed 3× and incubated with 1:50 000 dilution of anti-mouse total IgG-HRP antibody (Southern Biotech) in 0.1% BSA in PBS and incubated for 1 h at 37 °C. After final washing, plates were developed using TMB substrate (Merck, cat. CL07-1000). Plates were read at 450 nm wavelength within 30 min using a SPARK® Multimode Microplate Reader (Tecan). Binding antibody endpoint titers were calculated as the reciprocal of the highest dilution resulting in a signal above the cutoff. RBD (Sino Biological 40592-V08H) was tested as the binding antigen.

The results shown in FIG. 28 demonstrate that DNA plasmid TECH011-IV002, encoding a protein comprising a hCCL3L1 targeting unit with a D27A mutation, induced stronger total IgG responses against RBD than DNA plasmid VB2060 encoding a protein comprising a wild type hCCL3L1 targeting unit in mice administered with the plasmids.

### Example 6

DNA plasmids MC38-I2-WT, MC38-I2-D27A, MC38-I2-E67A, and MC38-I2-D27A-E67A which were designed, produced and tested in this Example 6 comprise nucleic acid sequences encoding the elements/units listed in Table 1 and further comprise nucleic acid sequences encoding the elements/units listed in Table 9 below:

TABLE 9

| Elements/ | MC38-I2-WT | MC38-I2-D27A | MC38-I2-E67A | MC38-I2-D27A-E67A |
|---|---|---|---|---|
| Units | SEQ ID NO: 155 | SEQ ID NO: 156 | SEQ ID NO: 157 | SEQ ID NO: 158 |
| Targeting | hCCL3L1 wild | hCCL3L1 with | hCCL3L1 with | hCCL3L1 with D27A |
| unit | type | D27A mutation | E67A mutation | and E67A mutations |
| | SEQ ID NO: 2 | SEQ ID NO: 5 | SEQ ID NO: 6 | SEQ ID NO: 8 |

| | |
|---|---|
| Antigenic | 20 MC38 neoantigens separated by glycine-serine linkers (see Table 10) |
| unit | |

The DNA plasmids MC38-I2-WT, MC38-I2-D27A, MC38-I2-E67A, and MC38-I2-D27A-E67A all comprise a nucleic acid sequence that encodes an antigenic unit comprising 20 neoantigens from the MC38 mouse tumor cell line. The neoantigens are separated by glycine-serine linkers (GGGGSGGGGS, SEQ ID NO: 28), i.e. all neoantigens but the terminal neoantigen are arranged in 20 subunits, each subunit consisting of one neoantigen and one GGGGSGGGGS linker.

MC38-I2-D27A, MC38-I2-E67A and MC38-I2-D27A-E67A are DNA plasmids comprising embodiments of nucleotide sequences as disclosed herein for use in the treatment of cancer for a specific patient only (individualized anticancer constructs), i.e. encoding a polypeptide that comprises an antigenic unit comprising several patient-specific epitopes, i.e. several neoepitopes. The plasmids are, however, also models of a DNA plasmid comprising nucleotide sequences as disclosed herein for use as non-individualized anticancer constructs, i.e. encoding polypeptides that comprise an antigenic unit comprising several shared cancer epitopes, with the shared cancer antigens being shared cancer antigens comprising a mutation.

MC38-I2-WT serves as a control comprising the identical nucleic acid sequence as MC38-I2-D27A, MC38-I2-E67A and MC38-I2-D27A-E67A, but for the D27A and/or E67A mutations in the sequence of the hCCL3L1 targeting unit.

TABLE 10

| | | | SEQ ID |
|---|---|---|---|
| Epitope | Gene | Sequence | NO: |
| M002 | irgq | KARDETAALLNSAVLGAAPLFVPPADC | 159 |
| M007 | Atg9a | LSNRILWIGIANFQLCPLILIWQILYA | 160 |
| M010 | Reps1 | NGRVLELFRAAQLANDVVLQIMELCGA | 161 |
| M016 | Adpgk | GIPVHLELASMTNMELMSSIVHQQVFP | 162 |
| M020 | N4bp212 | NNRVAVATINFRRLVCPQEDKTSTDVL | 163 |
| M024 | Ccdc96 | AEPESPEKEQAYLGYLAMLEELKKQEA | 164 |
| M039 | Actr1b | ESEGLHEVLAFAILKSDMDLRRTLFSN | 165 |
| M046 | C130039O16Rik | TLPLQPFQLAFGHLVNRQVFRQGPQPS | 166 |
| M047 | Car7 | YMVRFKDTKAQFSFFNPKCLLPTSRHY | 167 |
| M086 | Rpl18 | RILKAGGKILTFDRLALESPKGRGTVL | 168 |
| M091 | Zbtb40 | VCKACDKSFHFYCPLKVHMKRCRVAKS | 169 |
| M100 | Fam46b | RGGGLLKYCHLLVLGFRPRPSTDVRAL | 170 |
| M111 | Aatf | SKLLSFMAPIDHTTMSDDARTELFRSL | 171 |
| M143 | Spire1 | GEKRSISAIRSYQYVMKICAAHLPTES | 172 |
| M152 | Dpagt | SLVISASIIVFNLLELEGDYRDDHIFS | 173 |
| M164 | Dtx31 | HVLPKCKHEFCTSSISKAMLIKPVCPV | 174 |
| M170 | cpne1 | VDFTGSNGDPSSPYSLHYLSPTGVNEY | 175 |
| M172 | Med12 | SMDIDPSSSVLFEYMEKPDFSLFSPTM | 176 |
| M186 | Mttp | SSSAYTGYVERSPLAASTYSLDILYSG | 177 |
| M222 | Hnrnpl | ITTDVLYTICNPCVPVQRIVIFRKNGV | 178 |

### A. Assessment of Expression and Secretion of the Proteins Encoded by DNA Plasmids MC38-I2-WT. MC38-I2-D27A. MC38-I2-E67A and MC38-I2-D27A-E67A

Expi293F cells were transiently transfected with MC38-I2-WT, MC38-I2-D27A, MC38-I2-E67A and MC38-I2-D27A-E67A as described in Example 1A. Expi293F cells were seeded and transfected as described in Example 1A. The plates were incubated for 1 or 3 days before the supernatant was harvested.

The proteins present in the supernatants expressed from the DNA plasmids were characterized in a sandwich ELISA using mouse anti-human IgG CH3 domain antibody (capture antibody, 100 μL/well, 1 μg/mL, MCA878G, Bio-Rad) and goat anti-human MIP-1α antibody (biotinylated detection antibody, 100 μL/well, 0.2 μg/mL, BAF270, R&D systems). Supernatants collected from cells transfected with MC38-I2-WT were diluted 1:2 or 1:2/1:10/1:50 for day 1 and day 3 samples, respectively, supernatants from cells transfected with MC38-I2-D27A were diluted 1:2/1:10 or 1:2/1:10/1:50 for day 1 and day 3 samples, respectively, supernatants from cells transfected with MC38-I2-E67A were diluted 1:2/1:10 or 1:2/1:10/1:50 for day 1 and day 3 samples, respectively, and supernatants from cells transfected with MC38-I2-D27A-E67A were diluted 1:2/1:10 or 1:2/1:10/1:50 for day 1 and day 3 samples, respectively. Protein concentration in the cell supernatant was interpolated from a standard curve of purified protein as described in Example 1A. A sample of ExpiFectamine 293 reagent treated cells was included as a negative control.

FIG. 29 shows the amount of protein secreted from cells transfected with the DNA plasmids on day 3 after transfection. The proteins encoded by MC38-I2-WT, MC38-I2-D27A, MC38-I2-E67A and MC38-I2-D27A-E67A were well expressed and secreted from transfected Expi293F cells. The data further show that secretion of the proteins comprising a hCCL3L1 targeting unit with a D27A and/or E67A mutation which were expressed from cells transfected cells with MC38-I2-D27A, MC38-I2-E67A or MC38-I2-D27A-E67A was markedly increased, compared to secretion of the protein comprising the wild type hCCL1 L3 targeting unit.

FIG. 30 shows a comparison of amount of protein secreted from cells transfected with the DNA plasmids on day 1 and day 3 after transfection, respectively. The results demonstrate that secretion of the proteins comprising a hCCL3L1 targeting unit with a D27A and/or E67A mutation which were expressed from cells transfected cells with MC38-I2-D27A, MC38-I2-E67A or MC38-I2-D27A-E67A on day 1 after transfection was markedly increased when compared to secretion of the protein comprising the wild type hCCL1L3 targeting unit on day 3 post transfection. In conclusion, proteins comprising the above-described mutated hCCL3L1 targeting units were not only secreted at higher amounts from transfected Expi293F cells, but also faster, compared to the protein comprising the wild type hCCL3L1 targeting unit.

### B. Assessment of Receptor Activation by Proteins Encoded by MC38-I2-WT, MC38-I2-D27A, MC38-I2-E67A and MC38-I2-D27A-E67A DNA Plasmids

The hCCR5 reporter assay was performed with cell supernatant of Example 6A from cells transfected with MC38-I2-WT, MC38-I2-D27A, MC38-I2-E67A and MC38-I2-D27A-E67A, using a commercially available kit (Thermo Fisher Scientific, K1788) according to the manufacturer's protocol adapted to a 96-well plate format (100 μL cell supernatant was added to 50 μL U2OS cell suspension) and as described in Example 1B. 3 nM recombinant CCL3L1 was used as a positive control. A sample of ExpiFectamine 293 reagent treated cells was included as a negative control.

The results presented in FIG. 31 demonstrate that the proteins encoded by MC38-I2-WT, MC38-I2-D27A, MC38-I2-E67A and MC38-I2-D27A-E67A bind to and activate the hCCR5 receptor.

C. Characterization of the Intact Proteins Expressed from MC38-I2-WT, MC38-I2-D27A, MC38-I2-E67A and MC38-I2-D27A-E67A DNA Plasmids

WB analysis was performed on supernatant samples from Example 6A to further characterize the proteins encoded by MC38-I2-WT, MC38-I2-D27A, MC38-I2-E67A and MC38-I2-D27A-E67A.

The samples were prepared, and the WB analysis was carried out as described in Example 4B.

The WB analysis confirmed the ELISA results of Example 6A, demonstrating that intact proteins were expressed from DNA plasmids MC38-I2-WT, MC38-I2-D27A, MC38-I2-E67A and MC38-I2-D27A-E67A (FIG. 32). In addition, and in line with the ELISA results, a stronger WB signal was detected for the proteins expressed from MC38-I2-D27A, MC38-I2-E67A and MC38-I2-D27A-E67A than for the protein expressed from MC38-I2-WT.

Taken together, the ELISA and western blot results demonstrate that intact proteins were expressed from DNA plasmids MC38-I2-WT, MC38-I2-D27A, MC38-I2-E67A and MC38-I 2-D27A-E67A and that the proteins were secreted from transfected Expi293F cells. The results further show that secretion of the proteins comprising a hCCL3L1 targeting unit with a D27A mutation and/or a D27A and a E67A mutation was accelerated and increased compared to that of the protein comprising a wild type hCCL3L1 targeting unit.

### Example 7

All DNA plasmids designed, produced and tested in Example 7 comprise nucleic acid sequences encoding the signal peptide and dimerization unit listed in Table 1 (but no nucleic acid sequence encoding the unit linker listed in said Table 1) and further comprise nucleic acid sequences encoding the targeting units listed in Table 11 below.

TABLE 11

| | Targeting unit |
|---|---|
| VB1026 | hCCL3L1 wild type |
| SEQ ID NO: 179 | SEQ ID NO: 2 |
| VB1026-D27A | hCCL3L1 with D27A mutation |
| SEQ ID NO: 180 | SEQ ID NO: 5 |
| VB1026-E67A | hCCL3L1 with E67A mutation |
| SEQ ID NO: 181 | SEQ ID NO: 6 |
| VB1026-D27A-E67A | hCCL3L1 with D27A and E67A mutations |
| SEQ ID NO: 182 | SEQ ID NO: 8 |
| VB1026-D27A-E67A-P8A | hCCL3L1 with D27A, E67A and P8A |
| SEQ ID NO: 183 | mutations |
| | SEQ ID NO: 141 |

DNA plasmids VB1026, VB1026-D27A, VB1026-E67A, VB1026-D27A-E67A and VB1026-D27A-E67A-P8A encode a protein comprising a hCCL3L1 targeting unit (wild type or comprising a D27A and/or E67A mutation or a D27A, E67A and P8A mutation), a dimerization unit but no unit linker and no antigenic unit.

### A. Assessment of Expression and Secretion of Proteins Encoded by VB1026, VB1026-D27A, VB1026-E67A and VB1026-D27A-E67A-P8A DNA Plasmids

Expi293F cells were seeded at a density of 2×10⁶ cells/mL and transfected with PEIpro (Polyplus) at 1 mL/L and 1.0 mg/L DNA. Cells were incubated with shaking at 37 °C for 7 days prior to supernatant harvesting. Protein purification from harvested supernatants was carried out using overnight incubation with FcXP resin (Thermo), followed by elution with 50 mM phosphoric acid buffer pH 3 and immediate neutralization with 20×PBS pH 11 and 1 M Tris pH 8 buffer to pH 7.5. Protein yield was assessed by UV absorption at 280 nm.

FIG. 33 shows the yields of purified proteins secreted from cells transfected with the DNA plasmids. The proteins encoded by VB1026, VB1026-D27A, VB1026-E67A and VB1026-D27A-E67A-P8A were secreted from transfected Expi293F cells and purified. The data further show that the proteins comprising D27A or E67A or D27A, E67A and P8A mutations resulted in higher yields than the protein comprising the wild type hCCL1L3 targeting unit.

### B. Assessment of Oligomerization State of the Purified Proteins

The purified proteins from Example 7A were assessed for their oligomerization state by size exclusion chromatography-multiple angle light scattering (SEC-MALS) (Wyatt Technology), (Waters XBridge BEH SEC 200A, 30.5 μm) using 100 mM Tris pH 7.5, 150 mM NaCl.

The size exclusion chromatograms presented in FIG. 34 show that inclusion of D27A or E67A or D27A, E67A and P8A mutations in the hCCL3L1 targeting unit reduces oligomerization in solution for the proteins comprising such mutations compared to the protein comprising the wild type hCCL3L1 targeting unit.

### C. Expression, Purification and Assessment of Oligomerization State of Proteins Encoded by VB1026, VB1026-D27A, VB1026-E67A, VB1026-D27A-E67A and VB1026-D27A-E67A-P8A DNA Plasmids

Expi293F cells were seeded, transfected, incubated and supernatant was harvested as described in Example 7A. Protein purification from harvested supernatants was carried out via overnight incubation with FcXP resin (Thermo), washing with PBS, elution with 50 mM Phosphoric acid pH3, and neutralization with 1 M Tris pH 7.5 (to 100 mM). Samples were dialyzed into 150 mm NaCl, 100 mM Tris pH 7.5 buffer using Amicon Ultra concentrators, 10K MWCO (Millipore). Purified proteins were assessed for their oligomerization state by size exclusion chromatography (SEC). Briefly, samples were analyzed over a TSKgelSuper SW3000 column (Tosoh Bioscience), with 200 mM potassium phosphate, 250 mM potassium chloride pH 7.0 running buffer, on a Thermo Vanquish (U)HPLC system. Oligomerization state was determined by observing US absorption at 280 nm and by comparing relative retention times.

The size exclusion chromatogram is presented in FIG. 35. Percent monomer versus oligomer for each sample was quantitatively determined by integrating the area under the curve for each sample. The percent monomer versus oligomer for each sample is shown in FIG. 36.

The SEC results and quantitative determination of percent monomer versus oligomer show that inclusion of D27A and/or E67A or D27A, E67A and P8A mutations in the hCCL3L1 targeting unit reduces oligomerization in solution for the proteins comprising such mutations compared to the protein comprising the wild type hCCL3L1 targeting unit.

### Example 8

All DNA plasmids designed, produced and tested in Example 8 comprise nucleic acid sequences encoding the elements/units listed in Table 1 and further comprise nucleic acid sequences encoding the targeting units and antigenic units listed in Table 12 below.

TABLE 12

| Element/ | mCherry-WT | mCherry-D27A-E67A-P8A |
|---|---|---|
| Units | SEQ ID NO: 184 | SEQ ID NO: 185 |
| Targeting | hCCL3L1 wild type | hCCL3L1 with D27A, E67A and |
| unit | SEQ ID NO: 2 | P8A mutation |
| | | SEQ ID NO: 141 |

| | |
|---|---|
| Antigenic | mCherry-Tev-His |
| unit | SEQ ID NO: 186 |

DNA plasmids mCherry-WT and mCherry-D27A-E67A-P8A comprise nucleic acid sequences encoding a hCCL3L1 targeting unit (wild type or with D27A, E67A and P8A mutations), a dimerization unit, and an antigenic unit comprising a fluorescent protein (mCherry) with a TEV cleavage consensus sequence and a histidine (H6) tag.

A. Assessment of Expression and Secretion of Proteins Encoded by mCherry-WT and mCherry-D27A-E67A-P8A DNA Plasmids

Expi293F cells were seeded, transfected, incubated and supernatant was harvested as described in Example 7A. Protein purification from harvested supernatants was carried out using an overnight incubation with anti-His resin (internal), followed by gravity column purification with 50 mM phosphoric acid elution buffer pH 3 and neutralization with 20×PBS pH 11. Fractions containing protein as determined by UV absorption at 280 nm were pooled and concentrated. Protein yield was assessed by UV absorption at 280 nm.

FIG. 37 shows the yields of purified proteins secreted from cells transfected with the DNA plasmids. The proteins encoded by mCherry-WT and mCherry-D27A-E67A-P8A were secreted from transfected Expi293F cells and purified. The data further show that the protein comprising the D27A, E67A and P8A mutations resulted in a higher yield than the protein comprising the wild type hCCL1L3 targeting unit.

### B. Assessment of Oligomerization State of Purified Proteins

The purified proteins from Example 8A were assessed for their oligomerization state by size exclusion chromatography as described in Example 7C.

The SEC results shown in FIGS. 38A (for mCherry-WT) and 38B (for mCherry-D27A-E67A-P8A) demonstrate that inclusion of D27A, E67A and P8A mutations in the hCCL3L1 targeting unit reduces oligomerization in solution for the protein encoded by mCherry-D27A-E67A-P8A compared to the protein encoded by mCherry-WT comprising the wild type hCCL3L1 targeting unit.

### Example 9

All DNA plasmids designed, produced and tested in Example 9 comprise nucleic acid sequences encoding the elements/units listed in Table 1. DNA plasmids MC38-I2-WT, MC38-I2-D27A, MC38-I2-E67A and MC38-I2-D27A-E67A further comprise nucleic acid sequences encoding the targeting units and antigenic units listed in Table 9, while DNA plasmid MC38-I2-D27A-E67A-P8A further comprises nucleic acid sequences encoding the targeting unit and antigenic unit listed in Table 13 below:

TABLE 13

| Elements/ | MC38-I2-D27A-E67A-P8A |
|---|---|
| Units | SEQ ID NO: 187 |
| Targeting | hCCL3L1 with D27A, E67A and P8A mutations |
| unit | SEQ ID NO: 141 |
| Antigenic | 20 MC38 neoantigens separated by glycine- |
| unit | serine linkers (see Table 10) |

### A. Assessment of Expression and Secretion of Proteins Encoded by MC38-I2-WT, MC38-I 2-D27A, MC38-I2-E67A, MC38-I2-D27A-E67A and MC38-I2-D27A-E67A-P8A DNA Plasmids

Mouse myoblast cells were engineered to express the protein encoded by each of the DNA plasmids under the control of a Tet-On doxycycline inducible promoter using the piggyBac system. Specifically, each of the constructs from Table 9 and 13 were cloned downstream of the TRE3G promoter in a vector containing the piggyBac transposon. To produce stably integrated cell lines expressing each construct, the plasmids were co-transfected with a plasmid encoding the piggyBac transposase at a ratio of 6:1 into mouse myoblast cells using lipofectamine 2000 following the manufacturer's protocol. 2.1 μg of total plasmid was transfected into a 6-well dish of mouse myoblast cells grown to about 50% confluence. Transfected cells were selected for stable integration and expression of each construct by adding puromycin to the media at a concentration of 1 μg/mL. Expression of the proteins was induced by growing cells in media supplemented with 1 μg/mL doxycycline.

The proteins present in the supernatants from cells expressing each of the stably integrated constructs were characterized in a sandwich ELISA using mouse anti-human IgG CH3 domain antibody (capture antibody, 100 μL/well, 1 μg/mL, MCA878G, Bio-Rad) and goat anti-human MIP-1α antibody (biotinylated detection antibody, 100 μL/well, 0.2 μg/mL, BAF270, R&D 10 systems). Supernatants were collected from cells grown for 72 hrs in media supplemented with 1 μg/mL of doxycycline to induce expression. The collected supernatants were diluted in a 5 point dose titration 1:1 with 1% BSA PBS. A sample of lipofectamine treated cells (transfection control) was included as a negative control.

FIG. 39 shows the amount of proteins secreted from cells which were stably transfected with the DNA plasmids. The proteins encoded by each of the different DNA plasmids were well expressed and secreted from stably transfected mouse myoblasts. The data further show that the proteins comprising the D27A and/or E67A mutation or the D27A, E67A and P8A in the hCCL3L1 targeting unit were secreted at higher amounts than the protein comprising the wild type hCCL1 L3 targeting unit.

### Example 10

The following DNA plasmids were designed, produced tested in Example 10: VB1026, VB1026-D27A, VB1026-D27A-E67A and VB1026-D27A-E67A-P8A as described in Example 7.

### A. Assessment of Expression and Secretion of Proteins Encoded by VB1026, VB1026-D27A, VB1026-D27A-E67A and VB1026-D27A-E67A-P8A DNA Plasmids

Mouse myoblast cells were engineered to express the protein encoded by each of the DNA plasmids as described in Example 9A and the proteins present in the supernatant from such cells were characterized in a sandwich ELISA as described in Example 9A.

FIG. 40 shows the amount of proteins secreted from cells which were stably transfected with the DNA plasmids. The proteins encoded by each of the different DNA plasmids were well expressed and secreted from stably transfected mouse myoblasts. The data further show that the proteins comprising the D27A, D27A and E67A or the D27A, E67A and P8A mutation in the hCCL3L1 targeting unit were secreted at markedly higher amounts than the protein comprising the wild type hCCL1 L3 targeting unit.

### B. Protein Purification and Assessment of Oligomerization State of Purified Proteins

Mouse myoblast cells stably integrated with each construct were grown for 72 hrs in 1 μg/mL doxycycline to induce protein expression and 30 mL of supernatant was harvested for each replicate. The supernatants were incubated with FcXP Affinity resin (Thermo cat. number A56249) for 1 hr at room temperature while shaking. The resin was washed, and the captured proteins eluted with 50 mM phosphoric acid pH 3 into 20×PBS pH 11 to neutralize. The yields of purified protein obtained (average of 3 transfection and purification experiments) are stated in Table 14.

TABLE 14

| | Yield (μg) |
|---|---|
| VB1026 | 94 |
| VB1026-D27A | 253 |
| VB1026-D27A-E67A | 213 |
| VB1026-D27A-E67A-P8A | 219 |

The purified proteins were assessed for oligomerization state by SEC. Samples were analyzed over a TSKGEL3000SW column (Tosoh Bioscience), with 100 mM Tris pH 7.5, 150 mM NaCl, on an Agilent 1200 HPLC system. Oligomerization state was determined by observing US absorption at 280 nm and by comparing relative retention times.

Table 14 shows the yields of purified proteins secreted from cells transfected with the DNA plasmids. The data show that hat inclusion of D27A, D27A and E67A or D27A, E67A and P8A mutations in the hCCL3L1 targeting unit results in higher protein purification yield for the proteins encoded by VB1026-D27A, VB1026-D27A-E67A and VB1026-D27A-E67A-P8A compared to the protein encoded by VB1026, comprising the wild type hCCL3L1 targeting unit.

The SEC results are presented in FIG. 41. Percent monomer versus oligomer for each sample was quantitatively determined by integrating the area under the curve for each sample. The percent monomer versus oligomer for each sample is shown in FIG. 42.

The SEC results and quantitative determination of percent monomer versus oligomer show that inclusion of D27A, D27A and E67A or D27A, E67A and P8A mutations in the hCCL3L1 targeting unit reduces oligomerization in solution for the proteins encoded by VB1026-D27A, VB1026-D27A-E67A and VB1026-D27A-E67A-P8A compared to the protein encoded by VB1026, comprising the wild type hCCL3L1 targeting unit.

### C. Mass Photometry

Mass photometry is a light scattering based technique that detects individual, unlabeled molecules in solution in their native state, providing information about the oligomerization state of such molecules.

A Two^{MP} Auto system and AcquireMP Software (Refeyn) was used to conduct the mass photometry experiments. BSA and Herceptin were included as calibrants for molecular weight determination. Samples of purified proteins obtained from Example 10B were diluted to 10 nM in freshly made, sterile filtered PBS, pH 7.4. For data acquisition, 14 well cassettes over glass coverslips were used. First 10 μL of PBS was added to each well to allow for camera focusing, followed by addition of 10 μL of sample and mixing, giving a final sample concentration of 5 nM. Videos of one minute in length were obtained and data were analyzed using DiscoverMP software (Refeyn). Contrast data obtained from each video were converted to molecular weight using the calibration function with the curve obtained from BSA and Herceptin standards. Data were plotted as both histograms and kernel density estimate (KDE) distributions, and peaks were fit with Gaussian functions to obtain the average mass of each component.

The results are shown in FIGS. 43A-D, the monomer is the peak at about 50 kDa, while the peaks with higher molecular weight are the oligomers. The results from the mass photometry are aligned with those obtained from SEC analysis, i.e. they show that inclusion of D27A (FIG. 43B), D27A and E67A (FIG. 43C) or D27A, E67A and P8A mutations (FIG. 43D) in the hCCL3L1 targeting unit reduces oligomerization in solution for the proteins encoded by VB1026-D27A, VB1026-D27A-E67A and VB1026-D27A-E67A-P8A compared to the protein encoded by VB1026, comprising the wild type hCCL3L1 targeting unit (FIG. 43A).

Sequence overview
SEQ ID NO: 1
ASLAADTPTACCFSYTSRQIPQNFIADYFETSSQCSKPGVIFLTKRSRQVCADPSEEW
VQKYVSDLELSA
SEQ ID NO: 2
APLAADTPTACCFSYTSRQIPQNFIADYFETSSQCSKPSVIFLTKRGRQVCADPSEEW
VQKYVSDLELSA
SEQ ID NO: 3
ASLAADTPTACCFSYTSRQIPQNFIAAYFETSSQCSKPGVIFLTKRSRQVCADPSEEW
VQKYVSDLELSA
SEQ ID NO: 4
ASLAADTPTACCFSYTSRQIPQNFIADYFETSSQCSKPGVIFLTKRSRQVCADPSEEW
VQKYVSDLALSA
SEQ ID NO: 5
APLAADTPTACCFSYTSRQIPQNFIAAYFETSSQCSKPSVIFLTKRGRQVCADPSEEW
VQKYVSDLELSA
SEQ ID NO: 6
APLAADTPTACCFSYTSRQIPQNFIADYFETSSQCSKPSVIFLTKRGRQVCADPSEEW
VQKYVSDLALSA
SEQ ID NO: 7
ASLAADTPTACCFSYTSRQIPQNFIAAYFETSSQCSKPGVIFLTKRSRQVCADPSEEW
VQKYVSDLALSA
SEQ ID NO: 8
APLAADTPTACCFSYTSRQIPQNFIAAYFETSSQCSKPSVIFLTKRGRQVCADPSEEW
VQKYVSDLALSA
SEQ ID NO: 140
ASLAADTATACCFSYTSRQIPQNFIAAYFETSSQCSKPGVIFLTKRSRQVCADPSEEW
VQKYVSDLALSA
SEQ ID NO: 141
APLAADTATACCFSYTSRQIPQNFIAAYFETSSQCSKPSVIFLTKRGRQVCADPSEEW
VQKYVSDLALSA
SEQ ID NO: 116
MQVSTAALAVLLCTMALCNQFS
SEQ ID NO: 117
MQVSTAALAVLLCTMALCNQVLS
SEQ ID NO: 18
E1LKTPLGDTTHT¹²E¹³PKSCDTPPPCPRCP²⁷G²⁸GGSSGGGSG³⁷G³⁸QPREPQVYTLP
PSREEMTKNQVSLTCLVKGFYPSDIAVEWESSGQPENNYNTTPPMLDSDGSFFLYSK
LTVDKSRWQQGNIFSCSVMHEALHNRFTQKSLSLSPGK¹⁴⁴
SEQ ID NO: 51
GLGGL
VB1020
SEQ ID NO: 118
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQL
TECH011-CV006
SEQ ID NO: 119
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQL
SEQ ID NO: 120
MHGDTPTLHEYMLDLQPETTDLYGYGQLNDSSEEEDEIDGPAGQAEPDRAHYNIVTF
CCKCDSTLRLCVQSTHVDIRTLEDLLMGTLGIVCPICSQKPGGGSSGGGSGMFQDPQ
ERPRKLPQLCTELQTTIHDIILECVYCKQQLLRREVYDFARRDLCIVYRDGNPYAVRDK
CLKFYSKISEYRHYCYSLYGTTLEQQYNKPLCDLLIRCINRQKPLCPEEKQRHLDKKQ
RFHNIRGRWTGRCMSCCRSSRTRRETQL
VB2060
SEQ ID NO: 121
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNF
VB4231
SEQ DI NO: 122
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNFEPEA
TECH011-IV002
SEQ ID NO: 123
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNF
TECH011-IV011
SEQ ID NO: 124
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNFEPEA
SEQ ID NO: 125
RVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNF
SEQ ID NO: 126
EPEA
VB4097
SEQ ID NO: 127
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKIYEFDYHLYGQNITMIMTSVSGHLLAGGGGSGGGGSAEYGDY
QPEVHGVPYFRLEHYLPARVMGGGGSGGGGSGSLFGSSRVQYVVNPAVKIVFLNID
PSGGGGSGGGGSLWVYLRPVPRPATIYLQILRLKPLTGEGGGGSGGGGSTLAFLVLS
TPAMFNRALKPFLKSCHFQGGGGSGGGGSFVSPMAHYVPGIMAIESVVARFQFIVPG
GGGSGGGGSVILPQAPSGPSYATYLQPAQAQMLTPPGGGGGGGGSEVIQTSKYY
MRDVIAIESAWLLELAPHGGGGSGGGGSDTLSAMSNPRAMQVLLQIQQGLQTLATG
GGGSGGGGSKSWIHCWKYLSVQSQLFRGSSLLFRRV
TECH011-CV007
SEQ ID NO: 128
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLKIYEFDYHLYGQNITMIMTSVSGHLLAGGGGGGGGSAEYGDY
QPEVHGVPYFRLEHYLPARVMGGGGGGGGSGSLFGSSRVQYVVNPAVKIVFLNID
PSGGGGSGGGGSLWVYLRPVPRPATIYLQILRLKPLTGEGGGGSGGGGSTLAFLVLS
TPAMFNRALKPFLKSCHFQGGGGSGGGGSFVSPMAHYVPGIMAIESVVARFQFIVPG
GGGSGGGGSVILPQAPSGPSYATYLQPAQAQMLTPPGGGGSGGGGSEVIQTSKYY
MRDVIAIESAWLLELAPHGGGGSGGGGSDTLSAMSNPRAMQVLLQIQQGLQTLATG
GGGSGGGGSKSWIHCWKYLSVQSQLFRGSSLLFRRV
SEQ ID NO: 139
GCACCACTTGCTGCTGACACGCCGACCGCCTGCTGCTTCAGCTACACCTCCCGA
CAGATTCCACAGAATTTCATAGCTGCTTACTTTGAGACGAGCAGCCAGTGCTCCAA
GCCCAGTGTCATCTTCCTAACCAAGAGAGGCCGGCAGGTCTGTGCTGACCCCAG
TGAGGAGTGGGTCCAGAAATACGTCAGTGACCTGGAGCTGAGTGCC
TECH011-CV036
SEQ ID NO: 142
MQVSTAALAVLLCTMALCNQVLSAPLAADTPTACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLMHGDTPTLHEYMLDLQPETTDLYGYGQLNDSSEEEDEIDGPAG
QAEPDRAHYNIVTFCCKCDSTLRLCVQSTHVDIRTLEDLLMGTLGIVCPICSQKPGGG
SSGGGSGMFQDPQERPRKLPQLCTELQTTIHDIILECVYCKQQLLRREVYDFARRDLC
IVYRDGNPYAVRDKCLKFYSKISEYRHYCYSLYGTTLEQQYNKPLCDLLIRCINRQKPL
CPEEKQRHLDKKQRFHNIRGRWTGRCMSCCRSSRTRRETQL
TECH011-IV015
SEQ ID NO: 154
MQVSTAALAVLLCTMALCNQVLSAPLAADTPTACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVK
NKCVNF
MC38-12-WT
SEQ ID NO: 155
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLVDFTGSNGDPSSPYSLHYLSPTGVNEYGGGGSGGGGSGEKRS
ISAIRSYQYVMKICAAHLPTESGGGGSGGGGSYMVRFKDTKAQFSFFNPKCLLPTSR
HYGGGGSGGGGSAEPESPEKEQAYLGYLAMLEELKKQEAGGGGSGGGGSESEGLH
EVLAFAILKSDMDLRRTLFSNGGGGSGGGGSSKLLSFMAPIDHTTMSDDARTELFRSL
GGGGSGGGGSSMDIDPSSSVLFEYMEKPDFSLFSPTMGGGGSGGGGSSLVISASIIV
FNLLELEGDYRDDHIFSGGGGSGGGGSNGRVLELFRAAQLANDVVLQIMELCGAGG
GGSGGGGSKARDETAALLNSAVLGAAPLFVPPADCGGGGSGGGGSLSNRILWIGIAN
FQLCPLILIWQILYAGGGGSGGGGSGIPVHLELASMTNMELMSSIVHQQVFPGGGGS
GGGGSTLPLQPFQLAFGHLVNRQVFRQGPQPSGGGGSGGGGSITTDVLYTICNPCV
PVQRIVIFRKNGVGGGGSGGGGSHVLPKCKHEFCTSSISKAMLIKPVCPVGGGGSGG
GGSRGGGLLKYCHLLVLGFRPRPSTDVRALGGGGSGGGGSRILKAGGKILTFDRLAL
ESPKGRGTVLGGGGSGGGGSNNRVAVATINFRRLVCPQEDKTSTDVLGGGGSGGG
GSVCKACDKSFHFYCPLKVHMKRCRVAKSGGGGSGGGGSSSSAYTGYVERSPLAA
STYSLDILYSG
MC38-12-D27A
SEQ ID NO: 156
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLVDFTGSNGDPSSPYSLHYLSPTGVNEYGGGGSGGGGSGEKRS
ISAIRSYQYVMKICAAHLPTESGGGGSGGGGSYMVRFKDTKAQFSFFNPKCLLPTSR
HYGGGGSGGGGSAEPESPEKEQAYLGYLAMLEELKKQEAGGGGSGGGGSESEGLH
EVLAFAILKSDMDLRRTLFSNGGGGSGGGGSSKLLSFMAPIDHTTMSDDARTELFRSL
GGGGSGGGGSSMDIDPSSSVLFEYMEKPDFSLFSPTMGGGGSGGGGSSLVISASIIV
FNLLELEGDYRDDHIFSGGGGSGGGGSNGRVLELFRAAQLANDVVLQIMELCGAGG
GGSGGGGSKARDETAALLNSAVLGAAPLFVPPADCGGGGSGGGGSLSNRILWIGIAN
FQLCPLILIWQILYAGGGGGGGGSGIPVHLELASMTNMELMSSIVHQQVFPGGGGS
GGGGSTLPLQPFQLAFGHLVNRQVFRQGPQPSGGGGSGGGGSITTDVLYTICNPCV
PVQRIVIFRKNGVGGGGSGGGGSHVLPKCKHEFCTSSISKAMLIKPVCPVGGGGSGG
GGSRGGGLLKYCHLLVLGFRPRPSTDVRALGGGGSGGGGSRILKAGGKILTFDRLAL
ESPKGRGTVLGGGGSGGGGSNNRVAVATINFRRLVCPQEDKTSTDVLGGGGSGGG
GSVCKACDKSFHFYCPLKVHMKRCRVAKSGGGGSGGGGSSSSAYTGYVERSPLAA
STYSLDILYSG
MC38-12-E67A
SEQ ID NO: 157
MQVSTAALAVLLCTMALCNQVLSAPLAADTPTACCFSYTSRQIPQNFIADYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLVDFTGSNGDPSSPYSLHYLSPTGVNEYGGGGSGGGGSGEKRS
ISAIRSYQYVMKICAAHLPTESGGGGSGGGGSYMVRFKDTKAQFSFFNPKCLLPTSR
HYGGGGSGGGGSAEPESPEKEQAYLGYLAMLEELKKQEAGGGGSGGGGSESEGLH
EVLAFAILKSDMDLRRTLFSNGGGGGGGGSSKLLSFMAPIDHTTMSDDARTELFRSL
GGGGSGGGGSSMDIDPSSSVLFEYMEKPDFSLFSPTMGGGGSGGGGSSLVISASIIV
FNLLELEGDYRDDHIFSGGGGSGGGGSNGRVLELFRAAQLANDVVLQIMELCGAGG
GGSGGGGSKARDETAALLNSAVLGAAPLFVPPADCGGGGSGGGGSLSNRILWIGIAN
FQLCPLILIWQILYAGGGGSGGGGSGIPVHLELASMTNMELMSSIVHQQVFPGGGGS
GGGGSTLPLQPFQLAFGHLVNRQVFRQGPQPSGGGGSGGGGSITTDVLYTICNPCV
PVQRIVIFRKNGVGGGGSGGGGSHVLPKCKHEFCTSSISKAMLIKPVCPVGGGGSGG
GGSRGGGLLKYCHLLVLGFRPRPSTDVRALGGGGSGGGGSRILKAGGKILTFDRLAL
ESPKGRGTVLGGGGSGGGGSNNRVAVATINFRRLVCPQEDKTSTDVLGGGGSGGG
GSVCKACDKSFHFYCPLKVHMKRCRVAKSGGGGSGGGGSSSSAYTGYVERSPLAA
STYSLDILYSG
MC38-12-D27A-E67A
SEQ ID NO: 158
MQVSTAALAVLLCTMALCNQVLSAPLAADTPTACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLVDFTGSNGDPSSPYSLHYLSPTGVNEYGGGGSGGGGSGEKRS
ISAIRSYQYVMKICAAHLPTESGGGGSGGGGSYMVRFKDTKAQFSFFNPKCLLPTSR
HYGGGGSGGGGSAEPESPEKEQAYLGYLAMLEELKKQEAGGGGSGGGGSESEGLH
EVLAFAILKSDMDLRRTLFSNGGGGSGGGGSSKLLSFMAPIDHTTMSDDARTELFRSL
GGGGSGGGGSSMDIDPSSSVLFEYMEKPDFSLFSPTMGGGGSGGGGSSLVISASIIV
FNLLELEGDYRDDHIFSGGGGSGGGGSNGRVLELFRAAQLANDVVLQIMELCGAGG
GGSGGGGSKARDETAALLNSAVLGAAPLFVPPADCGGGGSGGGGSLSNRILWIGIAN
FQLCPLILIWQILYAGGGGSGGGGSGIPVHLELASMTNMELMSSIVHQQVFPGGGGS
GGGGSTLPLQPFQLAFGHLVNRQVFRQGPQPSGGGGSGGGGSITTDVLYTICNPCV
PVQRIVIFRKNGVGGGGSGGGGSHVLPKCKHEFCTSSISKAMLIKPVCPVGGGGSGG
GGSRGGGLLKYCHLLVLGFRPRPSTDVRALGGGGSGGGGSRILKAGGKILTFDRLAL
ESPKGRGTVLGGGGSGGGGSNNRVAVATINFRRLVCPQEDKTSTDVLGGGGSGGG
GSVCKACDKSFHFYCPLKVHMKRCRVAKSGGGGSGGGGSSSSAYTGYVERSPLAA
STYSLDILYSG
VB1026
SEQ ID NO: 179
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGK
VB1026-D27A
SEQ ID NO: 180
MQVSTAALAVLLCTMALCNQVLSAPLAADTPTACCFSYTSRQIPQNFIAAYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGK
VB1026-E67A
SEQ ID NO: 181
MQVSTAALAVLLCTMALCNQVLSAPLAADTPTACCFSYTSRQIPQNFIADYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGK
VB1026-D27A-E67A
SEQ ID NO: 182
MQVSTAALAVLLCTMALCNQVLSAPLAADTPTACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGK
VB1026-D27A-E67A-P8A
SEQ ID NO: 183
MQVSTAALAVLLCTMALCNQVLSAPLAADTATACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGK
mCherry-WT
SEQ ID NO: 184
MQVSTAALAVLLCTMALCNQVLSAPLAADTPTACCFSYTSRQIPQNFIADYFETSSQC
SKPSVIFLTKRGRQVCADPSEEWVQKYVSDLELSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLENLYFQSMLSKGEEDNMAIIKEFMRFKVHMEGSVNGHEFEIEG
EGEGRPYEGTQTAKLKVTKGGPLPFAWDILSPQFMYGSKAYVKHPADIPDYLKLSFPE
GFKWERVMNFEDGGVVTVTQDSSLQDGEFIYKVKLRGTNFPSDGPVMQKKTMGWE
ASSERMYPEDGALKGEIKQRLKLKDGGHYDAEVKTTYKAKKPVQLPGAYNVNIKLDIT
SHNEDYTIVEQYERAEGRHSTGGMDELYKHHHHHH
mCherry-D27A-E67A-P8A
SEQ ID NO: 185
MQVSTAALAVLLCTMALCNQVLSAPLAADTATACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLENLYFQSMLSKGEEDNMAIIKEFMRFKVHMEGSVNGHEFEIEG
EGEGRPYEGTQTAKLKVTKGGPLPFAWDILSPQFMYGSKAYVKHPADIPDYLKLSFPE
GFKWERVMNFEDGGVVTVTQDSSLQDGEFIYKVKLRGTNFPSDGPVMQKKTMGWE
ASSERMYPEDGALKGEIKQRLKLKDGGHYDAEVKTTYKAKKPVQLPGAYNVNIKLDIT
SHNEDYTIVEQYERAEGRHSTGGMDELYKHHHHHH
SEQ ID NO: 186
ENLYFQSMLSKGEEDNMAIIKEFMRFKVHMEGSVNGHEFEIEGEGEGRPYEGTQTAK
LKVTKGGPLPFAWDILSPQFMYGSKAYVKHPADIPDYLKLSFPEGFKWERVMNFEDG
GVVTVTQDSSLQDGEFIYKVKLRGTNFPSDGPVMQKKTMGWEASSERMYPEDGALK
GEIKQRLKLKDGGHYDAEVKTTYKAKKPVQLPGAYNVNIKLDITSHNEDYTIVEQYERA
EGRHSTGGMDELYKHHHHHH
MC38-12-D27A-E67A-P8A
SEQ ID NO: 187
MQVSTAALAVLLCTMALCNQVLSAPLAADTATACCFSYTSRQIPQNFIAAYFETSSQC
SKPSVIFLTKRGRQVCADPSEEWVQKYVSDLALSAELKTPLGDTTHTEPKSCDTPPPC
PRCPGGGSSGGGSGGQPREPQVYTLPPSREEMTKNQVSLTCLVKGFYPSDIAVEWE
SSGQPENNYNTTPPMLDSDGSFFLYSKLTVDKSRWQQGNIFSCSVMHEALHNRFTQ
KSLSLSPGKGLGGLVDFTGSNGDPSSPYSLHYLSPTGVNEYGGGGSGGGGSGEKRS
ISAIRSYQYVMKICAAHLPTESGGGGSGGGGSYMVRFKDTKAQFSFFNPKCLLPTSR
HYGGGGSGGGGSAEPESPEKEQAYLGYLAMLEELKKQEAGGGGSGGGGSESEGLH
EVLAFAILKSDMDLRRTLFSNGGGGSGGGGSSKLLSFMAPIDHTTMSDDARTELFRSL
GGGGSGGGGSSMDIDPSSSVLFEYMEKPDFSLFSPTMGGGGSGGGGSSLVISASIIV
FNLLELEGDYRDDHIFSGGGGSGGGGSNGRVLELFRAAQLANDVVLQIMELCGAGG
GGSGGGGSKARDETAALLNSAVLGAAPLFVPPADCGGGGSGGGGSLSNRILWIGIAN
FQLCPLILIWQILYAGGGGSGGGGSGIPVHLELASMTNMELMSSIVHQQVFPGGGGS
GGGGSTLPLQPFQLAFGHLVNRQVFRQGPQPSGGGGSGGGGSITTDVLYTICNPCV
PVQRIVIFRKNGVGGGGSGGGGSHVLPKCKHEFCTSSISKAMLIKPVCPVGGGGSGG
GGSRGGGLLKYCHLLVLGFRPRPSTDVRALGGGGSGGGGSRILKAGGKILTFDRLAL
ESPKGRGTVLGGGGSGGGGSNNRVAVATINFRRLVCPQEDKTSTDVLGGGGSGGG
GSVCKACDKSFHFYCPLKVHMKRCRVAKSGGGGSGGGGSSSSAYTGYVERSPLAA
STYSLDILYSG
SEQ ID NO: 188
GCACCACTTGCTGCTGACACGCCGACCGCCTGCTGCTTCAGCTACACCTCCCGA
CAGATTCCACAGAATTTCATAGCTGACTACTTTGAGACGAGCAGCCAGTGCTCCA
AGCCCAGTGTCATCTTCCTAACCAAGAGAGGCCGGCAGGTCTGTGCTGACCCCA
GTGAGGAGTGGGTCCAGAAATACGTCAGTGACCTGGCCCTGAGTGCC
SEQ ID NO: 189
GCACCACTTGCTGCTGACACGCCGACCGCCTGCTGCTTCAGCTACACCTCCCGA
CAGATTCCACAGAATTTCATAGCTGCTTACTTTGAGACGAGCAGCCAGTGCTCCAA
GCCCAGTGTCATCTTCCTAACCAAGAGAGGCCGGCAGGTCTGTGCTGACCCCAG
TGAGGAGTGGGTCCAGAAATACGTCAGTGACCTGGCCCTGAGTGCC
SEQ ID NO: 190
GCACCACTTGCTGCTGACACGGCCACCGCCTGCTGCTTCAGCTACACCTCCCGA
CAGATTCCACAGAATTTCATAGCTGCTTACTTTGAGACGAGCAGCCAGTGCTCCAA
GCCCAGTGTCATCTTCCTAACCAAGAGAGGCCGGCAGGTCTGTGCTGACCCCAG
TGAGGAGTGGGTCCAGAAATACGTCAGTGACCTGGCCCTGAGTGCC

### EMBODIMENTS

1. A construct, the construct being:
(i) a polynucleotide comprising a nucleotide sequence encoding a targeting unit selected from the group consisting of mutated human CCL3 (hCCL3) and mutated human CCL3L1 (hCCL3L1) and an antigenic unit comprising one or more antigens or parts thereof; or
(ii) a polypeptide encoded by the nucleotide sequence as defined in (i),
wherein the amino acid sequence of the targeting unit comprises one or more mutations, compared to the amino acid sequence of the wild type, which reduce or prevent the formation of targeting unit oligomers.

2. A construct, the construct being:
(i) a polypeptide comprising a targeting unit selected from the group consisting of mutated human CCL3 (hCCL3) and mutated human CCL3L1 (hCCL3L1) and an antigenic unit comprising one or more antigens or parts thereof, wherein the targeting unit comprises one or more mutations, compared to the wild type, which reduce or prevent the formation of targeting unit oligomers; or
(ii) a polynucleotide encoding the polypeptide as defined in (i).

3. The construct as described in embodiment 1 or 2, wherein the nucleotide sequence further encodes a multimerization unit, such as a dimerization unit or wherein the polypeptide further comprises a multimerization unit, such as a dimerization unit.

4. The construct as described in any of embodiments 1 to 3, wherein the one or more mutations reduce or prevent the formation of targeting unit oligomers in solution.

5. The construct as described in embodiment 4, wherein the one or more mutations reduce or prevent the formation of targeting unit oligomers in solution under relevant physiological conditions.

6. The construct as described in any of embodiments 1 to 5, wherein the mutation is a substitution of an amino acid comprised in the wild type amino acid sequence with a different amino acid.

7. The construct as described in any of embodiments 1 to 6, wherein the mutation does not lead to a decreased solubility and/or decreased ability of receptor binding and/or decreased ability of receptor activation of the mutated hCCL3 or hCCL3L1 targeting unit, compared to the solubility and/or ability of receptor binding and/or ability of receptor activation of the hCCL3 or hCCL3L1 wild type targeting unit.

8. The construct as described in any of embodiments 1 to 7, wherein the mutation is designed to disrupt the salt bridge formed by amino acid residues D27 and R46 in crystalized polymers formed by the interaction of hCCL3 wild type monomers or hCCL3L1 wild type monomers.

9. The construct as described in embodiment 8, wherein the amino acid D27 of the wild type amino acid sequence of hCCL3 or hCCL3L1 is substituted with a small, non-polar amino acid, such as an amino acid selected from the group consisting of G, A, V, L and 1.

10. The construct as described in embodiment 8, wherein the amino acid D27 of the wild type amino acid sequence of hCCL3 or hCCL3L1 is substituted with an amino acid is selected from the group consisting of A, S and Q.

11. The construct as described in embodiment 8, wherein the amino acid D27 of the wild type amino acid sequence of hCCL3 or hCCL3L1 is substituted with alanine.

12. The construct as described in any of embodiments 1 to 7, wherein the mutation is designed to disrupt the salt bridge formed by amino acid residues E67 and R48 in crystalized polymers formed by the interaction of hCCL3 wild type monomers or hCCL3L1 wild type monomers.

13. The construct as described in embodiment 12, wherein the amino acid E67 of the wild type amino acid sequence of hCCL3 or hCCL3L1 is substituted with a small, non-polar amino acid, such as an amino acid selected from the group consisting of G, A, V, L and I.

14. The construct as described in embodiment 12, wherein the amino acid E67 of the wild type amino acid sequence of hCCL3 or hCCL3L1 is substituted with an amino acid is selected from the group consisting of A, S and Q.

15. The construct as described in embodiment 12, wherein the amino acid E67 of the wild type amino acid sequence of hCCL3 or hCCL3L1 is substituted with alanine.

16. The construct as described in any of embodiments 1 to 7, wherein the mutation is designed to disrupt the hydrogen bond between amino acid residues D6 and S33 in crystalized polymers formed by the interaction of hCCL3 wild type monomers or hCCL3L1 wild type monomers.

17. The construct as described in embodiment 16, wherein the amino acid D6 of the wild type amino acid sequence of hCCL3 or hCCL3L1 is substituted with a small, non-polar amino acid, such as an amino acid selected from the group consisting of G, A, V, L and I.

18. The construct as described in embodiment 16, wherein the amino acid D6 of the wild type amino acid sequence of hCCL3 or hCCL3L1 is substituted with alanine.

19. The construct as described in any of embodiments 1 to 7, wherein the mutation is designed to disrupt the hydrophobic interactions of amino acid residue F24 in crystalized polymers formed by the interaction of hCCL3 wild type monomers or hCCL3L1 wild type monomers.

20. The construct as described in embodiment 19, wherein the amino acid F24 of the wild type amino acid sequence of hCCL3 or hCCL3L1 is substituted with a small, non-polar amino acid, such as an amino acid selected from the group consisting of G, A, V, L and I.

21. The construct as described in embodiment 19, wherein the amino acid F24 of the wild type amino acid sequence of hCCL3 or hCCL3L1 is substituted with alanine.

22. The construct as described in any of embodiments 1 to 7, wherein the mutation is designed to disrupt the hydrophobic interactions of amino acid residue F29 in crystalized polymers formed by the interaction of hCCL3 wild type monomers or hCCL3L1 wild type monomers.

23. The construct as described in embodiment 22, wherein the amino acid F29 of the wild type amino acid sequence of hCCL3 or hCCL3L1 is substituted with a small, non-polar amino acid, such as an amino acid selected from the group consisting of G, A, V, L and I.

24. The construct as described in embodiment 22, wherein the amino acid F29 of the wild type amino acid sequence of hCCL3 or hCCL3L1 is substituted with alanine.

25. The construct as described in any of embodiments 1 to 7, wherein the mutation is designed to disrupt the hydrophobic interactions of amino acid residue Y28 in crystalized polymers formed by the interaction of hCCL3 wild type monomers or hCCL3L1 wild type monomers.

26. The construct as described in embodiment 25, wherein the amino acid Y28 of the wild type amino acid sequence of hCCL3 or hCCL3L1 is substituted with a small, non-polar amino acid, such as an amino acid selected from the group consisting of G, A, V, L and I.

27. The construct as described in embodiment 25, wherein the amino acid Y28 of the wild type amino acid sequence of hCCL3 or hCCL3L1 is substituted with alanine.

28. The construct as described in any of embodiments 1 to 7, wherein the amino acid P8 of the wild type amino acid sequence of hCCL3 or hCCL3L1 is substituted with a non-polar amino acid, such as a small, non-polar amino acid.

29. The construct as described in embodiment 28, wherein the amino acid P8 of the wild type amino acid sequence of hCCL3 or hCCL3L1 is substituted with alanine.

30. The construct as described in any of embodiments 1 to 7, wherein the targeting unit comprises a D27A mutation.

31. The construct as described in any of embodiments 1 to 7, wherein the targeting unit comprises an E67A mutation.

32. The construct as described in any of embodiments 1 to 7, wherein the targeting unit comprises a D6A mutation.

33. The construct as described in any of embodiments 1 to 7, wherein the targeting unit comprises a F24A mutation.

34. The construct as described in any of embodiments 1 to 7, wherein the targeting unit comprises a F29A mutation.

35. The construct as described in any of embodiments 1 to 7, wherein the targeting unit comprises a Y28A mutation.

36. The construct as described in any of embodiments 1 to 7, wherein the targeting unit comprises a P8A mutation.

37. The construct as described in any of embodiments 1 to7, wherein the targeting unit comprises several mutations selected from the group consisting of D27A, E67A, D6A, F24A, F29A, Y28A and P8A.

38. The construct as described in embodiment 36, wherein the targeting unit comprises a D27A mutation and an E67A mutation.

39. The construct as described in embodiment 36, wherein the targeting unit comprises a D27A mutation, an E67A mutation and a P8A mutation.

40. The construct as described in any of embodiments 1 to 39, wherein the targeting unit is mutated hCCL3L1.

41. The construct as described in any of embodiments 1 to 39, wherein the targeting unit is mutated hCCL3.

42. The construct as described in embodiment 40, wherein the targeting unit comprises an amino acid sequence selected from the group consisting of SEQ ID NO: 5, SEQ ID NO: 6, SEQ ID NO: 8, SEQ ID NO: 141, amino acid 3-70 of SEQ ID NO: 5, amino acid 3-70 of SEQ ID NO: 6, amino acid 3-70 of SEQ ID NO: 8 and amino acid 3-70 of SEQ ID NO: 141.

43. The construct as described in embodiment 40, wherein the targeting unit consists of the amino acid sequence selected from the group consisting of SEQ ID NO: 5, SEQ ID NO: 6, SEQ ID NO: 8, SEQ ID NO: 141, amino acid 3-70 of SEQ ID NO: 5, amino acid 3-70 of SEQ ID NO: 6, amino acid 3-70 of SEQ ID NO: 8 and amino acid 3-70 of SEQ ID NO: 141.

44. The construct as described in embodiment 40, wherein the targeting unit comprises the amino acid sequence of SEQ ID NO: 5 or comprises the amino acid sequence 3-70 of SEQ ID NO: 5.

45. The construct as described in embodiment 40, wherein the targeting unit consists of the amino acid sequence of SEQ ID NO: 5 or consist of the amino acid sequence 3-70 of SEQ ID NO: 5.

46. The construct as described in embodiment 40, wherein the construct is the polynucleotide comprising a nucleotide sequence selected from the group consisting of SEQ ID NO: 139, SEQ ID NO: 188, SEQ ID NO: 189 and SEQ ID NO: 190, which encodes the targeting unit.

47. The construct as described in embodiment 41, wherein the targeting unit comprises an amino acid sequence selected from the group consisting of SEQ ID NO: 3, SEQ ID NO: 4, SEQ ID NO: 7 and SEQ ID NO: 140.

48. The construct as described in embodiment 41, wherein the targeting unit consists of the amino acid sequence selected from the group consisting of SEQ ID NO: 3, SEQ ID NO: 4, SEQ ID NO: 7 and SEQ ID NO: 140.

49. The construct as described in embodiment 41, wherein the targeting unit comprises the amino acid sequence of SEQ ID NO: 3

50. The construct as described in embodiment 41, wherein the targeting unit consists of the amino acid sequence of SEQ ID NO: 3.

51. The construct according to any of embodiments 2 to 50, wherein the multimerization unit is selected from the group consisting of dimerization unit, trimerization unit and tetramerization unit and wherein said multimerization unit optionally comprises a hinge region which has the ability to form one or more covalent bonds.

52. The construct according to embodiment 51, wherein the multimerization unit is a trimerization unit, such as a human collagen-derived trimerization or the C-terminal domain of T4 fibritin.

53. The construct according to embodiment 52, wherein the multimerization unit is a human collagen-derived trimerization unit, preferably one selected from the group consisting of human collagen derived XVIII-derived trimerization domain and human collagen XV-derived trimerization domain.

54. The construct according to embodiment 51, wherein the multimerization unit is a tetramerization unit which is a domain derived from p53.

55. The construct according to any of embodiments 51 to 54, wherein the multimerization unit comprises a hinge region which has the ability to form one or more covalent bonds.

56. The construct according to any of embodiments 51 to 55, wherein the hinge region is Ig derived, such as derived from human Ig, such as derived from hIgG1 or hIgG2 or hIgG3 or from hIgM.

57. The construct according to any of embodiments 51 and 55 to 56, wherein the multimerization unit is a dimerization unit and said dimerization unit further comprises another domain that facilitates dimerization.

58. The construct according to embodiment 57, wherein the other domain is an immunoglobulin domain, preferably an immunoglobulin constant domain.

59. The construct according to any of embodiments 57 to 58, wherein the other domain is a carboxyterminal C domain derived from IgG, preferably from IgG3, more preferably from hIgG3.

60. The construct according to any of embodiments 57 to 59, wherein the dimerization unit further comprises a dimerization unit linker, such as glycine-serine rich linker, such as GGGSSGGGSG.

61. The construct according to embodiment 60, wherein the dimerization unit linker connects the hinge region and the other domain that facilitates dimerization.

62. The construct according to any of embodiments 57 to 61, wherein the dimerization unit comprises hinge exon h1 and hinge exon h4, a dimerization unit linker and a CH3 domain of human IgG3.

63. The construct according to embodiment 62, wherein the dimerization unit comprises an amino acid sequence having at least 80% sequence identity to the amino acid sequence of SEQ ID NO: 18.

64. The construct according to embodiment 63, wherein the dimerization unit consists of an amino acid sequence having at least 80% sequence identity to the amino acid sequence of SEQ ID NO: 18.

65. The construct according to embodiment 64, wherein the dimerization unit consists of the amino acid sequence of SEQ ID NO: 18.

66. The construct according to any of embodiments 1 to 65, wherein the antigenic unit comprises one or more disease-relevant antigens or parts thereof.

67. The construct according to any of embodiments 1 to 66, wherein the antigenic unit comprises one or more neoantigens or parts thereof.

68. The construct according to embodiment 67, wherein the antigenic unit comprises one or more parts of one or more neoantigens.

69. The construct according to embodiment 68, wherein said parts are neoepitopes.

70. The construct according to embodiment 69, wherein the antigenic unit comprises several neoepitopes, such as several neoepitopes which are separated from each other by linkers.

71. The construct according to any of embodiments 69 to 70, wherein the antigenic unit comprises n−1 antigenic subunits, each subunit comprising a neoepitope and a subunit linker, and a terminal neoepitope, and wherein n is the number of neoepitopes in said antigenic unit and n is an integer of from 1 to 50.

72. The construct according to any of embodiments 69 to 71, wherein the neoepitopes have a length of from 7 to 30 amino acids such as from 7 to 10 amino acids (such as 7, 8, 9 or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

73. The construct according to any of embodiments 67 to 72, wherein the antigenic unit further comprises one or more patient-present shared cancer antigens or parts thereof.

74. The construct according to embodiment 73, wherein the antigenic unit further comprises one or more parts of one or more patient-present shared cancer antigens.

75. The construct according to embodiment 74, wherein said parts are epitopes.

76. The construct according to any of embodiments 74 to 75, wherein the antigenic unit further comprises several patient-present shared cancer epitopes.

77. The construct according to any of embodiments 73 to 76, wherein the patient-present shared cancer antigen is selected or the patient-present shared cancer epitope is a part of a patient-present shared cancer antigen selected from the group consisting of overexpressed cellular proteins, aberrantly expressed cellular proteins, cancer testis antigens, differentiation antigens, viral antigens, mutated oncogenes, mutated tumor suppressor genes, oncofetal antigens, shared fusion antigens, shared intron retention antigens, shared antigens caused by frameshift mutation, dark matter antigens and shared antigens caused by spliceosome mutations.

78. The construct according to any of embodiments 1 to 66, wherein the antigenic unit comprises one or more patient-present shared cancer antigens or parts thereof.

79. The construct according to embodiment 78, wherein the antigenic unit comprises one or more parts of one or more patient-present shared cancer antigens.

80. The construct according to embodiment 79, wherein said parts are epitopes.

81. The construct according to embodiment 80, wherein the antigenic unit comprises several epitopes.

82. The construct according to any of embodiments 78 to 81, wherein the patient-present shared cancer antigen is selected or the patient-present shared cancer epitope is a part of a patient-present shared cancer antigen selected from the group consisting of overexpressed cellular proteins, aberrantly expressed cellular proteins, cancer testis antigens, differentiation antigens, viral antigens, mutated oncogenes, mutated tumor suppressor genes, oncofetal antigens, shared fusion antigens, shared intron retention antigens, shared antigens caused by frameshift mutation, dark matter antigens and shared antigens caused by spliceosome mutations.

83. The construct according to any of embodiments 1 to 66, wherein the antigenic unit comprises one or more shared cancer antigens or parts thereof.

84. The construct according to embodiment 83, wherein the antigenic unit comprises one or more parts of one or more shared cancer antigens.

85. The construct according to embodiment 84, wherein said parts are epitopes.

86. The construct according to embodiment 85, wherein the antigenic unit comprises several epitopes.

87. The construct according to any of embodiments 83 to 86, wherein the shared cancer antigen is selected or the shared cancer epitope is part of a shared cancer antigen selected from the group consisting of overexpressed cellular proteins, aberrantly expressed cellular proteins, cancer testis antigens, differentiation antigens, viral antigens, mutated oncogenes, mutated tumor suppressor genes, oncofetal antigens, shared fusion antigens, shared intron retention antigens, shared antigens caused by frameshift mutation, dark matter antigens and shared antigens caused by spliceosome mutations.

88. The construct according to any of embodiments 1 to 66, wherein the antigenic unit comprises one or more infectious antigens or parts thereof.

89. The construct according to embodiment 88, wherein the antigenic unit comprises one or more full-length infectious antigens or one or more parts of such full-lengths infectious antigens or one or more full-length infectious antigens and one or more parts of full-lengths infectious antigens.

90. The construct according to any of embodiments 88 to 89, wherein the antigenic unit comprises one or more parts of one or more full-length infectious antigens.

91. The construct according to embodiment 90, wherein such parts are B cell epitopes and the antigenic unit comprises one or more B cell epitopes from one or more infectious antigens.

92. The construct according to embodiment 90, wherein such parts are T cell epitopes and the antigenic unit comprises one or more T cell epitopes from one or more infectious antigens.

93. The construct according to embodiment 90, wherein the antigenic unit comprises one or more B cell epitopes and one or more T cell epitopes of one infectious agent.

94. The construct according to embodiment 90, wherein the antigenic unit comprises one or more B cell epitopes and one or more T cell epitopes of several different infectious agents.

95. The construct according to embodiment 88 to 92 or 94 to 95, wherein the antigenic unit comprises (i) one or more full-length infectious antigens or one or more parts of such antigens and (ii) one or more T cell epitopes from one or more infectious antigens.

96. The construct according to embodiments 88 to 92 or 94 to 95, wherein the construct comprises several infectious antigens which are from the same pathogen.

97. The construct according to embodiment 88 to 92 or 94 to 95, wherein the construct comprises several infectious agents which are from different pathogens.

98. The construct according to any of embodiments 1 to 97, wherein the construct comprises a unit linker.

99. The construct according to embodiment 98, wherein the unit linker is a non-immunogenic linker and/or flexible or rigid linker.

100. The construct according to any of the previous embodiments, wherein the nucleotide sequence further encodes a signal peptide or wherein the polypeptide further comprises a signal peptide.

101. The construct according to embodiment 100, wherein the signal peptide is the natural leader sequence of the hCCL3 or hCCL3L1.

102. The construct according to any of embodiments 100 to 101, wherein the targeting unit is mutated hCCL3 and the signal peptide comprises an amino acid sequence having at least 85% sequence identity to the amino acid sequence of SEQ ID NO: 116 or wherein the targeting unit is mutated hCCL3L1 and the signal peptide comprises an amino acid sequence having at least 85% sequence identity to the amino acid sequence of SEQ ID NO: 117.

103. The construct according to embodiment 102, wherein the targeting unit is mutated hCCL3 and the signal peptide consists of an amino acid sequence having at least 85% sequence identity to the amino acid sequence of SEQ ID NO: 116 or wherein the targeting unit is mutated hCCL3L1 and the signal peptide consists of an amino acid sequence having at least 85% sequence identity to the amino acid sequence of SEQ ID NO: 117

104. The construct according to embodiment 103, the targeting unit is mutated hCCL3 and the signal peptide has the amino acid sequence of SEQ ID NO: 116 or wherein the targeting unit is mutated hCCL3L1 and the signal peptide has the amino acid sequence of SEQ ID NO: 117.

105. The construct according to any of embodiments 100 to 104, wherein the targeting unit has an amino acid sequence as defined in any of embodiments 42 to 45 and the signal peptide comprises an amino acid sequence having at least 85% sequence identity to the amino acid sequence of SEQ ID NO: 117 or wherein the targeting unit has an amino acid sequence as defined in any of embodiments 47 to 50 and the signal peptide comprises an amino acid sequence having at least 85% sequence identity to the amino acid sequence of SEQ ID NO: 116.

106. The construct according to embodiment 105, wherein the targeting unit has an amino acid sequence as defined in any of embodiments 42 to 45 and the signal peptide consists of an amino acid sequence having at least 85% sequence identity to the amino acid sequence of SEQ ID NO: 117 or wherein the targeting unit has an amino acid sequence as defined in any of embodiments 47 to 50 and the signal peptide consists of an amino acid sequence having at least 85% sequence identity to the amino acid sequence of SEQ ID NO: 116

107. The construct according to embodiment 106, wherein the targeting unit has an amino acid sequence as defined in any of embodiments 42 to 45 and the signal peptide comprises the amino acid sequence of SEQ ID NO: 117 or wherein the targeting unit has an amino acid sequence as defined in any of embodiments 47 to 50 and the signal peptide comprises the amino acid sequence of SEQ ID NO: 116.

108. The construct according to embodiment 107, wherein the targeting unit has an amino acid sequence as defined in any of embodiments 42 to 45 and the signal peptide has the amino acid sequence of SEQ ID NO: 117 or wherein the targeting unit has an amino acid sequence as defined in any of embodiments 47 to 50 and the signal peptide has the amino acid sequence of SEQ ID NO: 116.

109. The construct according to any of embodiments 1 to 108, wherein the construct is the polynucleotide or wherein the construct is the polypeptide.

110. A polynucleotide as defined in any of embodiments 1 to 109.

111. The polynucleotide according to embodiment 110, wherein the polynucleotide is a DNA polynucleotide, such as genomic DNA or cDNA, either double-stranded or single-stranded, or synthetic DNA, such as DNA amplicons, such as closed DNA strands, such as doggybone DNA and linear DNA amplicons.

112. The polynucleotide according to embodiment 110, wherein the polynucleotide is a RNA polynucleotide, such as mRNA.

113. A vector comprising the polynucleotide according to any of embodiments 110 to 112.

114. The vector according to embodiment 113, wherein the vector is a polycistronic vector which comprises: a) the polynucleotide according to embodiment 110 and b) one or more nucleic acid sequences encoding one or more immunostimulatory compounds, wherein the vector allows for the co-expression of a polypeptide encoded by the polynucleotide and the one or more immunostimulatory compounds as separate molecules.

115. The vector according to embodiment 114, wherein vector comprises one or more co-expression elements, such as co-expression elements selected from the group consisting of IRES elements, 2A peptides, promoters and bidirectional promoters.

116. The vector according to any of embodiments 114 or 115, wherein the one or more immunostimulatory compound is a compound that stimulates antigen presenting cells (APCs) and the stimulation results in attraction, activation, maturation and/or proliferation of APCs.

117. The vector according to any of embodiments 113 to 116, wherein the vector is selected from the group consisting of DNA vector and RNA vector.

118. The vector according to embodiment 117, wherein the vector is a DNA vector, such as a DNA plasmid or DNA viral vector, such as a DNA viral vector selected from the group consisting of adenovirus, vaccinia virus, adeno-associated virus, cytomegalovirus and Sendai virus.

119. The vector according to embodiment 117, wherein the vector is an RNA vector, such as an RNA plasmid or RNA viral vector, such as a retroviral vector, such as a retroviral vector selected from the group consisting of alphavirus, lentivirus, Moloney murine leukemia virus and rhabdovirus.

120. A method of producing the vector as defined in any of embodiments 113 to 119, the method comprising:
a) transfecting cells in vitro with the vector;
b) culturing said cells;
c) optionally, lysing the cells to release the vector from the cells; and
d) collecting and optionally purifying the vector.

121. A host cell comprising the polynucleotide according to embodiment 110 or the vector according to any of embodiments 113 to 119.

122. A polypeptide as defined in any of embodiments 1 to 109.

123. A multimeric protein consisting of multiple polypeptides as defined in any of embodiments 3 to 109.

124. The multimeric protein according to embodiment 123, which is a dimeric protein consisting of two polypeptides as defined in any of embodiments 3 to 109, such as a homodimeric protein or heterodimeric protein, preferably a homodimeric protein.

125. A method for preparing the polypeptide according to embodiment 122, the method comprises:
a) transfecting or transducing cells with the polynucleotide according to any of embodiments 110 to 112 or the vector according to any of embodiments 113 to 119;
b) culturing the cells;
c) isolating the polypeptide from the cells; and
d) optionally purifying the isolated polypeptide.

126. A method for preparing the multimeric protein according to any of embodiments 123 to 124, the method comprises:
a) transfecting or transducing cells with the polynucleotide according to any of embodiments 110 to 112 or the vector according to any of embodiments 113 to 119;
b) culturing the cells;
c) isolating the multimeric protein from the cells; and
d) optionally purifying the isolated multimeric protein.

127. The construct according to any of embodiments 1 to 109, the polynucleotide according to any of embodiments 110 to 112, the vector according to any of embodiments 113 to 119, the polypeptide according to embodiments 122 or the multimeric protein according to any of embodiments 123 to 124 for use as a medicament.

128. A pharmaceutical composition comprising i) a pharmaceutically acceptable carrier and ii) the construct according to any of embodiments 1 to 109, the polynucleotide according to any of embodiments 110 to 112, the vector according to any of embodiments 113 to 119, the polypeptide according to embodiments 122 or the multimeric protein according to any of embodiments 123 to 124.

129. The pharmaceutical composition according to embodiment 128, comprising i) a pharmaceutically acceptable carrier and ii) the polynucleotide according to any of embodiments 110 to 112, the vector according to any of embodiments 113 to 119, the polypeptide according to embodiments 122 or the multimeric protein according to any of embodiments 123 to 124.

130. The pharmaceutical composition according to embodiment 130, comprising a pharmaceutically acceptable carrier and the vector according to any of embodiments 113 to 119.

131. The pharmaceutical composition according to any of embodiments 128 to 130, wherein the pharmaceutically acceptable carrier is selected from the group consisting of saline, buffered saline, such as PBS, dextrose, water, glycerol, ethanol, aqueous buffers, such as isotonic aqueous buffers or Tyrode's buffer, and combinations thereof.

132. The pharmaceutical composition according to any of embodiments 128 to 131, wherein the composition further comprises molecules that facilitate the transfection of cells with the polynucleotide or vector.

133. The pharmaceutical composition according to any of embodiments 128 to 132, wherein the composition further comprises a pharmaceutically acceptable amphiphilic block co-polymer comprising blocks of poly(ethylene oxide) and polypropylene oxide, such as comprising said pharmaceutically acceptable amphiphilic block co-polymer in an amount of from 0.2% w/v to 20% w/v.

134. The pharmaceutical composition according to any of embodiments 128 to 129, 131 and 133, wherein the composition comprises the polypeptide according to embodiment 122 or the multimeric protein according to any of embodiments 123 to 125, preferably in a range of from 5 μg to 5 mg.

135. The pharmaceutical composition according to any of embodiments 128 to 133, wherein the composition comprises the polynucleotide according to any of embodiments 110 to 112 or the vector according to any of embodiments 113 to 119 in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg.

136. A method of treating a subject having a disease or being in need of prevention of a disease, the method comprising administering to the subject the construct according to any of embodiments 1 to 109 or the polynucleotide according to any of embodiments 110 to 112 or the vector according to any of embodiments 113 to 119 or the polypeptide according to embodiments 122 or the multimeric protein according to any of embodiments 123 to 124 or the pharmaceutical composition according to any of embodiments 128 to 135, wherein the antigenic unit comprised in or encoded by said construct, polynucleotide, vector, polypeptide or multimeric protein comprises one or more antigens or parts thereof, which are relevant for said disease.

137. The method according to embodiment 136, wherein the method comprises administering to the subject the pharmaceutical composition according to any of embodiments 128 to 135.

138. The method according to embodiment 137, wherein the pharmaceutical composition is administered in a therapeutically or prophylactically effective amount.

139. The method according to any of embodiments 137 to 138, wherein the pharmaceutical composition is administered by intradermal, intramuscular, or subcutaneous injection, or by mucosal or epithelial application, such as intranasal or oral.

140. A method of treating a subject having cancer, the method comprising administering to the subject the pharmaceutical composition as defined in any of embodiments any of embodiments 128 to 135, wherein the composition comprises the polynucleotide or polypeptide as defined in any of embodiments 1 to 87 and 98 to 109 or a vector comprising such polynucleotide or the multimeric protein consisting of multiple such polypeptides.

141. The method according to embodiment 140, wherein the pharmaceutical composition is administered in a therapeutically effective amount.

142. The method according to any of embodiments 140 to 141, wherein the pharmaceutical composition is administered by intradermal, intramuscular, or subcutaneous injection, or by mucosal or epithelial application, such as intranasal or oral.

143. The method according to any of embodiments 140 to 142, wherein the cancer is a liquid or solid cancer, such as a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

144. A method for treating a subject having an infectious disease or being in need of prevention of an infectious disease, the method comprising administering to the subject the pharmaceutical composition as defined in any of embodiments 128 to 135, wherein the composition comprises the polynucleotide or polypeptide as defined in any of embodiments 1 to 66 and 88 to 109 or a vector comprising such polynucleotide or a multimeric protein consisting of multiple of such polypeptides.

145. The method according to embodiment 144, wherein the pharmaceutical composition is administered in a therapeutically or prophylactically effective amount.

146. The method according to any of embodiments 144 to 145, wherein the pharmaceutical composition is administered by intradermal, intramuscular, or subcutaneous injection, or by mucosal or epithelial application, such as intranasal or oral.

1. A construct, the construct being:
(i) a polynucleotide comprising a nucleotide sequence encoding a targeting unit selected from the group consisting of mutated human CCL3 (hCCL3) and mutated human CCL3L1 (hCCL3L1) and an antigenic unit comprising one or more antigens or parts thereof; or
(ii) a polypeptide encoded by the nucleotide sequence as defined in (i),
wherein the amino acid sequence of the targeting unit comprises one or more mutations, compared to the amino acid sequence of the wild type, which reduce or prevent the formation of targeting unit oligomers.

2. The construct as claimed in claim 1, the construct being:
(i) the polynucleotide comprising a nucleotide sequence encoding a targeting unit selected from the group consisting of mutated human CCL3 (hCCL3) and mutated human CCL3L1 (hCCL3L1), a multimerization unit, such as a dimerization unit and an antigenic unit comprising one or more antigens or parts thereof; or
(ii) a polypeptide encoded by the nucleotide sequence as defined in (i); or
(iii) a multimeric protein consisting of multiple polypeptides as defined in (ii), such as a dimeric protein consisting of two polypeptides,
wherein the amino acid sequence of the targeting unit comprises one or more mutations, compared to the amino acid sequence of the wild type, which reduce or prevent the formation of targeting unit oligomers.

3. The construct as claimed in any of the preceding claims, wherein the one or more mutations reduce or prevent the formation of targeting unit oligomers in solution.

4. The construct as claimed in any of the preceding claims, wherein the mutation is a substitution of an amino acid comprised in the wild type amino acid sequence with a different amino acid.

5. The construct as claimed in any of the preceding claims, wherein the mutation does not lead to a decreased solubility and/or decreased ability of receptor binding and/or decreased ability of receptor activation of the mutated hCCL3 or hCCL3L1 targeting unit, compared to the solubility and/or ability of receptor binding and/or ability of receptor activation of the hCCL3 or hCCL3L1 wild type targeting unit.

6. The construct as claimed in any of the preceding claims, wherein the mutation is designed to:
a) disrupt the salt bridge formed by amino acid residues D27 and R46 or the salt bridge formed by amino acid residues E67 and R48 in crystalized polymers formed by the interaction of hCCL3 wild type monomers or hCCL3L1 wild type monomers or;
b) disrupt the hydrogen bond between amino acid residues D6 and S33 in crystalized polymers formed by the interaction of hCCL3 wild type monomers or hCCL3L1 wild type monomers; or
c) disrupt the hydrophobic interactions of amino acid residue F24 or F29 or Y28 in crystalized polymers formed by the interaction of hCCL3 wild type monomers or hCCL3L1 wild type monomers.

7. The construct as claimed in any of the preceding claims, wherein one or more of the amino acids D27, E67, D6, F24, F29 and Y28 of the amino acid sequence of the wild type hCCL3 or hCCL3L1 is substituted with a small, non-polar amino acid, such as an amino acid selected from the group consisting of G, A, V, L and I.

8. The construct as claimed any of the preceding claims, wherein one or more of the amino acids D27, E67, D6, F24, F29 and Y28 of the amino acid sequence of the wild type hCCL3 or hCCL3L1 is substituted with alanine.

9. The construct as claimed in any of the preceding claims, wherein the amino acid P8 of amino acid sequence of the wild type hCCL3 or hCCL3L1 is substituted with a non-polar amino acid, such as a small, non-polar amino acid.

10. The construct as claimed in any of the preceding claims, wherein the amino acid P8 of the amino acid sequence of wild type hCCL3 or hCCL3L1 is substituted with alanine.

11. The construct as claimed in any of the preceding claims, wherein the targeting unit comprises one or more mutations selected from the group consisting of D27A, E67A, D6A, F24A, F29A, Y28A and P8A.

12. The construct as claimed in any of the preceding claims, wherein the targeting unit comprises a D27A mutation and an E67A mutation.

13. The construct as claimed in any of the preceding claims, wherein the targeting unit comprises a D27A mutation, an E67A mutation and a P8A mutation.

14. The construct as claimed in any of the preceding claims, wherein the targeting unit is mutated hCCL3L1.

15. The construct as claimed in any of claims 1 to 13, wherein the targeting unit is mutated hCCL3.

16. The construct as claimed in claim 14, wherein the targeting unit comprises an amino acid sequence selected from the group consisting of SEQ ID NO: 5, SEQ ID NO: 6, SEQ ID NO: 8, SEQ ID NO: 141, amino acid 3-70 of SEQ ID NO: 5, amino acid 3-70 of SEQ ID NO: 6, amino acid 3-70 of SEQ ID NO: 8 and amino acid 3-70 of SEQ ID NO: 141.

17. The construct as claimed in claim 14, wherein the targeting unit consists of the amino acid sequence selected from the group consisting of SEQ ID NO: 5, SEQ ID NO: 6, SEQ ID NO: 8, SEQ ID NO: 141, amino acid 3-70 of SEQ ID NO: 5, amino acid 3-70 of SEQ ID NO: 6, amino acid 3-70 of SEQ ID NO: 8 and amino acid 3-70 of SEQ ID NO: 141.

18. The construct as claimed in claim 14, wherein the construct is the polynucleotide comprising a nucleotide sequence selected from the group of SEQ ID NO: 139, SEQ ID NO: 188, SEQ ID NO: 189 and SEQ ID NO: 190, which encodes the targeting unit.

19. The construct as claimed in claim 15, wherein the targeting unit comprises an amino acid sequence selected from the group consisting of SEQ ID NO: 3, SEQ ID NO: 4, SEQ ID NO: 7 and SEQ ID NO: 140.

20. The construct as claimed in claim 15, wherein the targeting unit consists of the amino acid sequence selected from the group consisting of SEQ ID NO: 3, SEQ ID NO: 4, SEQ ID NO: 7 and SEQ ID NO: 140.

21. The construct according to any of claims 2 to 20, wherein the multimerization unit is selected from the group consisting of dimerization unit, trimerization unit and tetramerization unit and wherein said multimerization unit optionally comprises a hinge region which has the ability to form one or more covalent bonds.

22. The construct as claimed in any of any of claims 2 to 21, wherein the multimerization unit comprises a hinge region which has the ability to form one or more covalent bonds, preferably an Ig derived hinge region, such as derived from human Ig, such as derived from hIgG1 or hIgG2 or hIgG3 or from hIgM.

23. The construct according to any of claims 2 to 22, wherein the multimerization unit is a dimerization unit and said dimerization unit further comprises another domain that facilitates dimerization.

24. The construct according to claim 23, wherein the other domain is an immunoglobulin domain, preferably an immunoglobulin constant domain, more preferably a carboxyterminal C domain derived from IgG, preferably from IgG3, more preferably from hIgG3.

25. The construct according to any of claims 23 to 24, wherein the dimerization unit further comprises a dimerization unit linker, such as glycine-serine rich linker, such as GGGSSGGGSG, preferably wherein the dimerization unit linker connects the hinge region and the other domain that facilitates dimerization.

26. The construct according to any of claims 23 to 25, wherein the dimerization unit comprises hinge exon h1 and hinge exon h4, a dimerization unit linker and a CH3 domain of human IgG3.

27. The construct according to any of the preceding claims, wherein the antigenic unit comprises one or more disease-relevant antigens or parts thereof.

28. The construct according to any of the preceding claims, wherein the antigenic unit comprises one or more neoantigens or parts thereof, preferably one or more neoepitopes.

29. The construct according to claim 28, wherein the antigenic unit further comprises one or more patient-present shared cancer antigens or parts thereof, preferably one or more patient-present shared cancer epitopes.

30. The construct according to any of claims 1 to 27, wherein the antigenic unit comprises one or more shared cancer antigens or parts thereof, preferably one or more shared cancer epitopes.

31. The construct according to any of claims 1 to 27, wherein the antigenic unit comprises one or more infectious antigens or parts thereof.

32. The construct according to claim 31, wherein the antigenic unit comprises one or more full-length infectious antigens or one or more parts of such full-lengths infectious antigens or one or more full-length infectious antigens and one or more parts of full-lengths infectious antigens.

33. The construct according to claim 32, wherein such parts are B cell epitopes and the antigenic unit comprises one or more B cell epitopes from one or more infectious antigens and/or wherein such parts are T cell epitopes and the antigenic unit comprises one or more T cell epitopes from one or more infectious antigens.

34. The construct according to any of the preceding claims, wherein the construct comprises a unit linker, preferably a non-immunogenic unit linker and/or flexible or rigid unit linker.

35. The construct according to any of the preceding claims, wherein the nucleotide sequence further encodes a signal peptide.

36. The construct according to claim 35, wherein the signal peptide is the natural leader sequence of hCCL3 or hCCL3L1.

37. A polynucleotide as defined in any of claims 1 to 36, such as a DNA polynucleotide or a RNA polynucleotide.

38. A vector comprising the polynucleotide according to claim 37.

39. The vector according to claim 38, wherein the vector is a polycistronic vector which comprises: a) the polynucleotide according to claim 37 and b) one or more nucleic acid sequences encoding one or more immunostimulatory compounds, wherein the vector allows for the co-expression of a polypeptide encoded by the polynucleotide and the one or more immunostimulatory compounds as separate molecules.

40. A method of producing the vector according to any of claims 38 to 39, the method comprising:
a) transfecting cells in vitro with the vector;
b) culturing said cells;
c) optionally, lysing the cells to release the vector from the cells; and
d) collecting and optionally purifying the vector.

41. A host cell comprising the polynucleotide according to claim 37 or the vector according to any of claims 38 to 39.

42. A polypeptide encoded by the polynucleotide according to claim 37.

43. A multimeric protein as defined in any of claims 2 to 36.

44. A method for preparing the polypeptide according to claim 42, the method comprises:
a) transfecting or transducing cells with the polynucleotide according to claim 37 or the vector according to any of claims 38 to 39;
b) culturing the cells;
c) isolating the polypeptide from the cells; and
d) optionally purifying the isolated polypeptide.

45. A method for preparing the multimeric protein according to claim 43, the method comprises:
a) transfecting or transducing cells with the polynucleotide according to claim 37 or the vector according to any of claims 38 to 39;
b) culturing the cells;
c) isolating the multimeric protein from the cells; and
d) optionally purifying the isolated multimeric protein.

46. The construct according to any of claims 1 to 36, the polynucleotide according to claim 37, the vector according to any of claims 38 to 39, the polypeptide according to claim 42 or the multimeric protein according to claim 43 for use as a medicament.

47. A pharmaceutical composition comprising i) a pharmaceutically acceptable carrier and ii) the construct according to any of claims 1 to 36, the polynucleotide according to claim 37, the vector according to any of claims 38 to 39, the polypeptide according to claim 42 or the multimeric protein according to claim 43

48. A method of treating a subject having a disease or being in need of prevention of a disease, the method comprising administering to the subject the construct according to any of claims 1 to 36, the polynucleotide according to claim 37, the vector according to any of claims 38 to 39, the polypeptide according to claim 42 or the multimeric protein according to claim 43 or the pharmaceutical composition according to claim 47, wherein the antigenic unit comprised in or encoded by said construct, polynucleotide, vector, polypeptide or multimeric protein comprises one or more antigens or parts thereof, which are relevant for said disease.

49. A method of treating cancer in a subject, the method comprising administering to the subject the pharmaceutical composition according to claim 47, wherein the composition comprises the polynucleotide or polypeptide or multimeric protein as defined in any of claims 1 to 30, or a vector comprising such polynucleotide.

50. A method for treating a subject having an infectious disease or being in need of prevention of an infectious disease, the method comprising administering to the subject the pharmaceutical composition according to claim 47, wherein the composition comprises the polynucleotide or polypeptide or multimeric protein as defined in any of claims 1 to 29 or 32 to 33, or a vector comprising such polynucleotide
